(19) United States
(12) Patent Application Publication
(10) Pub. No.: US 2024/0033336 A1
(43) Pub. Date: Feb. 1, 2024

(54) TUMOR ANTIGENS, COMPOUNDS COMPRISING THE TUMOR ANTIGENS AND USES THEREOF

(71) Applicant: Boehringer Ingelheim International GmbH, Ingelheim Am Rhein (DE)

(72) Inventors: Elodie BELNOUE, Geneva (CH); Madiha DEROUAZI, Grand-Saconnex (CH); Paul ADAM, Ingelheim Am Rhein (DE); Imgard Rita Maria HOFMANN, Ingelheim Am Rhein (DE); Ralf LEONHARDT, Ingelheim Am Rhein (DE); Samuel LUKOWSKI, Ingelheim Am Rhein (DE); Tobias NOLDEN, Ingelheim Am Rhein (DE); Francesca TRAPANI, Ingelheim Am Rhein (DE)

(21) Appl. No.: 18/184,000

(22) Filed: Mar. 15, 2023

(30) Foreign Application Priority Data
Mar. 16, 2022 (EP) 22162533.8
Oct. 31, 2022 (EP) 22204783.9

(51) Int. Cl. A61K 39/00; A61K 39/395; A61P 35/00

(52) U.S. Cl. CPC A61K 39/001164; A61K 39/001162; A61K 39/0011; A61K 39/4611; A61K 39/4632; A61K 39/4631; A61K 39/001182; A61K 39/001154; A61K 39/464464; A61K 39/464462; A61K 39/4644; A61K 39/3955; A61P 35/00; A61K 2039/5154; A61K 2039/53; A61K 2039/70; A61K 2039/5256; A61K 2039/54

(57) ABSTRACT

The present invention relates to tumor antigens encoded in a 5′-upstream open reading frame (uORF) within the 5′ UTR of different mRNAs. Compositions and peptides comprising such tumor antigens and a virus encoding such tumor antigens are provided. The present invention also relates to the use of such compositions, peptides and viruses in the treatment of cancer.

### PRIORITY STATEMENT

This patent application claims priority under 35 USC § 119 to European Application No.: 22162533.8 filed 16 Mar. 2022 and European Application No.: 22204783.9 filed 31 Oct. 2022. The contents of each application recited above are incorporated herein by reference in their entirety.

### FIELD

The present invention relates to the field of vaccination and immunotherapy, in particular to cancer immunotherapy. More specifically, the present invention relates to tumor antigens encoded in a 5′-upstream open reading frame (uORF) within the 5′ UTR of different mRNAs. Compositions and peptides comprising such tumor antigens and a virus encoding such tumor antigens are provided.

The present invention also relates to the use of such compositions, peptides and viruses in the treatment of cancer.

### REFERENCE TO AN ELECTRONIC SEQUENCE LISTING

The contents of the electronic sequence listing (ST26_SL_9_Mar_2023.xml; Size: 90 KB; and Date of Creation: 9 Mar. 2023) is herein incorporated by reference in its entirety.

### BACKGROUND

The immune system can recognize and to some extent eliminate tumor cells, however, this anti-tumor response is often of low amplitude and inefficient. Boosting this weak anti-tumor response with therapeutic vaccination has been a long sought goal for cancer therapy. Modulating the immune system to enhance immune responses has thus become a promising therapeutic approach in oncology as it can be combined with standard of care treatments.

Tumor cells commonly express several antigens, such as tumor-associated antigens (TAAs), viral antigens (oncovirus) or mutation-derived antigens (neoantigens). Various TAAs, viral antigens or neoantigens expressed in cancer cells have been identified and utilized as targets for cancer vaccines. One approach to elicit tumor-specific immune responses is the peptide-based cancer vaccination involving administration of TAAs, viral antigens or neoantigen-derived to treat cancer according to the nature of the tumor.

While tumor associated antigens are usually also present on normal cells, tumor-specific antigens are present only on tumor cells. Tumor-specific antigens include mutation-derived antigens as well as antigens derived from cryptic open reading frames and/or frameshift mutations. Such antigens that can be recognized by the immune system as foreign antigens and, therefore, drive an anti-tumor immune response in animal tumor-models and cancer patients. However, neoantigens with tumor-specific mutations often require vaccines tailored to individual patients, which is cumbersome and cost-intensive. Recently, so-called “dark antigens” received increasing interest, which are peptides expressed specifically in tumors, which are encoded in sequence regions previously believed to be “non-coding”, such as 5′ UTRs of mRNAs.

### SUMMARY

In view thereof, it is the object of the present invention to provide novel tumor antigens, which are specifically expressed in tumors of many patients. Such tumor antigens provide tumor specificity, but do not require cumbersome and cost-intensive personalized approaches. It is also an object of the present invention to provide vaccines, in particular vaccines which can be used in a heterologous prime-boost regimen, combining such tumor antigens with further advantageous features, such as a multi-antigenic domain. This is especially relevant as each human being has a different set of MHC molecules and presentation of multiple peptides is allowing representation of the best (highest affinity) peptides in the respective HLA.

This object is achieved by means of the subject-matter set out below and in the appended claims.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1A-1C shows for Example 1 KRAS-uORF1-, TPX2-uORF1- and AURKA-uORF2-encoding transcript expression as measured by BaseScope on tissue arrays (HalioDX) and tumor whole sections (FIG. 1A). Probes corresponding to the KRAS-uORF1-, TPX2-uORF1- and AURKA-uORF2-antigen sequences were employed. Bar diagrams display the quantification of the experiments through an automated scoring algorithm (HALO-Indica lab). Note that CRC-adjacent healthy tissue is characterized by substantially higher expression of the transcripts of interest than normal healthy colon tissue. (FIG. 1B) RNA-Seq-based expression of KRAS, TPX2, and AURKA transcripts in healthy normal colon/pancreas versus tumor-adjacent colon/pancreas tissue (TCGA/GTEx). mRNA expression of KRAS, TPX2 and AURKA data were extracted from the following public databases: the Cancer Genome Atlas (TCGA) and the Genotype-Tissue Expression (GTEx). (FIG. 1C) Translation of KRAS-uORF1-, TPX2-uORF1- and AURKA-uORF2-tumor antigen ORFs in human pancreatic (PDAC), gastric (GC), and colorectal (CRC) tumors or tumor-adjacent healthy tissue. Translation was measured by RiboSeq. The number of in-frame reads across the respective ORFs was normalized for ORF length (assuming translation is driven by the innermost possible start codon) and sample library size (i.e., the sum of all trimmed reads in the sample mapping to the human genome after removal of rRNA- and tRNA-derived reads). An unpaired two-tailed t-test was performed to assess statistical significance (*p<0.05; **p<0.01; ***p<0.001; ****p<0.0001).

FIG. 2A-2C shows for Example 2 (FIG. 2A) Expression of DUOXA2 and CEACAM5 transcripts in various GI tract tumors as well as in health tissue (TCGA/GTEx). (FIG. 2B) CEACAM5 protein expression as measured by immunohistochemistry staining on tissue arrays (HalioDX). The bar diagram (upper panel) displays the quantification of the experiment. (FIG. 2C) DUOXA2 transcript expression as measured by RNAScope on tissue arrays (HalioDX). The bar diagram (left panel) displays the quantification of the experiment.

FIG. 3A-3B shows for Example 3 (FIG. 3A) Co-expression on the mRNA level of mutant KRAS-G12D/V with CEACAM5 and DUOXA2 in pancreatic (PDAC) and colorectal (CRC) cancer (based on various RNASeq-based datasets including TCGA). (FIG. 3B) Immunogenicity of epitopes derived from KRAS-uORF1, TPX2-uORF1, AURKA-uORF2, KRAS-G12D/V as well as for the DUOXA2. In vitro priming was performed employing the indicated peptides (e.g., KLVVVGADGV) in the context of the given HLA alleles (e.g., HLA-A*0201) to stimulate specific T cell responses among healthy donor PBMCs. Cultures thereby enriched for peptide-specific T cells were subsequently assayed on various tumor cell line targets pulsed with the respective peptides (specific response). In parallel, the same tumor cell lines were pulsed either with an irrelevant control peptide or were not pulsed at all (unspecific background response). T cell activation was measured by IFNγ ELISpot. In panel (B) each spot corresponds to a different tumor cell line or moDC target. Displayed is the ratio of the specific response to the unspecific background response. A ratio above 1 indicates a specific T cell response. A negative control (i.e., a peptide epitope that did not elicit a specific T cell response) is included in the panel.

FIG. 4 shows for Example 4 the presentation of ATP150- and ATP152-derived epitopes by human moDCs. moDCs were incubated with either ATP150 or ATP152 to allow processing of the vaccine by the endogenous antigen presentation machinery. Cross-presented antigen was subsequently analyzed by mass spectrometry. In the panel, detected peptide epitopes were mapped onto the ATP150/ATP152 backbone to visualize the region from which they are derived. Each line corresponds to one of the 10 PBMCs' donors. Note that the 10 donors differ from each other by their HLA haplotype and are, hence, expected to present different peptide epitopes.

FIG. 5A-5B shows for Example 5 the immunogenicity after ATP150/VSV-GP154 heterologous prime-boost vaccination. IFNγ-producing CEACAM5- and DUOXA2-specific T cells by ELISpot (FIG. 5A) Outline of the prime-boost protocol. Mice [n=5] were either both primed (d0) and boosted (d14) with 10 nmol ATP150 (s.c.) or primed (d0) with 10 nmol ATP150 (s.c.) and boosted (d14) with 10⁷ TCID₅₀ VSV-GP154 (i.v.). (FIG. 5B) On day 21, splenocytes were harvested and pulsed with overlapping 11mer peptides corresponding to the CEACAM5 and DUOXA2 regions incorporated in the vaccine. ***p<0.001; ****p<0.0001.

FIG. 6A-6B shows for Example 6 (FIG. 6A) Tumor growth (mean±SEM) and (FIG. 6B) survival of mice injected with 1×10⁵ TC-1 cells s.c. and immunized with 2 nmol the peptide construct (multi-antigenic domain “Mad25”) s.c. 7 days later or with 1×10⁷ TCID₅₀ VSV-GP-HPV i.v. 14 days post tumor implantation. Additional doses of K and V were administered as indicated by dotted lines and tumor growth was monitored (n=7).

FIG. 7A-7B shows for Example 7 the vaccination schedule (FIG. 7A) and detection of HPV-specific CD8 T cells (FIG. 7B) after heterologous prime-boost and anti-PD-1 in TC-1 tumor-bearing mice. Mice were bled at days 14, 21, 35, 42, 56, and multimer staining detecting HPV-specific CD8 T cells was performed and analyzed by flow cytometry.

FIG. 8A-8B shows for Example 7 the efficacy of heterologous prime-boost vaccination and anti-PD-1 in TC-1 tumor-bearing mice. Mean tumor volume (FIG. 8A) and survival curve (FIG. 8B) of C57BL/6 mice (n=7 mice/group) implanted s.c. with TC-1 cells. Mice were vaccinated with the peptide construct (multi-antigenic domain “Mad25”) (K, 2 nmol/mouse; s.c.) and VSV-GP-HPV (V; 10⁷ TCID₅₀/mouse; i.v.). Anti-PD-1 (200 ug) was administrated i.v. Numbers of tumor-free mice are indicated.

FIG. 9 shows for Example 8 the efficacy of heterologous prime-boost vaccination comparing three different doses of VSV-GP-HPV in tumor-bearing mice. Tumor volumes (upper/left panels) and survival curves (lower/right panel) of of C57BL/6 mice (n=7 mice/group) implanted s.c. with TC-1 cells are shown. Mice were vaccinated at d7, d14 and d28 (Vac) with either the peptide construct (multi-antigenic domain “Mad25”) (K) or VSV-GP-HPV (V) at 3 different doses, 1×10⁷, 3×10⁷ or 5×10⁷ TCID₅₀. Median survival is indicated on the graph (ms). *, p<0.05; **, p<0.01; ***, p<0.001.

### DETAILED DESCRIPTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

Throughout the present specification, the terms “peptide”, “polypeptide”, “protein” and variations of these terms are used interchangeably. These terms refer to peptides, oligopeptides, or proteins including fusion proteins, which comprise at least two amino acids joined to each other, preferably by a (“normal”) peptide bond. Alternatively, the amino acids may be joined to each other by a modified peptide bond, such as for example in the cases of isosteric peptides. Accordingly, as used herein, the term “peptide” refers to shorter (oligo)peptides as well as to longer (polypeptides) and to proteins, independently of their length. “Classical” peptides, which are typically composed of amino acids selected from the 20 amino acids defined by the genetic code, linked to each other by a normal peptide bond, are preferred. However, a peptide can also comprise amino acids other than the 20 amino acids defined by the genetic code in addition to these amino acids, or it can be composed of amino acids other than the 20 amino acids defined by the genetic code. In some embodiments, a peptide can be composed of amino acids modified by natural processes, such as post-translational maturation processes, or by chemical processes, which are well known to a person skilled in the art. Modifications can appear anywhere in the peptide: in the peptide skeleton, in the amino acid chain or even at the carboxy- or amino-terminal end of the peptide. Accordingly, the terms “peptide”, “polypeptide”, “protein” also include modified peptides, polypeptides and proteins. For example, a peptide modification can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination.

As used herein (i.e. throughout the present application), the term “sequence variant” refers to any alteration in a reference sequence. The term “sequence variant” includes nucleotide sequence variants and amino acid sequence variants. Preferably, a reference sequence is any of the sequences listed in the “Table of Sequences and SEQ ID Numbers” (Sequence listing), i.e. SEQ ID NO: 1 to SEQ ID NO: 51. In particular, a sequence variant shares (over the whole length of the sequence) at least 70% or at least 75%, preferably at least 80% or at least 85%, more preferably at least 90% or at least 95%, even more preferably at least 97% or at least 98%, particularly preferably at least 99% sequence identity with a reference sequence. Sequence identity may be calculated as described below. In particular, a sequence variant usually preserves the specific function of the reference sequence. In some embodiments, an amino acid sequence variant has an altered sequence in which one or more (e.g., 1, 2, 3, 4, 5, 6, 7, 8, 9, 10 or more) of the amino acids in the reference sequence is deleted or substituted, or one or more (e.g., 1, 2, 3, 4, 5, 6, 7, 8, 9, 10 or more) amino acids are inserted into or added to the sequence of the reference amino acid sequence. As a result of the alterations, the amino acid sequence variant has an amino acid sequence which is at least 70% or at least 75%, preferably at least 80% or at least 85%, more preferably at least 90% or at least 95%, even more preferably at least 97% or at least 98%, particularly preferably at least 99% identical to the reference sequence. For example, variant sequences which are at least 90% identical have no more than 10 alterations, i.e., any combination of deletions, insertions or substitutions, per 100 amino acids of the reference sequence. The same, of course, also applies similarly to nucleic acid sequences.

For (amino acid or nucleic acid) sequences without exact correspondence, a “% identity” of a first sequence may be determined with respect to a second sequence. In general, these two sequences to be compared may be aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length. Methods for comparing the identity and homology of two or more sequences are well known in the art. The percentage to which two sequences are identical can e.g., be determined using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs, e.g., BLAST or NBLAST program (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 183, 63-98; Pearson and Lipman (1988), Proc. Nati. Acad. Sci. U.S.A 85, 2444-2448.).

In general, substitutions for one or more amino acids present in the referenced amino acid sequence are preferably made conservatively. Examples of conservative substitutions include substitution of one aliphatic residue for another, such as lie, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity properties, are well known (Kyte and Doolittle, 1982, J. Mol. Biol. 157(1):105-132). Substitutions of one or more L-amino acids with one or more D-amino acids are to be considered as conservative substitutions in the context of the present invention. Exemplary amino acid substitutions are presented in Table 1 below:

TABLE 1

| Original residues | Examples of substitutions |
|---|---|
| Ala (A) | Val, Leu, Ile, Gly |
| Arg (R) | His, Lys |
| Asn (N) | Gln |
| Asp (D) | Glu |
| Cys (C) | Ser |
| Gln (Q) | Asn |
| Glu (E) | Asp |
| Gly (G) | Pro, Ala |
| His (H) | Lys, Arg |
| Ile (I) | Leu, Val, Met, Ala, Phe |
| Leu (L) | Ile, Val, Met, Ala, Phe |
| Lys (K) | Arg, His |
| Met (M) | Leu, Ile, Phe |
| Phe (F) | Leu, Val, Ile, Tyr, Trp, Met |
| Pro (P) | Ala, Gly |
| Ser (S) | Thr |
| Thr (T) | Ser |
| Trp (W) | Tyr, Phe |
| Tyr (Y) | Trp, Phe |
| Val (V) | Ile, Met, Leu, Phe, Ala |

As used herein, an “antigen” is any structural substance which serves as a target for the receptors of an adaptive immune response, in particular as a target for antibodies, T cell receptors, and/or B cell receptors. Preferably, the antigen is a peptide (including polypeptides and proteins). An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e., a single antigen has one or more epitopes. In the context of the present invention, the term “epitope” is mainly used to designate T cell epitopes, which are presented on the surface of an antigen-presenting cell, where they are bound to Major Histocompatibility Complex (MHC). For peptide antigens, T cell epitopes presented by MHC class I molecules are typically, but not exclusively, peptides between 8 and 11 amino acids in length, whereas MHC class II molecules present longer peptides, generally, but not exclusively, between 12 and 25 amino acids in length.

The terms “CD4⁺ epitope” or “CD4⁺-restricted epitope”, as used herein, designate an epitope recognized by a CD4⁺ T cell, said epitope in particular consisting of an antigen fragment lying in the groove of a MHC class II molecule. A single CD4⁺ epitope preferably consists of about 12-25 amino acids. It can also consist of, for example, about 8-25 amino acids or about 6-100 amino acids.

The terms “CD8⁺ epitope” or “CD8⁺-restricted epitope”, as used herein, designate an epitope recognized by a CD8⁺ T cell, said epitope in particular consisting of an antigen fragment lying in the groove of a MHC class I molecule. A single CD8⁺ epitope preferably consists of about 8-11 amino acids. It can also consist of, for example, about 8-15 amino acids or about 6-100 amino acids.

As used herein, a “fragment” of an antigen has usually a minimum length of 8 amino acids, i.e. the fragment comprises at least 8 consecutive amino acids of the antigen, preferably at least 10 consecutive amino acids of the antigen, more preferably at least 15 consecutive amino acids of the antigen, even more preferably at least 20 consecutive amino acids of the antigen, still more preferably at least 25 consecutive amino acids of the antigen and most preferably at least 30 consecutive amino acids of the antigen. An antigen fragment usually comprises one or more epitopes. Accordingly, the fragment of the antigen is typically immunogenic. A “sequence variant” of an antigen (or a fragment thereof) has usually an (amino acid) sequence which is at least 70% or at least 75%, preferably at least 80% or at least 85%, more preferably at least 90% or at least 95%, even more preferably at least 97% or at least 98%, particularly preferably at least 99% identical to the reference sequence. A “functional” sequence variant means in the context of an antigen/antigen fragment, that the function of the epitope(s), e.g., comprised by the antigen (fragment), is not impaired or abolished. In other words, a “functional” sequence variant of an antigen (fragment) is immunogenic, preferably it has substantially the same immunogenicity as the reference antigen (fragment). In some embodiments, the amino acid sequence of one or more epitope(s), e.g., comprised in the tumor antigen (fragment), is not mutated and, thus, identical to a (naturally occurring) reference epitope sequence.

It is understood that the skilled person usually selects the antigen, or the fragment thereof, in view of the disease to be treated. Accordingly, the antigen or antigenic epitope is usually associated with (or related to) the disease to be treated. A large number of antigens is known in the art in the context of specific diseases. For example, to treat a tumor/cancer, the skilled person selects a tumor antigen (or a fragment thereof), which is useful for the specific type of tumor/cancer. In some embodiments, the patient may be tested/screened for specific antigens (e.g., by using an isolated sample) to identify whether or not the cancer/tumor expresses the specific antigen.

As used herein, a “tumor antigen” is an antigen produced by cancer/tumor cells. Tumor antigens include tumor-associated antigens and tumor-specific antigens. Tumor-associated (also tumor-related) antigens (TAAs) are antigens, which are expressed in both, cancer/tumor cells and normal cells. Tumor-specific antigens (TSAs), in contrast, are antigens, which are expressed specifically by cancer/tumor cells, but not by normal cells. Examples of TSA include neoantigens, which were not present in the body before the cancer/tumor developed and are, thus, neoantigens are “new” to the immune system. Neoantigens are often due to somatic mutations. Suitable epitopes of tumor antigens can be retrieved, for example, from cancer/tumor epitope databases, e.g., as described in Vigneron et al. 2013, Cancer Immun. 13:15; URL: http://www.cancerimmunity.org/peptide/, or from the database “Tantigen” (TANTIGEN version 1.0, Dec. 1, 2009; developed by Bioinformatics Core at Cancer Vaccine Center, Dana-Farber Cancer Institute; URL: http://cvc.dfci.harvard.edu/tadb/).

The term “pharmaceutical composition” as used herein refers in particular to preparations which are in such a form as to permit biological activity of the active ingredient(s) to be unequivocally effective and which contain no additional component which would be toxic to subjects to which the said formulation would be administered. In some embodiments, the (pharmaceutical) composition does not contain a further active component (e.g., “active” regarding cancer treatment) in addition to the active components described herein below.

As used herein, the term “vaccine” refers to a biological preparation that provides innate and/or adaptive immunity, typically to a particular disease, preferably cancer. Thus, a vaccine supports in particular an innate and/or an adaptive immune response of the immune system of a subject to be treated. For example, the antigens or the multi-antigenic domain as described herein typically leads to or supports an adaptive immune response in the patient to be treated, and the TLR peptide agonist as described herein may lead to or support an innate immune response.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. Preferably, the subject or patient is a human.

Composition

In a first aspect the present invention provides a composition comprising
(i) at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2);
(ii) a nucleic acid encoding said tumor antigen, or the fragment or sequence variant thereof;
(iii) an antigen-presenting cell (APC) containing (i) or (ii); or
(iv) a T-cell expressing either a T-cell receptor or a CAR T-cell receptor targeting said tumor antigen.

The present inventors identified upstream open reading frames (ORFs), i.e., ORFs encoded in the 5′-UTR (5′-untranslated region) of tumor antigens, by analyzing selected Ribo-seq datasets, for example, from the RPFdb v.2.0 database (Wang et al, 2019, Nucleic Acids Res. 47, D230-D234). Thereby, the inventors have surprisingly identified peptides encoded in a 5′-upstream open reading frame (uORF) within the 5′-UTR of KRAS (Kirsten rat sarcoma viral oncogene homolog) mRNAs, peptides encoded in a 5′-upstream open reading frame (uORF) within the 5′-UTR of TPX2 (Targeting protein for Xklp2) mRNAs and peptides encoded in a 5′-upstream open reading frame (uORF) within the 5′-UTR of of AURKA (Aurora A kinase) mRNAs as tumor antigens. As demonstrated in the appended examples, such KRAS, TPX2 and AURKA transcripts were found to be strongly expressed in tumors in contrast to normal healthy tissue. The present inventors also found immunogenic epitopes within the KRAS-uORF1, TPX2-uORF1 and AURKA-uORF2 peptides and that epitopes derived from KRAS-uORF1, TPX2-uORF1 and AURKA-uORF2 peptides were presented by human dendritic cells in the context of MHC class I and/or MHC class II. In summary, these findings support the role of the KRAS-uORF1, TPX2-uORF1 and AURKA-uORF2 peptides as novel tumor-specific antigens useful in cancer immunotherapy.

Accordingly, the present invention provides a composition comprising at least one tumor antigen, which is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); or a fragment or sequence variant thereof. The tumor antigen is typically a peptide tumor antigen.

These tumor antigens (i.e., on the peptide level) are also referred to herein as “KRAS-uORF1”, “TPX2-uORF1” and “AURKA-uORF2”, respectively. The person skilled in the art would tell immediately from each context whether they are referred to a nucleotide or to a peptide.

In some embodiments, the composition comprises
a tumor antigen, or a fragment or sequence variant thereof, which is encoded in a 5′-upstream open reading frame (uORF) within the 5′ UTR of the KRAS mRNA (KRAS-uORF1),
a tumor antigen, or a fragment or sequence variant thereof, which is encoded in a 5′-upstream open reading frame (uORF) within the 5′ UTR of the TPX2 mRNA (TPX2-uORF1), and/or
a tumor antigen, or a fragment or sequence variant thereof, which is encoded in a 5′-upstream open reading frame (uORF) within the 5′ UTR of the AURKA mRNA (AURKA-uORF2).

In some embodiments, the composition comprises the tumor antigen encoded in the 5′-upstream open reading frame (uORF) within the 5′ UTR of the KRAS mRNA (KRAS-uORF1) comprises (or consists of) an amino acid sequence according to SEQ ID NO: 1 or 2. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2. The functionality as tumor antigen is preferably maintained in the fragment or sequence variant (e.g., in that the fragment or sequence variant contains at least one epitope).

In some embodiments, the composition comprises the tumor antigen encoded in the 5′-upstream open reading frame (uORF) within the 5′ UTR of the TPX2 mRNA (TPX2-uORF1) comprises (or consists of) an amino acid sequence according to SEQ ID NO: 3 or 4. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4. The functionality as tumor antigen is preferably maintained in the fragment or sequence variant (e.g., in that the fragment or sequence variant contains at least one epitope).

In some embodiments, the composition comprises the tumor antigen encoded in the 5′-upstream open reading frame (uORF) within the 5′ UTR of the AURKA mRNA (AURKA-uORF2) comprises (or consists of) an amino acid sequence according to SEQ ID NO: 5. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5. The functionality as tumor antigen is preferably maintained in the fragment or sequence variant (e.g., in that the fragment or sequence variant contains at least one epitope).

In addition to the tumor antigen(s) described above, the composition may further comprise at least one tumor antigen selected from the group consisting of CEACAM5 (or a fragment or sequence variant thereof as defined above), DUOXA2 (or a fragment or sequence variant thereof as defined above), and KRAS (or a fragment or sequence variant thereof as defined above). In contrast to the tumor antigens encoded in the 5′-upstream open reading frame (uORF) within a 5′ UTR as described above, the additional antigens CEACAM5, DUOXA2 and KRAS, as referred to herein, are tumor antigens, which are encoded in the (“classical”) coding sequence (CDS) of an mRNA (i.e., not in an “untranslated” region/UTR).

In some embodiments, the composition comprises a fragment of CEACAM5 (Carcinoembryonic antigen-related cell adhesion molecule 5), which may comprise (or consist of) an amino acid sequence according to SEQ ID NO: 6. The composition may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 6. The functionality as tumor antigen is preferably maintained in the fragment or sequence variant (e.g., in that the fragment or sequence variant contains at least one epitope).

In some embodiments, the composition comprises a fragment of CEACAM5, which may comprise (or consist of) an amino acid sequence according to SEQ ID NO: 7. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7. The functionality as tumor antigen is preferably maintained in the fragment or sequence variant (e.g., in that the fragment or sequence variant contains at least one epitope).

In some embodiments, the composition comprises a fragment of CEACAM5, which may comprise (or consist of) an amino acid sequence according to SEQ ID NO: 8. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 8. The functionality as tumor antigen is preferably maintained in the fragment or sequence variant (e.g., in that the fragment or sequence variant contains at least one epitope).

In some embodiments, the fragments of CEACAM5 as described above may be linked to each other (in particular as fusion peptide/protein). Accordingly, the composition may comprise a (fusion) peptide comprising (or consisting of) an amino acid sequence according to SEQ ID NO: 9. The composition may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9. The functionality as tumor antigen is preferably maintained in the fragment or sequence variant (e.g., in that the fragment or sequence variant contains at least one epitope).

In some embodiments, the composition comprises a fragment of DUOXA2 (Dual oxidase maturation factor 2), which may comprise (or consist of) an amino acid sequence according to SEQ ID NO: 10. The composition may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10. The functionality as tumor antigen is preferably maintained in the fragment or sequence variant (e.g., in that the fragment or sequence variant contains at least one epitope).

In some embodiments, the composition comprises a fragment of KRAS. As KRAS mutations are common in cancer, the fragment of KRAS preferably includes a mutation. The KRAS mutation may occur, for example at G12, G13 or Q61 of KRAS. Accordingly, the fragment of KRAS may include positions G12, G13 and/or Q61 of KRAS, preferably wherein at least one of the amino acid residues at these positions is substituted. Preferably, the fragment of KRAS includes a substitution at G12. Non-limiting examples of such substitutions include G12D, G12V, G12C, G12A and G12R. Preferably, the fragment of KRAS includes position G12 of KRAS with a G12D or G12V substitution. Accordingly, KRAS, or the fragment thereof, is preferably KRAS-G12D, or a fragment thereof, or KRAS-G12V, or a fragment thereof (wherein the fragment includes the G12 position of KRAS and the respective substitution).

In some embodiments, the composition comprises a fragment of KRAS, which may comprise (or consist of) an amino acid sequence according to SEQ ID NO: 11 or 12. The composition may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11. The composition may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12. The functionality as tumor antigen is preferably maintained in the fragment or sequence variant (e.g., in that the fragment or sequence variant contains at least one epitope).

Preferably, the composition comprises different tumor antigens, or fragments or variants thereof, selected from CEACAM5, DUOXA2, KRAS, KRAS-uORF1, TPX2-uORF1 and AURKA-uORF2. In some embodiments, the composition comprises (exactly or at least) two different tumor antigens, or fragments or variants thereof, selected from CEACAM5, DUOXA2, KRAS, KRAS-uORF1, TPX2-uORF1 and AURKA-uORF2. In some embodiments, the composition comprises (exactly or at least) three different tumor antigens, or fragments or variants thereof, selected from CEACAM5, DUOXA2, KRAS, KRAS-uORF1, TPX2-uORF1 and AURKA-uORF2. In some embodiments, the composition comprises (exactly or at least) four different tumor antigens, or fragments or variants thereof, selected from CEACAM5, DUOXA2, KRAS, KRAS-uORF1, TPX2-uORF1 and AURKA-uORF2. In some embodiments, the composition comprises (exactly or at least) five different tumor antigens, or fragments or variants thereof, selected from CEACAM5, DUOXA2, KRAS, KRAS-uORF1, TPX2-uORF1 and AURKA-uORF2. In some embodiments, the composition comprises all of the six different tumor antigens, or fragments or variants thereof, selected from CEACAM5, DUOXA2, KRAS, KRAS-uORF1, TPX2-uORF1 and AURKA-uORF2. A multi-antigenic vaccine will (i) avoid outgrowth of antigen-loss variants, (ii) target different tumor cells within a heterogeneous tumor mass and (iii) circumvent patient-to-patient tumor variability.

Preferably, the composition comprises different tumor antigens, or fragments or variants thereof, selected from CEACAM5, DUOXA2, KRAS-G12D, KRAS-uORF1, TPX2-uORF1 and AURKA-uORF2. In some embodiments, the composition comprises different tumor antigens, or fragments or variants thereof, selected from CEACAM5, DUOXA2, KRAS-G12V, KRAS-uORF1, TPX2-uORF1 and AURKA-uORF2. In some embodiments, the composition comprises different tumor antigens, or fragments or variants thereof, selected from CEACAM5, DUOXA2, KRAS-G12D, KRAS-G12V, KRAS-uORF1, TPX2-uORF1 and AURKA-uORF2.

Preferably, the tumor antigen selected from CEACAM5, DUOXA2, KRAS, KRAS-uORF1, TPX2-uORF1 and AURKA-uORF2, or the fragment or sequence variant thereof, comprises a CD4+ and/or a CD8+ epitope.

In some embodiments, the composition according to the present invention comprises at least one CD4⁺ epitope and at least one CD8⁺ epitope, which may be included in the same or different tumor antigens (or fragments or variants thereof). Tₕ cells (CD4⁺ T cells) play a central role in the anti-tumor immune response both in DC (dendritic cell) licensing and in the recruitment and maintenance of CTLs (CD8⁺ T cells, cytotoxic T lymphocytes) at the tumor site. Therefore, a composition according to the present invention comprising at least one CD4⁺ epitope and at least one CD8⁺ epitope provides an integrated immune response allowing simultaneous priming of CTLs and Tₕ cells and is thus preferable to immunity against only CD8⁺ or only CD4⁺ epitopes.

Preferably, the composition comprises (i) different tumor antigens, or fragments or variants thereof, selected from CEACAM5, DUOXA2, KRAS, KRAS-uORF1, TPX2-uORF1 and AURKA-uORF2; and (ii) different CD4⁺ epitopes and different CD8⁺ epitopes. Such a composition induces multi-epitopic CD8⁺ CTLs and CD4⁺ Tₕ cells to function synergistically to counter tumor cells and promote efficient anti-tumor immunity. Tₕ cells are also involved in the maintenance of long-lasting cellular immunity that was monitored after vaccination. Such a composition induces polyclonal, multi-epitopic immune responses and poly-functional CD8⁺ and CD4⁺ T cells, and thus efficacious anti-tumor activity.

In some embodiments, the composition comprises the tumor antigen comprises at least one of amino acid sequence, wherein the amino acid sequence is
an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11;
an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1, or an amino acid sequence according to SEQ ID NO: 2, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2;
an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3, or an amino acid sequence according to SEQ ID NO: 4, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4;
an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5;
an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9; and/or
an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10.

In some embodiments, the composition comprises
a tumor antigen comprising (or consisting of) an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11;
a tumor antigen comprising (or consisting of) an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
a tumor antigen comprising (or consisting of) an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1, or a tumor antigen comprising (or consisting of) an amino acid sequence according to SEQ ID NO: 2, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2;
a tumor antigen comprising (or consisting of) an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3, or a tumor antigen comprising (or consisting of) an amino acid sequence according to SEQ ID NO: 4, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4;
a tumor antigen comprising (or consisting of) an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5;
a tumor antigen comprising (or consisting of) an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9; and/or
a tumor antigen comprising (or consisting of) an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10.

It is understood that the tumor antigens (or the fragments or variants thereof) are preferably comprised in the composition as peptides, as described above. Alternatively (or additionally), the composition may comprise a nucleic acid encoding the tumor antigen (or the fragment or variant thereof), as described above.

Nucleic acids preferably comprise single stranded, double stranded or partially double stranded nucleic acids, preferably selected from genomic DNA, cDNA, RNA, siRNA, antisense DNA, antisense RNA, ribozyme, complimentary RNA/DNA sequences with or without expression elements, a mini-gene, gene fragments, regulatory elements, promoters, and combinations thereof. Further preferred examples of nucleic acid (molecules) and/or polynucleotides include, e.g., a recombinant polynucleotide, a vector, an oligonucleotide, an RNA molecule such as an rRNA, an mRNA, an miRNA, an siRNA, or a tRNA, or a DNA molecule as described above. It is thus preferred that the nucleic acid (molecule) is a DNA molecule or an RNA molecule; preferably selected from genomic DNA; cDNA; siRNA; rRNA; mRNA; antisense DNA; antisense RNA; ribozyme; complimentary RNA and/or DNA sequences; RNA and/or DNA sequences with or without expression elements, regulatory elements, and/or promoters; a vector; and combinations thereof.

In some embodiments, the composition comprises
a nucleic acid encoding KRAS-uORF1, the nucleic acid preferably comprising a polynucleotide sequence according to SEQ ID NO: 37, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 37;
a nucleic acid encoding TPX2-uORF1, the nucleic acid preferably comprising a polynucleotide sequence according to SEQ ID NO: 38, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 38;
a nucleic acid encoding AURKA-uORF2, the nucleic acid preferably comprising a polynucleotide sequence according to SEQ ID NO: 39, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 39;
a nucleic acid encoding a fragment of CEACAM5, the nucleic acid preferably comprising a polynucleotide sequence according to SEQ ID NO: 40, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 40;
a nucleic acid encoding a fragment of CEACAM5, the nucleic acid preferably comprising a polynucleotide sequence according to SEQ ID NO: 41, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 41;
a nucleic acid encoding a fragment of CEACAM5, the nucleic acid preferably comprising a polynucleotide sequence according to SEQ ID NO: 42, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 42;
a nucleic acid encoding a fragment of DUOXA2, the nucleic acid preferably comprising a polynucleotide sequence according to SEQ ID NO: 43, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 43;
a nucleic acid encoding a fragment of KRAS-G12D, the nucleic acid preferably comprising a polynucleotide sequence according to SEQ ID NO: 44, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 44; and/or
a nucleic acid encoding a fragment of KRAS-G12V, the nucleic acid preferably comprising a polynucleotide sequence according to SEQ ID NO: 45, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 45.

The composition may comprise one or more nucleic acid molecules encoding different tumor antigens (or the fragments or variants thereof), as described above, e.g. 2, 3, 4, 5 or all 6 tumor antigens (or the fragments or variants thereof) as described above. The different tumor antigens (or the fragments or variants thereof), as described above, may be encoded by the same nucleic acid molecule (e.g., in a polycistronic manner) or in different nucleic acid molecules.

Alternatively (or additionally), the composition may comprise an antigen-presenting cell (APC) containing (i) the tumor antigen (or the fragment or variant thereof) as described above or (ii) the nucleic acid, as described above, encoding the tumor antigen (or the fragment or variant thereof). In some embodiments, the antigen-presenting cell (APC) may be a dendritic cell (DC).

In some embodiments, the APC may be loaded with the tumor antigen (or the fragment or variant thereof) as described above, such that the APC can present one or more epitopes of the tumor antigen (or the fragment or variant thereof).

The composition may comprise different antigen-presenting cell(s), containing (i) different tumor antigens (or the fragments or variants thereof); or (ii) different nucleic acid molecules as described above. However, a single APC may also contain (i) different tumor antigens (or the fragments or variants thereof); or (ii) different nucleic acid molecules as described above.

Alternatively (or additionally), the composition may comprise a T-cell expressing either a T-cell receptor or a CAR T-cell receptor targeting the tumor antigens (or the fragments or variants thereof), as described above. In some embodiments, the composition may comprise different T-cells, each expressing a different T-cell receptor or CAR T-cell receptor targeting a different tumor antigen (or the fragments or variants thereof), as described above.

In general, the composition may be a pharmaceutical composition and/or a vaccine. In particular, such a composition is preferably a (pharmaceutical) composition which optionally comprises a pharmaceutically acceptable carrier and/or vehicle, or any excipient, buffer, stabilizer or other materials well known to those skilled in the art.

As a further ingredient, the (pharmaceutical) composition may in particular comprise a pharmaceutically acceptable carrier and/or vehicle. In the context of the present invention, a pharmaceutically acceptable carrier typically includes the liquid or non-liquid basis of the (pharmaceutical) composition. If the (pharmaceutical) composition is provided in liquid form, the carrier will typically be pyrogen-free water; isotonic saline or buffered (aqueous) solutions, e.g., phosphate, citrate etc. buffered solutions. Particularly for injection of the (pharmaceutical) composition, water or preferably a buffer, more preferably an aqueous buffer, may be used, containing a sodium salt, preferably at least 30 mM of a sodium salt, a calcium salt, preferably at least 0.05 mM of a calcium salt, and optionally a potassium salt, preferably at least 1 mM of a potassium salt. According to a preferred embodiment, the sodium, calcium and, optionally, potassium salts may occur in the form of their halogenides, e.g., chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer. According to a more preferred embodiment, the buffer suitable for injection purposes as defined above, may contain salts selected from sodium chloride (NaCl), calcium chloride (CaCl₂) and optionally potassium chloride (KCl), wherein further anions may be present additional to the chlorides. CaCl₂ can also be replaced by another salt like KCl. Typically, the salts in the injection buffer are present in a concentration of at least 30 mM sodium chloride (NaCl), at least 1 mM potassium chloride (KCl) and at least 0.05 mM calcium chloride (CaCl₂). The injection buffer may be hypertonic, isotonic or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media are e.g., liquids occurring in “in vivo” methods, such as blood, lymph, cytosolic liquids, or other body liquids, or e.g. liquids, which may be used as reference media in “in vitro” methods, such as common buffers or liquids. Such common buffers or liquids are known to a skilled person. Saline (0.9% NaCl) and Ringer-Lactate solution are particularly preferred as a liquid basis. In some embodiments, the (pharmaceutical) composition further comprises arginine, such as L-arginine.

In this context, prescription of treatment, e.g., decisions on dosage etc. when using the above (pharmaceutical) composition is typically within the responsibility of general practitioners and other medical doctors, and typically takes account of the disorder to be treated, the condition of the individual patient, the site of delivery, the method of administration and other factors known to practitioners. Accordingly, the (pharmaceutical) composition typically comprises a therapeutically effective amount of the active components (the tumor antigen(s)). The (pharmaceutical) composition may be used for human and also for veterinary medical purposes, preferably for human medical purposes, as a (pharmaceutical) composition in general or as a vaccine.

Examples of suitable adjuvants and/or immunomodulatory materials in the context of the present invention include MPL® (Corixa), aluminum-based minerals including aluminum compounds (generically called Alum), ASO1-4, MF59, CalciumPhosphate, Liposomes, Iscom, polyinosinic:polycytidylic acid (polylC), including its stabilized form poly-ICLC (Hiltonol), CpG oligodeoxynucleotides, Granulocyte-macrophage colony-stimulating factor (GM-CSF), lipopolysaccharide (LPS), Montanide, polylactide co-glycolide (PLG), Flagellin, Soap Bark tree saponins (QS21), amino alkyl glucosamide compounds (e.g. RC529), two component antibacterial peptides with synthetic oligodeoxynucleotides (e.g. IC31), Imiquimod, Resiquimod, Immunostimulatory sequences (ISS), monophosphoryl lipid A (MPLA), and Fibroblast-stimulating lipopeptide (FSL1).

Further materials as well as formulation processing techniques and the like, which are useful in the context of compositions, in particular pharmaceutical compositions and vaccines, or in the context of their preparation are known to the skilled artisan.

Peptide

In a further aspect the present invention provides a peptide comprising:
a) a cell penetrating peptide;
b) a multi-antigenic domain comprising at least one tumor antigen, or a fragment or sequence variant thereof,
   wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); and
c) a TLR peptide agonist.

In the peptide of the present invention, the components a)-c) are covalently linked, usually with a peptide bond. Accordingly, said peptide is a recombinant peptide (not occurring in nature) or a “fusion” peptide (wherein the components a)-c) are “fused” to each other).

Such a peptide provides simultaneous (i) stimulation of multi-epitopic cytotoxic T cell-mediated immunity, (ii) induction of Tₕ cells and (iii) promotion of immunological memory. Thereby, the peptide according to the present invention provides a potent vaccine, in particular having improved anti-tumor activity.

Multi-Antigenic Domain

The peptide of the invention comprises a multi-antigenic domain. As used herein the term “multi-antigenic domain” refers to a domain, such as a peptide, comprising at least two (e.g., 2, 3, 4, 5, 6, 7, 8, 9 or more) distinct antigens or fragments of at least two (e.g., 2, 3, 4, 5, 6, 7, 8, 9 or more) distinct antigens. In some embodiments, the multi-antigenic domain comprises three or more different antigens, or fragments thereof, in particular 3, 4, 5, 6, 7, 8, 9, 10 or more different antigens or fragments thereof. Preferably, the “multi-antigenic domain” comprises (fragments of) at least two (e.g., 2, 3, 4, 5, 6, 7, 8, 9 or more) distinct antigens, wherein each fragment or antigen comprises at least one antigenic epitope. More preferably, the “multi-antigenic domain” comprises (fragments of) at least two to eight distinct antigens, wherein each fragment/antigen comprises at least one antigenic epitope. Even more preferably, the “multi-antigenic domain” comprises (fragments of) five or six distinct antigens, wherein each fragment comprises at least one antigenic epitope.

In particular, the different (fragments of the) antigens are positioned consecutively in the multi-antigenic domain. In some embodiments, the different (fragments of the) antigens are linked to each other for example by a peptide spacer or linker (e.g., a GS-linker). The spacer or linker is usually neither component a), i.e., the cell penetrating peptide, nor component c), i.e. the TLR peptide agonist. In other embodiments, the different (fragments of the) antigens are directly linked to each other, i.e., without spacer or linker.

In some embodiments, each antigen, or fragment thereof, in the multi-antigenic domain comprises at least one CD4+ epitope and/or at least one CD8+ epitope, which may be included in the same or different tumor antigens (or fragments or variants thereof). Tₕ cells (CD4⁺ T cells) play a central role in the anti-tumor immune response both in DC (dendritic cell) licensing and in the recruitment and maintenance of CTLs (CD8⁺ T cells, cytotoxic T lymphocytes) at the tumor site. Therefore, a multi-antigenic domain comprising at least one CD4⁺ epitope and at least one CD8⁺ epitope provides an integrated immune response allowing simultaneous priming of CTLs and Tₕ cells and is thus preferable to immunity against only CD8⁺ or only CD4⁺ epitopes.

The multi-antigenic domain of the peptide comprises at least one tumor antigen, which is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); or a fragment or sequence variant thereof as defined above. The tumor antigen is typically a peptide tumor antigen. These tumor antigens (i.e., on the peptide level) are also referred to herein as “KRAS-uORF1”, “TPX2-uORF1” and “AURKA-uORF2”, respectively. The person skilled in the art would tell immediately from each context whether they are referred to a nucleotide and/or to a peptide.

In some embodiments, the multi-antigenic domain comprises
a tumor antigen, or a fragment or sequence variant thereof, which is encoded in a 5′-upstream open reading frame (uORF) within the 5′ UTR of the KRAS mRNA (KRAS-uORF1),
a tumor antigen, or a fragment or sequence variant thereof, which is encoded in a 5′-upstream open reading frame (uORF) within the 5′ UTR of the TPX2 mRNA (TPX2-uORF1) and/or
a tumor antigen, or a fragment or sequence variant thereof, which is encoded in a 5′-upstream open reading frame (uORF) within the 5′ UTR of the AURKA mRNA (AURKA-uORF2).

In some embodiments, the multi-antigenic domain comprises the tumor antigen encoded in the 5′-upstream open reading frame (uORF) within the 5′ UTR of the KRAS mRNA (KRAS-uORF1) comprises (or consists of) an amino acid sequence according to SEQ ID NO: 1 or 2. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 1 or 2, or a sequence variant thereof.

In some embodiments, the multi-antigenic domain comprises the tumor antigen encoded in the 5′-upstream open reading frame (uORF) within the 5′ UTR of the TPX2 mRNA (uORF-TPX2) comprises (or consists of) an amino acid sequence according to SEQ ID NO: 3 or 4. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 3 or 4, or a sequence variant thereof.

In some embodiments, the multi-antigenic domain comprises the tumor antigen encoded in the 5′-upstream open reading frame (uORF) within the 5′ UTR of the AURKA mRNA (AURKA-uORF2) comprises (or consists of) an amino acid sequence according to SEQ ID NO: 5. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 5, or a sequence variant thereof.

In addition to the tumor antigen(s) described above, the multi-antigenic domain may further comprise at least one tumor antigen selected from the group consisting of CEACAM5 (or a fragment or sequence variant thereof as defined above), DUOXA2 (or a fragment or sequence variant thereof as defined above), and KRAS (or a fragment or sequence variant thereof as defined above).

In some embodiments, the multi-antigenic domain comprises (a fragment of) CEACAM5 (Carcinoembryonic antigen-related cell adhesion molecule 5), which may comprise (or consist of) an amino acid sequence according to SEQ ID NO: 6. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 6. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 6, or a sequence variant thereof.

In some embodiments, the multi-antigenic domain comprises (a fragment of) CEACAM5, which may comprise (or consist of) an amino acid sequence according to SEQ ID NO: 7. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 7, or a sequence variant thereof.

In some embodiments, the multi-antigenic domain comprises (a fragment of) CEACAM5, which may comprise (or consist of) an amino acid sequence according to SEQ ID NO: 8. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 8. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 8, or a sequence variant thereof.

In some embodiments, the fragments of CEACAM5 as described above may be linked to each other (in particular as fusion peptide/protein). Accordingly, the multi-antigenic domain may comprise a (fusion) peptide comprising (or consisting of) an amino acid sequence according to SEQ ID NO: 9. The multi-antigenic domain may also comprise a fragment or sequence variant thereof as defined above, i.e. a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 9, or a sequence variant thereof.

In some embodiments, the multi-antigenic domain comprises (a fragment of) DUOXA2 (Dual oxidase maturation factor 2), which may comprise (or consist of) an amino acid sequence according to SEQ ID NO: 10. The multi-antigenic domain may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 10, or a sequence variant thereof.

In some embodiments, the multi-antigenic domain comprises (a fragment of) KRAS. As KRAS mutations are common in cancer, the (fragment of) KRAS preferably includes a mutation. The KRAS mutation may occur, for example at G12, G13 or Q61 of KRAS. Accordingly, the fragment of KRAS may include positions G12, G13 and/or Q61 of KRAS, preferably wherein at least one of the amino acid residues at these positions is substituted. Preferably, the fragment of KRAS includes a substitution at G12. Non-limiting examples of such substitutions include G12D, G12V, G12C, G12A and G12R. Preferably, the fragment of KRAS includes position G12 of KRAS with a G12D or G12V substitution. Accordingly, KRAS, or the fragment thereof, is preferably KRAS-G12D, or a fragment thereof, or KRAS-G12V, or a fragment thereof (wherein the fragment includes the G12 position of KRAS and the respective substitution).

In some embodiments, the multi-antigenic domain comprises a fragment of KRAS, which may comprise (or consist of) an amino acid sequence according to SEQ ID NO: 11 or 12. The multi-antigenic domain may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11. The multi-antigenic domain may also comprise a fragment or sequence variant thereof as defined above, i.e., a fragment having a minimum length of 8 amino acids or a sequence variant having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 11 or 12, or a sequence variant thereof.

Accordingly, the multi-antigenic domain may comprise at least one amino acid sequence selected from the group consisting of:
an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9;
an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11;
an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1, or an amino acid sequence according to SEQ ID NO: 2, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2;
an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3, or an amino acid sequence according to SEQ ID NO: 4, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4;
an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5; or
an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10.

Preferably, the multi-antigenic domain comprises different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) two different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) three different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) four different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) five different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises all of the six different tumor antigens, or fragments or variants thereof, selected from CEACAM5, DUOXA2, KRAS, KRAS-uORF1, TPX2-uORF1 and AURKA-uORF2. A multi-antigenic vaccine will (i) avoid outgrowth of antigen-loss variants, (ii) target different tumor cells within a heterogeneous tumor mass and (iii) circumvent patient-to-patient tumor variability.

Preferably, the multi-antigenic domain comprises different tumor antigens, or fragments or variants thereof, selected from CEACAM5, DUOXA2, KRAS-G12D, KRAS-uORF1, TPX2-uORF1 and AURKA-uORF2. In some embodiments, the composition comprises different tumor antigens, or fragments or variants thereof, selected from CEACAM5, DUOXA2, KRAS-G12V, KRAS-uORF1, TPX2-uORF1 and AURKA-uORF2. In some embodiments, the composition comprises different tumor antigens, or fragments or variants thereof, selected from CEACAM5, DUOXA2, KRAS-G12D, KRAS-G12V, KRAS-uORF1, TPX2-uORF1 and AURKA-uORF2.

Preferably, the tumor antigen selected from CEACAM5, DUOXA2, KRAS, KRAS-uORF1, TPX2-uORF1 and AURKA-uORF2, or the fragment or sequence variant thereof, comprises a CD4+ and/or a CD8+ epitope. Preferably, the multi-antigenic domain comprises (i) different tumor antigens, or fragments or variants thereof, selected from CEACAM5, DUOXA2, KRAS, KRAS-uORF1, TPX2-uORF1 and AURKA-uORF2; and (ii) different CD4⁺ epitopes and different CD8⁺ epitopes. A peptide comprising such a multi-antigenic domain induces multi-epitopic CD8⁺ CTLs and CD4⁺ Tₕ cells to function synergistically to counter tumor cells and promote efficient anti-tumor immunity. Tₕ cells are also involved in the maintenance of long-lasting cellular immunity that was monitored after vaccination. A peptide comprising such a multi-antigenic domain induces polyclonal, multi-epitopic immune responses and poly-functional CD8⁺ and CD4⁺ T cells, and thus efficacious anti-tumor activity.

In some embodiments, the multi-antigenic domain comprises, preferably in N- to C-terminal direction:
CEACAM5 or a fragment or sequence variant thereof,
KRAS or a fragment or sequence variant thereof,
KRAS-uORF1, or a fragment or sequence variant thereof,
TPX2-uORF1, or a fragment or sequence variant thereof,
AURKA-uORF2, or a fragment or sequence variant thereof, and
DUOXA2 or a fragment or sequence variant thereof.

Preferably, the multi-antigenic domain comprises, preferably in N- to C-terminal direction:
an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9;
an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11, and/or an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1, or an amino acid sequence according to SEQ ID NO: 2, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2;
an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3, or an amino acid sequence according to SEQ ID NO: 4, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4;
an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5; and
an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10.

More preferably, the multi-antigenic domain comprises (or consists of) an amino acid sequence according to SEQ ID NO: 13, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 13. It is also more preferred that the multi-antigenic domain comprises (or consists of) an amino acid sequence according to SEQ ID NO: 14, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 14.

Cell Penetrating Peptide

The cell penetrating peptide (CPP) allows for efficient delivery, i.e., transport and loading, in particular of the multi-antigenic domain, into the antigen presenting cells (APCs), in particular into the dendritic cells (DCs) and thus to the dendritic cells' antigen processing machinery.

The term “cell penetrating peptide” (“CPP”) is generally used to designate short peptides that are able to transport different types of cargo molecules across plasma membrane, and, thus, facilitate cellular uptake of various molecular cargoes (from nanosize particles to small chemical molecules and large fragments of DNA). “Cellular internalization” of the cargo molecule linked to the cell penetrating peptide generally means transport of the cargo molecule across the plasma membrane and thus entry of the cargo molecule into the cell. Depending on the particular case, the cargo molecule can, then, be released in the cytoplasm, directed to an intracellular organelle, or further presented at the cell surface. Cell penetrating ability, or internalization, of the cell penetrating peptide or of the peptide (comprising said cell penetrating peptide) according to the invention can be checked by standard methods known to one skilled in the art, including flow cytometry or fluorescence microscopy of live and fixed cells, immunocytochemistry of cells transduced with said peptide, and Western blot.

Usually, cell penetrating peptides (CPPs) have a length of 8 to 50 residues. Cell penetrating peptides typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Cell-Penetrating peptides are of different sizes, amino acid sequences, and charges, but all CPPs have a common characteristic that is the ability to translocate the plasma membrane and facilitate the delivery of various molecular cargoes to the cytoplasm or to an organelle of a cell. Cell-penetrating peptides have found numerous applications in medicine as drug delivery agents in the treatment of different diseases including cancer and virus inhibitors, as well as contrast agents for cell labeling and imaging. Various CPPs, which can be used as cell penetrating peptide, i.e., as component a), in the peptide according to the present invention, are also disclosed in the review: Milletti, F., Cell-penetrating peptides: classes, origin, and current landscape. Drug Discov Today 17 (15-16): 850-60, 2012.

Preferably, the cell penetrating peptide comprised in the peptide of the present invention has an amino acid sequence comprising or consisting of an amino acid sequence according to SEQ ID NO: 15, SEQ ID NO: 21, SEQ ID NO: 22, or SEQ ID NO: 23, or is a sequence variant thereof as described above.

In some embodiments, the cell penetrating peptide according to the invention has an amino acid sequence comprising or consisting of SEQ ID NO: 15, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 15.

In some embodiments, the cell penetrating peptide according to the invention has an amino acid sequence comprising or consisting of SEQ ID NO: 21, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 21.

In some embodiments, the cell penetrating peptide according to the invention has an amino acid sequence comprising or consisting of SEQ ID NO: 22, or sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 22.

In some embodiments, the cell penetrating peptide according to the invention has an amino acid sequence comprising or consisting of SEQ ID NO: 23, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 23.

These cell penetrating peptides are derived from the “ZEBRA” protein of the Epstein-Barr virus (EBV). “ZEBRA” (also known as Zta, Z, EB1, or BZLF1) generally refers to the basic-leucine zipper (bZIP) transcriptional activator of the Epstein-Barr virus (EBV). Without being bound to any theory, it is assumed that these cell penetrating peptides promote both, MHC class I and II restricted presentation of cargo antigens to CD8⁺ and CD4⁺ T cells, respectively. Accordingly, such a CPP can deliver multi-epitopic peptides to dendritic cells (DCs), and subsequently promote CTL and Th cell activation and anti-tumor function. Such a CPP can thus efficiently deliver the peptide according to the present invention to antigen presenting cells (APCs) and lead to multi-epitopic MHC class I and II restricted presentation.

Preferably, the cell penetrating peptide has an amino acid sequence comprising or consisting of an amino acid sequence according to SEQ ID NO: 15, or is a sequence variant thereof as described above.

It will be understood by one skilled in the art that the primary amino acid sequence of the cell penetrating peptide may further be post-translationally modified, such as by glycosylation or phosphorylation, without departing from the invention.

In certain embodiments, the cell penetrating peptide optionally further comprises, in addition to its amino acid sequence as described above, any one of, or any combination of:
(i) a nuclear localization signal (NLS). Such signals are well known to the skilled person and are described in Nair et al. (2003, Nucleic Acids Res. 31(1): 397-399); and/or
(ii) a targeting peptide, including tumor homing peptides such as those described in Kapoor et al. (2012, PLoS ONE 7(4): e35187).

Preferably, the cell penetrating peptide is (directly) linked to multi-antigenic domain and facilitates the cellular internalization of the multi-antigenic domain.

### TLR Peptide Agonist

In the peptide according to the present invention, the TLR peptide agonist allows an increased targeting of the vaccine towards dendritic cells along with self-adjuvancity. Physical linkage of a TLR peptide agonist to the CPP and the at least one antigen or antigenic epitope in the peptide according to the present invention provides an enhanced immune response by simultaneous stimulation of antigen presenting cells, in particular dendritic cells, that internalize, metabolize and display antigen(s).

As used herein, a “TLR peptide agonist” is an agonist of a Toll-like receptor (TLR), i.e., it binds to a TLR and activates the TLR, in particular to produce a biological response. Moreover, the TLR peptide agonist is a peptide as defined above. Preferably, the TLR peptide agonist comprises from 10 to 150 amino acids, more preferably from 15 to 130 amino acids, even more preferably from 20 to 120 amino acids, particularly preferably from 25 to 110 amino acids, and most preferably from 30 to 100 amino acids.

Toll like receptors (TLRs) are transmembrane proteins that are characterized by extracellular, transmembrane, and cytosolic domains. The extracellular domains containing leucine-rich repeats (LRRs) with horseshoe-like shapes are involved in recognition of common molecular patterns derived from diverse microbes. Toll like receptors include TLRs1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LP A, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly(LC). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B streptococcus heat labile soluble factor (GBS-F) or staphylococcus modulins. TLR7 may be activated by imidazoquinolines. TLR9 may be activated by unmethylated CpG DNA or chromatin-IgG complexes.

Preferably, the TLR peptide agonist comprised by the peptide according to the present invention is an agonist of TLR1, 2, 4, 5, 6, and/or 10. TLRs are expressed either on the cell surface (TLR1, 2, 4, 5, 6, and 10) or on membranes of intracellular organelles, such as endosomes (TLR3, 4, 7, 8, and 9). The natural ligands for the endosomal receptors turned out to be nucleic acid-based molecules (except for TLR4). The cell surface-expressed TLR1, 2, 4, 5, 6, and 10 recognize molecular patterns of extracellular microbes (Monie, T. P., Bryant, C. E., et al. 2009: Activating immunity: Lessons from the TLRs and NLRs. Trends Biochem. Sci. 34(11), 553-561). TLRs are expressed on several cell types but virtually all TLRs are expressed on DCs allowing these specialized cells to sense all possible pathogens and danger signals.

However, TLR2, 4, and 5 are constitutively expressed at the surface of DCs. Accordingly, the TLR peptide agonist comprised by the peptide according to the present invention is more preferably a peptide agonist of TLR2, TLR4 and/or TLR5. Even more preferably, the TLR peptide agonist is a TLR2 peptide agonist and/or a TLR4 peptide agonist.

TLR2 can detect a wide variety of ligands derived from bacteria, viruses, parasites, and fungi. TLR2 interacts with a broad and structurally diverse range of ligands, including molecules expressed by microbes and fungi.

A preferred TLR2 peptide agonist is annexin II or an immunomodulatory fragment thereof (having TLR agonist functionality), which is described in detail in WO 2012/048190 A1 and U.S. patent application Ser. No. 13/033,1546, in particular a TLR2 peptide agonist comprising an amino acid sequence according to SEQ ID NO: 7 of WO 2012/048190 A1 or fragments or variants thereof are preferred.

A TLR2 peptide agonist comprising or consisting of an amino acid sequence according to SEQ ID NO: 16 or 24; or a sequence variant thereof (which is, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 16, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 24) is preferred as component c), i.e. as the TLR peptide agonist, comprised by the peptide. Particularly preferably, the TLR peptide agonist has an amino acid sequence according to SEQ ID NO: 16.

A diversity of ligands interact with TLR4, including Monophosphoryl Upid A from Salmonella minnesota R595 (MPLA), lipopolysaccharides (LPS), mannans (Candida albicans), glycoinositolphospholipids (Trypanosoma), viral envelope proteins (RSV and MMTV) and endogenous antigens including fibrinogen and heat-shock proteins. Preferred TLR4 peptide agonists correspond to motifs that bind to TLR4, in particular (i) peptides mimicking the natural LPS ligand (RS01: Gln-Glu-Ile-Asn-Ser-Ser-Tyr and RS09: Ala-Pro-Pro-His-Ala-Leu-Ser) and (ii) Fibronectin derived peptides. The cellular glycoprotein Fibronectin (FN) has multiple isoforms generated from a single gene by alternative splicing of three exons. One of these isoforms is the extra domain A (EDA), which interacts with TLR4. Accordingly, suitable TLR peptide agonists comprise a fibronectin EDA domain or a fragment or variant thereof. Such suitable fibronectin EDA domains or a fragments or variants thereof are disclosed in EP 1913 954 B1, EP 2 476 440 A1, US 2009/0220532 A1, and WO 2011/101332 A1.

In some embodiments, the peptide according to the present invention comprises
a) a cell penetrating peptide having an amino acid sequence comprising or consisting of an amino acid sequence according to SEQ ID NO: 15, SEQ ID NO: 21, SEQ ID NO: 22, or SEQ ID NO: 23; or sequence variants thereof having at least 70% sequence identity;
b) a multi-antigenic domain comprising:
   a fragment of CEACAM5 having a minimum length of 8 amino acids,
   a fragment of DUOXA2 having a minimum length of 8 amino acids,
   a fragment of KRAS having a minimum length of 8 amino acids,
   KRAS-uORF1, or a fragment thereof having a minimum length of 8 amino acids,
   TPX2-uORF1, or a fragment thereof having a minimum length of 8 amino acids, and/or
   AURKA-uORF2, or a fragment thereof having a minimum length of 8 amino acids; and
c) a TLR peptide agonist, which is a TLR2 peptide agonist and/or a TLR4 peptide agonist.

The different components a), b) and c) of the peptide may be directly or indirectly linked (e.g., by a peptide bond). In other words, two components may directly adjoin or they may be linked by an additional component of the peptide, e.g. a peptide spacer or a linker. Preferably, a peptidic spacer consists of about 1, 2, 3, 4, 5, 6, 7, 8, 9, or 10 amino acids, more preferably of about 1, 2, 3, 4, or 5 amino acids. The amino acid sequence of the peptidic spacer may be identical to that of the N-terminal or C-terminal flanking region of any of the components a), b), or c). Alternatively a peptidic spacer can consist of non-natural amino acid sequences such as an amino acid sequence resulting from conservative amino acid substitutions of said natural flanking regions or sequences of known cleavage sites for proteases. In some embodiments, the peptidic spacer does not contain any Cys (C) residues. In some embodiments the linker sequence contains at least 20%, more preferably at least 40% and even more preferably at least 50% Gly or β-alanine residues. Appropriate linker sequences can be easily selected and prepared by a person skilled in the art. They may be composed of D and/or L amino acids.

Preferably, all three components a), b), and c) are linked via main-chain/main-chain linkage, thus resulting in a main chain of the peptide, which comprises the main chain of the cell penetrating peptide, the main chain of the multi-antigenic domain and the main chain of the TLR peptide agonist. In other words, the main chain of the cell penetrating peptide, the main chain of the multi-antigenic domain and the main chain of the TLR peptide agonist constitute the main chain of the peptide, optionally together with further components, for example linker(s) or spacer(s). Accordingly, the following arrangements of the components a), b), and c) are preferred, wherein said preferred arrangements are shown below in N-terminal→C-terminal direction of the main chain of the peptide and wherein all three components a), b), and c) are linked via main-chain/main-chain linkage and may be optionally linked by a linker, a spacer or another additional component:
(α) component a) (cell penetrating peptide)-component b) (multi-antigenic domain)-component c) (TLR peptide agonist);
(β) component c) (TLR peptide agonist)-component a) (cell penetrating peptide)-component b) (multi-antigenic domain);
(γ) component a) (cell penetrating peptide)-component c) (TLR peptide agonist)-component b) (multi-antigenic domain);
(δ) component c) (TLR peptide agonist)-component b) (multi-antigenic domain)-component a) (cell penetrating peptide);
(ε) component b) (multi-antigenic domain)-component a) (cell penetrating peptide)-component c) (TLR peptide agonist); or
(ζ) component b) (multi-antigenic domain)-component c) (TLR peptide agonist)-component a) (cell penetrating peptide).

Preferably, the multi-antigenic domain is positioned C-terminally of the cell penetrating peptide. More preferably, the components a), b) and c) are positioned in N-terminal to C-terminal direction of the main chain of said peptide in the order:
(α) component a)-component b)-component c); or
(s) component c)-component a)-component b),
wherein the components may be linked by a further component, in particular by a linker or a spacer.

A preferred exemplified peptide of the invention comprises, preferably in N-terminal to C-terminal direction:
a) a cell penetrating peptide having an amino acid sequence comprising or consisting of an amino acid sequence according to SEQ ID NO: 15, or sequence variants thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 15;
b) a multi-antigenic domain comprising
   an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9;
   an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11, and/or an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
   an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1;
   an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3;
   an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5; and
   an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10; and
c) a TLR peptide agonist having an amino acid sequence comprising or consisting of an amino acid sequence according to SEQ ID NO: 16, or sequence variants thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 16.

In some embodiments, the peptide of the invention may comprise or consist of an amino acid sequence according to SEQ ID NO: 17, SEQ ID NO: 18, SEQ ID NO: 46, or SEQ ID NO: 47; or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID SEQ ID NO: 17, SEQ ID NO: 18, SEQ ID NO: 46, or SEQ ID NO: 47. For example, the peptide of the invention may comprise or consist of an amino acid sequence according to SEQ ID NO: 17, SEQ ID NO: 18, SEQ ID NO: 46, or SEQ ID NO: 47. Preferably, the peptide of the invention may comprise or consist of an amino acid sequence according to SEQ ID NO: 17 or SEQ ID NO: 18; or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID SEQ ID NO: 17 or SEQ ID NO: 18. For example, the peptide of the invention may comprise or consist of an amino acid sequence according to SEQ ID NO: 17 or 18.

The present invention also provides a (pharmaceutical) composition comprising the peptide of the invention as described above. The (pharmaceutical) composition may comprise a pharmaceutically acceptable carrier and/or vehicle, or any excipient, buffer, stabilizer or other materials well known to those skilled in the art. Regarding such further ingredients, i.e. carriers, vehicles, excipients, buffers, stabilizers, adjuvants and the like, the detailed description of the composition according to the present invention above applies accordingly to the (pharmaceutical) composition comprising the peptide of the invention. The (pharmaceutical) composition typically comprises a therapeutically effective amount of the active components (the peptide). The (pharmaceutical) composition may be used for human and also for veterinary medical purposes, preferably for human medical purposes, as a (pharmaceutical) composition in general or as a vaccine.

The present invention also provides a nucleic acid (molecule) comprising a polynucleotide encoding the peptide of the invention as described above. Nucleic acids preferably comprise single stranded, double stranded or partially double stranded nucleic acids. Preferably, the nucleic acid (molecule) is selected from genomic DNA, cDNA, RNA, siRNA, mRNA, antisense DNA, antisense RNA, ribozyme, complimentary RNA/DNA sequences. It may (or may not) contain expression elements, a mini-gene, gene fragments, regulatory elements, promoters, and combinations thereof. Further preferred examples of nucleic acid (molecules) and/or polynucleotides include, e.g., a recombinant polynucleotide, a vector, an oligonucleotide, an RNA molecule such as an rRNA, an mRNA, an miRNA, an siRNA, or a tRNA, or a DNA molecule. It is thus preferred that the nucleic acid (molecule) is a DNA molecule or an RNA molecule; preferably selected from genomic DNA; cDNA; siRNA; rRNA; mRNA; antisense DNA; antisense RNA; ribozyme; complimentary RNA and/or DNA sequences; RNA and/or DNA sequences with or without expression elements, regulatory elements, and/or promoters; a vector; and combinations thereof. In some embodiments, the nucleic acid (molecule) may be a vector, e.g. an expression vector for expression of the peptide of the invention.

Vesicular Stomatitis Virus (VSV)

In a further aspect the present invention provides a recombinant vesicular stomatitis virus (VSV) encoding a multi-antigenic domain comprising at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2).

Vesicular stomatitis viruses (VSV) belong to rhabdoviruses (family rhabdoviridae) and are negative strand RNA-viruses. The genome of VSV is on a single molecule of negative-sense RNA molecule that encodes five major proteins: G protein (G), large protein (L), phosphoprotein (P), matrix protein (M) and nucleoprotein (N). In some embodiments, the vesicular stomatitis virus is Vesicular stomatitis Indiana virus (VSIV) or Vesicular stomatitis New Jersey virus (VSNJV).

Multi-Antigenic Domain

The VSV of the invention is a recombinant VSV, which comprises a multi-antigenic domain defined as above, in the context of the peptide. Accordingly, it refers to a domain, such as a peptide, comprising at least two (e.g., 2, 3, 4, 5, 6, 7, 8, 9 or more) distinct antigens or fragments of at least two (e.g., 2, 3, 4, 5, 6, 7, 8, 9 or more) distinct antigens. In some embodiments, the multi-antigenic domain comprises three or more different antigens, or fragments thereof, in particular 3, 4, 5, 6, 7, 8, 9, 10 or more different antigens or fragments thereof. Preferably, the “multi-antigenic domain” comprises (fragments of) at least two (e.g., 2, 3, 4, 5, 6, 7, 8, 9 or more) distinct antigens, wherein each fragment or antigen comprises at least one antigenic epitope. More preferably, the “multi-antigenic domain” comprises (fragments of) at least two to eight distinct antigens, wherein each fragment/antigen comprises at least one antigenic epitope. Even more preferably, the “multi-antigenic domain” comprises (fragments of) five or six distinct antigens, wherein each fragment comprises at least one antigenic epitope.

In particular, the different (fragments of the) antigens are positioned consecutively in the multi-antigenic domain. In some embodiments, the different (fragments of the) antigens are linked to each other for example by a peptide spacer or linker (e.g., a GS-linker). In other embodiments, the different (fragments of the) antigens are directly linked to each other, i.e., without spacer or linker.

In some embodiments, each antigen, or fragment thereof, in the multi-antigenic domain comprises at least one CD4+ epitope and/or at least one CD8+ epitope, which may be included in the same or different tumor antigens (or fragments or variants thereof). Tₕ cells (CD4⁺ T cells) play a central role in the anti-tumor immune response both in DC (dendritic cell) licensing and in the recruitment and maintenance of CTLs (CD8⁺ T cells, cytotoxic T lymphocytes) at the tumor site. Therefore, a multi-antigenic domain comprising at least one CD4⁺ epitope and at least one CD8⁺ epitope provides an integrated immune response allowing simultaneous priming of CTLs and Tₕ cells and is thus preferable to immunity against only CD8⁺ or only CD4⁺ epitopes.

The multi-antigenic domain of the VSV comprises at least one tumor antigen, which is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); or a fragment or sequence variant thereof as defined above. The tumor antigen is typically a peptide tumor antigen. These tumor antigens (i.e. on the peptide level) are also referred to herein as “KRAS-uORF1”, “TPX2-uORF1” and “AURKA-uORF2”, respectively. The person skilled in the art would tell immediately from each context whether they are referred to a nucleotide and/or to a peptide.

In some embodiments, the multi-antigenic domain comprises
a tumor antigen, or a fragment or sequence variant thereof, which is encoded in a 5′-upstream open reading frame (uORF) within the 5′ UTR of the KRAS mRNA (KRAS-uORF1),
a tumor antigen, or a fragment or sequence variant thereof, which is encoded in a 5′-upstream open reading frame (uORF) within the 5′ UTR of the TPX2 mRNA (TPX2-uORF1) and/or
a tumor antigen, or a fragment or sequence variant thereof, which is encoded in a 5′-upstream open reading frame (uORF) within the 5′ UTR of the AURKA mRNA (AURKA-uORF2).

In some embodiments, the multi-antigenic domain comprises the tumor antigen encoded in the 5′-upstream open reading frame (uORF) within the 5′ UTR of the KRAS mRNA (KRAS-uORF1) comprises (or consists of) an amino acid sequence according to SEQ ID NO: 1 or 2. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 1 or 2, or a sequence variant thereof.

In some embodiments, the multi-antigenic domain comprises the tumor antigen encoded in the 5′-upstream open reading frame (uORF) within the 5′ UTR of the TPX2 mRNA (uORF-TPX2) comprises (or consists of) an amino acid sequence according to SEQ ID NO: 3 or 4. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 3 or 4, or a sequence variant thereof.

In some embodiments, the multi-antigenic domain comprises the tumor antigen encoded in the 5′-upstream open reading frame (uORF) within the 5′ UTR of the AURKA mRNA (AURKA-uORF2) comprises (or consists of) an amino acid sequence according to SEQ ID NO: 5. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 5, or a sequence variant thereof.

In addition to the tumor antigen(s) described above, the multi-antigenic domain may further comprise at least one tumor antigen selected from the group consisting of CEACAM5 (or a fragment or sequence variant thereof as defined above), DUOXA2 (or a fragment or sequence variant thereof as defined above), and KRAS (or a fragment or sequence variant thereof as defined above).

In some embodiments, the multi-antigenic domain comprises (a fragment of) CEACAM5 (Carcinoembryonic antigen-related cell adhesion molecule 5), which may comprise (or consist of) an amino acid sequence according to SEQ ID NO: 6. The multi-antigenic domain may also comprise a fragment or sequence variant thereof as defined above, i.e., a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 6, or a sequence variant thereof.

In some embodiments, the multi-antigenic domain comprises (a fragment of) CEACAM5, which may comprise (or consist of) an amino acid sequence according to SEQ ID NO: 7. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 7, or a sequence variant thereof.

In some embodiments, the multi-antigenic domain comprises (a fragment of) CEACAM5, which may comprise (or consist of) an amino acid sequence according to SEQ ID NO: 8. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 8, or a sequence variant thereof.

In some embodiments, the fragments of CEACAM5 as described above may be linked to each other (in particular as fusion peptide/protein). Accordingly, the multi-antigenic domain may comprise a (fusion) peptide comprising (or consisting of) an amino acid sequence according to SEQ ID NO: 9. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 9, or a sequence variant thereof.

In some embodiments, the multi-antigenic domain comprises (a fragment of) DUOXA2 (Dual oxidase maturation factor 2), which may comprise (or consist of) an amino acid sequence according to SEQ ID NO: 10. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 10, or a sequence variant thereof.

In some embodiments, the multi-antigenic domain comprises (a fragment of) KRAS. As KRAS mutations are common in cancer, the (fragment of) KRAS preferably includes a mutation. The KRAS mutation may occur, for example at G12, G13 or Q61 of KRAS. Accordingly, the fragment of KRAS may include positions G12, G13 and/or Q61 of KRAS, preferably wherein at least one of the amino acid residues at these positions is substituted. Preferably, the fragment of KRAS includes a substitution at G12. Non-limiting examples of such substitutions include G12D, G12V, G12C, G12A and G12R. Preferably, the fragment of KRAS includes position G12 of KRAS with a G12D or G12V substitution. Accordingly, KRAS, or the fragment thereof, is preferably KRAS-G12D, or a fragment thereof, or KRAS-G12V, or a fragment thereof (wherein the fragment includes the G12 position of KRAS and the respective substitution).

In some embodiments, the multi-antigenic domain comprises a fragment of KRAS, which may comprise (or consist of) an amino acid sequence according to SEQ ID NO: 11 or 12. The multi-antigenic domain may also comprise a fragment or sequence variant thereof as defined above, i.e. a fragment having a minimum length of 8 amino acids or a sequence variant having at least 70% sequence identity. The functionality as tumor antigen is preferably maintained in the fragment or sequence variant (e.g., in that the fragment or sequence variant contains at least one epitope). Accordingly, the multi-antigenic domain preferably comprises an amino acid sequence according to SEQ ID NO: 11 or 12, or a sequence variant thereof.

Accordingly, the multi-antigenic domain may comprise at least one amino acid sequence selected from the group consisting of:
an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9;
an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11;
an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1, or an amino acid sequence according to SEQ ID NO: 2, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2;
an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 3, or an amino acid sequence according to SEQ ID NO: 4, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4;
an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5; or
an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10.

Preferably, the multi-antigenic domain comprises different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) two different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) three different tumor antigens, or fragments or variants thereof, selected from CEACAM5, DUOXA2, KRAS, KRAS-uORF1, TPX2-uORF1 and AURKA-uORF2.

In some embodiments, the multi-antigenic domain comprises (exactly or at least) four different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises (exactly or at least) five different tumor antigens, or fragments or variants thereof, selected from CEACAM5, DUOXA2, KRAS, KRAS-uORF1, TPX2-uORF1 and AURKA-uORF2. In some embodiments, the multi-antigenic domain comprises all of the six different tumor antigens, or fragments or variants thereof, selected from CEACAM5, DUOXA2, KRAS, KRAS-uORF1, TPX2-uORF1 and AURKA-uORF2. A multi-antigenic vaccine will (i) avoid outgrowth of antigen-loss variants, (ii) target different tumor cells within a heterogeneous tumor mass and (iii) circumvent patient-to-patient tumor variability.

Preferably, the tumor antigen selected from CEACAM5, DUOXA2, KRAS, KRAS-uORF1, TPX2-uORF1 and AURKA-uORF2, or the fragment or sequence variant thereof, comprises a CD4+ and/or a CD8+ epitope. Preferably, the multi-antigenic domain comprises (i) different tumor antigens, or fragments or variants thereof, selected from CEACAM5, DUOXA2, KRAS, KRAS-uORF1, TPX2-uORF1 and AURKA-uORF2; and (ii) different CD4 epitopes and different CD8⁺ epitopes.

In some embodiments, the multi-antigenic domain comprises, preferably in N- to C-terminal direction:
CEACAM5 or a fragment or sequence variant thereof,
KRAS or a fragment or sequence variant thereof,
KRAS-uORF1, or a fragment or sequence variant thereof,
TPX2-uORF1, or a fragment or sequence variant thereof,
AURKA-uORF2, or a fragment or sequence variant thereof, and
DUOXA2 or a fragment or sequence variant thereof.

Preferably, the multi-antigenic domain comprises, preferably in N- to C-terminal direction:
an amino acid sequence according to SEQ ID NO: 9, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 9;
an amino acid sequence according to SEQ ID NO: 11, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11, and/or an amino acid sequence according to SEQ ID NO: 12, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 12;
an amino acid sequence according to SEQ ID NO: 1, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 1, or an amino acid sequence according to SEQ ID NO: 2, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2;
an amino acid sequence according to SEQ ID NO: 3, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 2, or an amino acid sequence according to SEQ ID NO: 4, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 4;
an amino acid sequence according to SEQ ID NO: 5, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 5; and
an amino acid sequence according to SEQ ID NO: 10, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 10.

More preferably, the multi-antigenic domain comprises (or consists of) an amino acid sequence according to SEQ ID NO: 19 or 48, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 19, or 48. For example, the multi-antigenic domain comprises (or consists of) an amino acid sequence according to SEQ ID NO: 19 or 48, preferably SEQ ID No: 19.

VSV: Further Features

In some embodiments, the vesicular stomatitis virus (VSV) is an oncolytic vesicular stomatitis virus (VSV). As used herein, an “oncolytic” virus refers to a virus that preferentially infects and kills cancer cells. It is assumed that the infected cancer cells, which are destroyed by oncolysis, release new infectious virus particles or virions to targeting the “remaining” cancer/tumor. Oncolytic activity of the recombinant rhabdovirus of the invention may be tested in different assay systems known to the skilled artisan (an exemplary in vitro assay is described by Muik et al., Cancer Res., 74(13), 3567-78, 2014). It is to be understood that an oncolytic VSV may infect and lyse only specific types of cancer cells. Also, the oncolytic effect may vary depending on the type of cancer cells.

In some embodiments, the vesicular stomatitis virus (VSV) is replication-competent. The terms “replication competent” or “replication competent virus” as used herein refer to a virus which contains all the information within its genome to allow it to replicate within a cell. For example, replication competence of the recombinant vesicular stomatitis virus of the invention may be assessed according to the methods disclosed in Tani et al. JOURNAL OF VIROLOGY, August 2007, p. 8601-8612; or Garbutt et al. JOURNAL OF VIROLOGY, May 2004, p. 5458-5465. Preferably, the VSV is capable of replicating specifically within cancer cells. Preferably, the VSV specifically replicates in tumor cells, which have lost the ability to mount and respond to anti-viral innate immune responses (e.g., type-I IFN signaling). Viral replication in tumor cells leads to the cell death, and is assumed to result in the release of tumor associated antigens, local inflammation and the induction of anti-tumor immunity. On the other hand, abortive replication is preferred in “healthy cells”, such that the VSV may be rapidly excluded from normal tissues.

Preferably, in the genome of the VSV the gene coding for the glycoprotein G is replaced by the gene coding for the glycoprotein GP of Lymphocyte choriomeningitis virus (LCMV), wherein the glycoprotein GP of LCMV preferably comprises the amino acid sequence according to SEQ ID NO: 25, or a functional sequence variant thereof which is at least 80%, 85%, 86%, 87%, 88%, 89%, 90%, 91%, 92%, 93%, 94%, 95%, 96%, 97%, 98%, 99% identical thereto. Accordingly, a vesicular stomatitis virus (VSV) and, in particular, the VSV-GP (recombinant VSV with GP of LCMV, e.g. as disclosed in WO2010/040526) is preferred. Advantageous properties of the VSV-GP include one or more of the following: very potent and fast killer (<8 h); oncolytic virus; systemic application possible; significantly reduced neurotropism with abolished neurotoxicity; it reproduces lytically; strong activation of innate immunity; about 3 kb space for immunomodulatory cargos and antigens; recombinant with an arenavirus glycoprotein from the lymphocytic-choriomeningitis-virus (LCMV); favorable safety features in terms of reduced neurotoxicity and less sensitive to neutralizing antibody responses and complement destruction as compared to the wild type VSV (VSV-G); specifically replicates in tumor cells, which have lost the ability to mount and respond to anti-viral innate immune responses (e.g. type-I IFN signaling); abortive replication in “healthy cells” so is rapidly excluded from normal tissues; viral replication in tumor cells leads to the cell death, and assumed to result in the release of tumor associated antigens, local inflammation and the induction of anti-tumor immunity.

In some embodiments, the vesicular stomatitis virus (VSV) exhibits the following features:
it encodes in its genome the multi-antigenic domain as defined above,
it encodes in its genome a vesicular stomatitis virus nucleoprotein (N), large protein (L), phosphoprotein (P), and matrix protein (M),
the gene coding for the glycoprotein G of the vesicular stomatitis virus is replaced by the gene coding for the glycoprotein GP of lymphocyte choriomeningitis virus (LCMV), and/or
the glycoprotein G of the vesicular stomatitis virus is replaced by the glycoprotein GP of LCMV.

Preferably, the vesicular stomatitis virus (VSV) exhibits the following features:
it encodes in its genome a vesicular stomatitis virus nucleoprotein (N) comprising an amino acid as set forth in SEQ ID NO: 26, or a sequence variant thereof having at least 80%, 85%, 86%, 87%, 88%, 89%, 90%, 91%, 92%, 93%, 94%, 95%, 96%, 97%, 98%, 99% sequence identity;
it encodes in its genome a vesicular stomatitis virus phosphoprotein (P) comprising an amino acid as set forth in SEQ ID NO: 27, or a sequence variant thereof having at least 80%, 85%, 86%, 87%, 88%, 89%, 90%, 91%, 92%, 93%, 94%, 95%, 96%, 97%, 98%, 99% sequence identity;
it encodes in its genome a vesicular stomatitis virus large protein (L) comprising an amino acid as set forth in SEQ ID NO: 28, or a sequence variant thereof having at least 80%, 85%, 86%, 87%, 88%, 89%, 90%, 91%, 92%, 93%, 94%, 95%, 96%, 97%, 98%, 99% sequence identity;
it encodes in its genome a vesicular stomatitis virus matrix protein (M) comprising an amino acid as set forth in SEQ ID NO: 29, or a sequence variant thereof having at least 80%, 85%, 86%, 87%, 88%, 89%, 90%, 91%, 92%, 93%, 94%, 95%, 96%, 97%, 98%, 99% sequence identity;
it encodes in its genome a vesicular stomatitis virus the multi-antigenic domain as defined above,
the gene coding for the glycoprotein G of the vesicular stomatitis virus is replaced by the gene coding for the glycoprotein GP of lymphocyte choriomeningitis virus (LCMV), and/or
the glycoprotein G is replaced by the glycoprotein GP of LCMV.

More preferably, the vesicular stomatitis virus (VSV) according to any one of claims 43-85, and wherein it encodes in its genome
a phosphoprotein (P) comprising the amino acid sequence according to SEQ ID NO: 27, or a sequence variant thereof having at least 70% sequence identity,
a nucleoprotein (N) comprising the amino acid sequence according to SEQ ID NO: 26, or a sequence variant thereof having at least 70% sequence identity,
a matrix protein (M) comprising the amino acid sequence according to SEQ ID NO: 29, or a sequence variant thereof having at least 70% sequence identity,
a large protein (L) comprising the amino acid sequence according to SEQ ID NO: 28, or a sequence variant thereof having at least 70% sequence identity,
a glycoprotein (GP) comprising the amino acid sequence according to SEQ ID NO: 25, or a sequence variant thereof having at least 70% sequence identity more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 25, and
a multi-antigenic domain which comprises the amino acid sequence according to SEQ ID NO: 19, or a sequence variant thereof having at least 70% sequence identity more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 19, or the amino acid sequence according to SEQ ID NO: 48, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 48.

Accordingly, a vesicular stomatitis virus (VSV) comprising an RNA genome, which comprises (or consists of) an RNA sequence according to SEQ ID NO: 30, or a sequence variant thereof as defined above (e.g., a sequence variant having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity), is particularly preferred. In some embodiments, the vesicular stomatitis virus (VSV) comprises an RNA genome, which comprises (or consists of) an RNA sequence, which corresponds to the cDNA sequence according to SEQ ID NO: 49, or to a sequence variant thereof as defined above (e.g., a sequence variant having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity).

The present invention also provides a (pharmaceutical) composition comprising the VSV of the invention as described above. The (pharmaceutical) composition may further comprise a pharmaceutically acceptable carrier and/or vehicle, or any excipient, buffer, stabilizer or other materials well known to those skilled in the art. Regarding such further ingredients, i.e. carriers, vehicles, excipients, buffers, stabilizers, adjuvants and the like, the detailed description of the composition according to the present invention above applies accordingly to the (pharmaceutical) composition comprising the VSV of the invention. The (pharmaceutical) composition typically comprises a therapeutically effective amount of the active components (the VSV). The (pharmaceutical) composition may be used for human and also for veterinary medical purposes, preferably for human medical purposes, as a (pharmaceutical) composition in general or as a vaccine.

Vaccine, Kit and Combination

In a further aspect, the present invention also provides a vaccine comprising
(i) the composition according to the present invention as described above;
(ii) the peptide according to the present invention as described above; or
(iii) the vesicular stomatitis virus (VSV) according to the present invention as described above.

In general, a vaccine induces, supports or enhances an (innate and/or an adaptive) immune response of the immune system of a subject or patient to be treated. For example, the antigens, in particular the multi-antigenic domain, as described herein, typically lead to or support an adaptive immune response in the patient to be treated. The TLR peptide agonist of the peptide as described herein may lead to or support an innate immune response.

The vaccine may also comprise a pharmaceutically acceptable carrier, adjuvant, and/or vehicle as defined above for the (pharmaceutical) composition. In the specific context of the vaccine, the choice of a pharmaceutically acceptable carrier is determined in principle by the manner in which the vaccine is administered. The vaccine can be administered, for example, systemically or locally as described above. More preferably, vaccines may be administered by an intravenous, intratumoral, intradermal, subcutaneous, or intramuscular route. The vaccine is therefore preferably formulated in liquid (or sometimes in solid) form. The suitable amount of the vaccine to be administered can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices.

The vaccine can additionally contain one or more auxiliary substances in order to further increase its immunogenicity. In some embodiments, a synergistic action of the composition containing the antigen(s), the peptide and/or the VSV as described above and of an auxiliary substance, which may be optionally contained in the vaccine, may be achieved. Depending on the various types of auxiliary substances, various mechanisms can come into consideration in this respect. For example, compounds that permit the maturation of dendritic cells (DCs), for example lipopolysaccharides or TNF-alpha, form a first class of suitable auxiliary substances. In general, it is possible to use as auxiliary substance any agent that influences the immune system in the manner of a “danger signal” (LPS, GP96, etc.) or cytokines, such as GM-CSF, which allow an immune response produced by the composition containing the antigen(s), the peptide and/or the VSV as described above to be enhanced and/or influenced in a targeted manner. Particularly preferred auxiliary substances are cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta or TNF-alpha, growth factors, such as hGH.

Preferably, the vaccine comprises
(i) the peptide according to the present invention as described above; and
(ii) the vesicular stomatitis virus (VSV) according to the present invention as described above.

In a further aspect, the present invention also provides a kit comprising
(i) the peptide according to the present invention as described above; and
(ii) the vesicular stomatitis virus (VSV) according to the present invention as described above.

In a further aspect, the present invention also provides a combination comprising
(i) the peptide according to the present invention as described above; and
(ii) the vesicular stomatitis virus (VSV) according to the present invention as described above.

In general, in the vaccine, the kit and the combination of the invention, the different components (i) and (ii) may be provided (a) in the same composition or in the same container; or (b) in a (spatially) separate manner (e.g., in different compositions or different containers). Preferably, in the vaccine, the kit and the combination of the invention, (i) the peptide and (ii) the VSV are provided in a separate manner, e.g., in separate containers or separate compositions as described above. Separate provision of the different components (i) and (ii) enables separate administration of (i) the peptide and (ii) the VSV, e.g., via distinct routes, in distinct compositions and/or at different times/at a different schedule. In particular, separate provision of the different components (i) and (ii) enables the use of (i) the peptide and (ii) the VSV in a heterologous prime-boost regimen.

In the vaccine, the kit or the combination it is preferred that the multi-antigenic domain encoded in the (genome of the) vesicular stomatitis virus (VSV) comprises a (tumor) antigen, or a fragment or sequence variant thereof, which is also contained in the multi-antigenic domain of the peptide. It is also preferred that the multi-antigenic domain encoded in the (genome of the) vesicular stomatitis virus (VSV) comprises the amino acid sequences of each of the (tumor) antigens, fragment or sequence variant thereof, which is also contained in the multi-antigenic domain of the peptide. It is also preferred that the multi-antigenic domain of the peptide comprises an antigen, or a fragment thereof, which is contained in the multi-antigenic domain encoded in the genome of the vesicular stomatitis virus (VSV). It is further preferred that the multi-antigenic domain of the peptide comprises the amino acid sequences of each of the (tumor) antigens, fragment or sequence variant thereof, which is contained in the multi-antigenic domain encoded in the genome of the vesicular stomatitis virus (VSV).

This enables an advantageous heterologous prime-boost regimen. The principle of the heterologous prime-boost technology is to force the immune system to focus its response on a specific target antigen by avoiding an immune response against the antigen carrier or delivery system after sequential administration of the same antigen carrier or delivery system when used in homologous prime-boost regimens. In heterologous prime boost regimens the administration of the first immunogen primes cytotoxic T lymphocytes (CTLs) specific for the target antigen, however, priming also occurs for the antigen carrier or delivery system. By administering an unrelated second antigen carrier or delivery system, such as for example a viral vector during the “boost” phase, the immune system is faced with a large number of new antigens. As the second antigen carrier or delivery system also encodes/delivers the target antigen for which primed cells already exist, a strong memory response is raised by the immune system, expanding previously primed CTLs, which are specific for the target antigen.

Accordingly, the vaccine, the kit or the combination of the invention advantageously provide a target antigen in different contexts, namely, (i) contained in the peptide of the invention; and (ii) encoded in the VSV of the invention, which enables the use in a heterologous prime-boost regimen.

The corresponding antigen (tumor) antigen, or the fragment or sequence variant thereof, which is contained in the multi-antigenic domain of the peptide and in the multi-antigenic domain encoded in the VSV may be any antigen (or fragment or variant thereof) as described herein. Preferably, corresponding antigen (tumor) antigen, or the fragment or sequence variant thereof, is selected from the group consisting of:
a tumor antigen, or a fragment or sequence variant thereof, which is encoded in a 5′-upstream open reading frame (uORF) within the 5′ UTR of the KRAS mRNA (KRAS-uORF1),
a tumor antigen, or a fragment or sequence variant thereof, which is encoded in a 5′-upstream open reading frame (uORF) within the 5′ UTR of the TPX2 mRNA (TPX2-uORF1),
a tumor antigen, or a fragment or sequence variant thereof, which is encoded in a 5′-upstream open reading frame (uORF) within the 5′ UTR of the AURKA mRNA (AURKA-uORF2),
CEACAM5, or a fragment or sequence variant thereof as defined above,
DUOXA2, or a fragment or sequence variant thereof as defined above, and
KRAS, or a fragment or sequence variant thereof as defined above.

Preferably, the KRAS (or the fragment or sequence variant thereof) is a mutant KRAS as described above. More preferably, the KRAS (or the fragment or sequence variant thereof) is KRAS-G12D (or a fragment or sequence variant thereof) or KRAS-G12V (or a fragment or sequence variant thereof).

In some embodiments, the multi-antigenic domain of the peptide and the multi-antigenic domain encoded in the VSV each comprises a tumor antigen, or a fragment or sequence variant thereof, which is encoded in a 5′-upstream open reading frame (uORF) within the 5′ UTR of the KRAS mRNA (KRAS-uORF1).

In some embodiments, the multi-antigenic domain of the peptide and the multi-antigenic domain encoded in the VSV each comprises a tumor antigen, or a fragment or sequence variant thereof, which is encoded in a 5′-upstream open reading frame (uORF) within the 5′ UTR of the TPX2 mRNA (TPX2-uORF1).

In some embodiments, the multi-antigenic domain of the peptide and the multi-antigenic domain encoded in the VSV each comprises a tumor antigen, or a fragment or sequence variant thereof, which is encoded in a 5′-upstream open reading frame (uORF) within the 5′ UTR of the AURKA mRNA (AURKA-uORF2).

In some embodiments, the multi-antigenic domain of the peptide and the multi-antigenic domain encoded in the VSV each comprises CEACAM5, or a fragment or sequence variant thereof as defined above.

In some embodiments, the multi-antigenic domain of the peptide and the multi-antigenic domain encoded in the VSV each comprises DUOXA2, or a fragment or sequence variant thereof as defined above.

In some embodiments, the multi-antigenic domain of the peptide and the multi-antigenic domain encoded in the VSV each comprises KRAS, or a fragment or sequence variant thereof as defined above. Preferably, the KRAS (or the fragment or sequence variant thereof) is a mutant KRAS as described above. More preferably, the KRAS (or the fragment or sequence variant thereof) is KRAS-G12D (or a fragment or sequence variant thereof) or KRAS-G12V (or a fragment or sequence variant thereof).

Preferably, the multi-antigenic domain of the peptide and the multi-antigenic domain encoded in the VSV each comprises at least one amino acid sequence selected from the group consisting of:
an amino acid sequence according to SEQ ID NO: 9, or a sequence variant thereof having at least 70% sequence identity;
an amino acid sequence according to SEQ ID NO: 12, or a sequence variant thereof having at least 70% sequence identity;
an amino acid sequence according to SEQ ID NO: 11, or a sequence variant thereof having at least 70% sequence identity;
an amino acid sequence according to SEQ ID NO: 1 or 2, or a sequence variant thereof having at least 70% sequence identity;
an amino acid sequence according to SEQ ID NO: 3 or 4, or a sequence variant thereof having at least 70% sequence identity;
an amino acid sequence according to SEQ ID NO: 5, or a sequence variant thereof having at least 70% sequence identity; or
an amino acid sequence according to SEQ ID NO: 10, or a sequence variant thereof having at least 70% sequence identity.

More preferably, the multi-antigenic domain of the peptide and the multi-antigenic domain encoded in the VSV each comprises different (i.e., more than one) corresponding tumor antigens, or fragments or variants thereof, selected from CEACAM5, DUOXA2, KRAS, KRAS-uORF1, TPX2-uORF1 and AURKA-uORF2. In some embodiments, the multi-antigenic domain of the peptide and the multi-antigenic domain encoded in the VSV each comprises (exactly or at least) two corresponding tumor antigens, or corresponding fragments or variants thereof, selected from CEACAM5, DUOXA2, KRAS, KRAS-uORF1, TPX2-uORF1 and AURKA-uORF2. In some embodiments, the multi-antigenic domain of the peptide and the multi-antigenic domain encoded in the VSV each comprises (exactly or at least) three corresponding tumor antigens, or corresponding fragments or variants thereof, selected from CEACAM5, DUOXA2, KRAS, KRAS-uORF1, TPX2-uORF1 and AURKA-uORF2. In some embodiments, the multi-antigenic domain of the peptide and the multi-antigenic domain encoded in the VSV each comprises (exactly or at least) four corresponding tumor antigens, or corresponding fragments or variants thereof, selected from CEACAM5, DUOXA2, KRAS, KRAS-uORF1, TPX2-uORF1 and AURKA-uORF2. In some embodiments, the multi-antigenic domain of the peptide and the multi-antigenic domain encoded in the VSV each comprises (exactly or at least) five corresponding tumor antigens, or corresponding fragments or variants thereof, selected from CEACAM5, DUOXA2, KRAS, KRAS-uORF1, TPX2-uORF1 and AURKA-uORF2. In some embodiments, the multi-antigenic domain of the peptide and the multi-antigenic domain encoded in the VSV each comprises all of the six different corresponding tumor antigens, or corresponding fragments or variants thereof, selected from CEACAM5, DUOXA2, KRAS, KRAS-uORF1, TPX2-uORF1 and AURKA-uORF2.

Preferably, the multi-antigenic domain encoded in the genome of the vesicular stomatitis virus (VSV) comprises the amino acid sequences of each of the antigens, or fragments or sequence variants thereof, which are contained in the multi-antigenic domain of the peptide. In some embodiments, the multi-antigenic domain encoded in the genome of the vesicular stomatitis virus (VSV) includes one or more additional amino acid sequences, preferably of antigen(s), or fragment(s) or sequence variant(s) thereof, which are not included in the multi-antigenic domain of the peptide. In other embodiments, the multi-antigenic domain encoded in the genome of the vesicular stomatitis virus (VSV) consists of the amino acid sequences of each of the antigens, or fragments or sequence variants thereof, which are contained in the multi-antigenic domain of the peptide.

In some embodiments, the multi-antigenic domain of the peptide comprises the amino acid sequences of each of the antigens, or fragments thereof, of the multi-antigenic domain which is encoded in the genome of the vesicular stomatitis virus (VSV). In some embodiments, the multi-antigenic domain of the peptide includes one or more additional amino acid sequences, preferably of antigen(s), or fragment(s) or sequence variant(s) thereof, which are not included in the multi-antigenic domain encoded in the genome of the vesicular stomatitis virus (VSV). In other embodiments, the multi-antigenic domain of the peptide consists of the amino acid sequences of each of the antigens, or fragments or sequence variants thereof, which are contained in the multi-antigenic domain encoded in the genome of the vesicular stomatitis virus (VSV).

Preferably, the multi-antigenic domain encoded in the genome of the vesicular stomatitis virus (VSV) may comprise both (i) KRAS-G12D, or a fragment or sequence variant thereof; and (ii) KRAS-G12V, or a fragment or sequence variant thereof, while the multi-antigenic domain of the peptide may comprise either (i) KRAS-G12D, or a fragment or sequence variant thereof; or (ii) KRAS-G12V, or a fragment or sequence variant thereof. Accordingly, the multi-antigenic domain encoded in the genome of the vesicular stomatitis virus (VSV) may comprise both (i) an amino acid sequence according to SEQ ID NO: 11, or a sequence variant thereof having at least 70% sequence identity; and (ii) an amino acid sequence according to SEQ ID NO: 12, or a sequence variant thereof having at least 70% sequence identity, while the multi-antigenic domain of the peptide may comprise either (i) an amino acid sequence according to SEQ ID NO: 11, or a sequence variant thereof having at least 70% sequence identity; or (ii) an amino acid sequence according to SEQ ID NO: 12, or a sequence variant thereof having at least 70% sequence identity.

In some embodiments, the multi-antigenic domain of the peptide may comprise both (i) KRAS-G12D, or a fragment or sequence variant thereof; and (ii) KRAS-G12V, or a fragment or sequence variant thereof, while the multi-antigenic domain encoded in the genome of the vesicular stomatitis virus (VSV) may comprise either (i) KRAS-G12D, or a fragment or sequence variant thereof; or (ii) KRAS-G12V, or a fragment or sequence variant thereof. Accordingly, the multi-antigenic domain of the peptide may comprise both (i) an amino acid sequence according to SEQ ID NO: 11, or a sequence variant thereof having at least 70% sequence identity; and (ii) an amino acid sequence according to SEQ ID NO: 12, or a sequence variant thereof having at least 70% sequence identity, while the multi-antigenic domain encoded in the genome of the vesicular stomatitis virus (VSV) may comprise either (i) an amino acid sequence according to SEQ ID NO: 11, or a sequence variant thereof having at least 70% sequence identity; or (ii) an amino acid sequence according to SEQ ID NO: 12, or a sequence variant thereof having at least 70% sequence identity.

More preferably, in the vaccine, the kit or the combination of the invention,
the multi-antigenic domain of the peptide comprises an amino acid sequence according to SEQ ID NO: 13, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 13, or an amino acid sequence according to SEQ ID NO: 14, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 14, and
the multi-antigenic domain encoded in the genome of the vesicular stomatitis virus (VSV) comprises the amino acid sequence according to SEQ ID NO: 19, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 19 or the amino acid sequence according to SEQ ID NO: 48, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 48.

Still more preferably, in the vaccine, the kit or the combination of the invention,
the peptide comprises an amino acid sequence according to SEQ ID NO: 17, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 17, an amino acid sequence according to SEQ ID NO: 18, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 18, an amino acid sequence according to SEQ ID NO: 46, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 46, or an amino acid sequence according to SEQ ID NO: 47, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 47, and
the vesicular stomatitis virus (VSV) comprises an RNA genome comprising an RNA sequence according to SEQ ID NO: 30, or a sequence variant thereof having at least 70% sequence identity, more preferably one of at least 75%, 80%, 85%, 86%, 87%, 88%, 89%, 90%, 91%, 92%, 93%, 94%, 95%, 96%, 97%, 98%, or 99% sequence identity to the amino acid sequence of SEQ ID NO: 30.

In some embodiments of the vaccine, the kit or the combination of the invention, the vesicular stomatitis virus (VSV) comprises an RNA genome, which comprises (or consists of) an RNA sequence, which corresponds to the cDNA sequence according to SEQ ID NO: 49, or to a sequence variant thereof as defined above (e.g., a sequence variant having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity).

It is also preferred that the vaccine, the kit or the combination of the invention further comprises (iii) an inhibitor of the PD-1/PD-L1 pathway.

While, in general, the inhibitor of the PD-1/PD-L1 pathway may be provided (a) in the same composition or in the same container as any one of components (i) and (ii) (the peptide and the VSV); or (b) in a (spatially) separate manner (e.g. in different compositions or different containers), it is preferred in the vaccine, the kit and the combination of the invention, that the inhibitor of the PD-1/PD-L1 pathway is provided in a separate manner (separate from (i) the peptide and (ii) the VSV), e.g. in a separate container or in a separate composition. As used herein, the term “inhibitor” includes reduction, decrease, blocking and inhibition of the PD-1/PD-L1 pathway, including antagonists (and inverse agonists) of the PD-1/PD-L1 pathway.

In general, the PD-1/PD-L1 pathway is well-known in the art and described, for example, in Han Y, Liu D, Li L. PD-1/PD-L1 pathway: current researches in cancer. Am J Cancer Res. 2020 Mar. 1; 10(3):727-742. PD-1 (Programmed Cell Death Protein 1) and its ligand PD-L1 (Programmed Cell Death Ligand 1) are well-known for their role in cancer immune escape.

Non-limiting examples of inhibitors of the PD-1/PD-L1 pathway include pembrolizumab (anti-PD-1 antibody); nivolumab (anti-PD-1 antibody); pidilizumab (anti-PD-1 antibody); cemiplimab (anti-PD-1 antibody); PDR-001 (anti-PD-1 antibody); atezolizumab (anti-PD-L1 antibody); avelumab (anti-PD-L1 antibody); durvalumab (anti-PD-L1 antibody); and PD1-1, PD1-2 and PD1-3, as described herein (anti-PD-1 antibodies). Accordingly, the inhibitor of the PD-1/PD-L1 pathway may be selected from the group consisting of pembrolizumab; nivolumab; pidilizumab; cemiplimab; PDR-001; atezolizumab; avelumab; durvalumab, ezabenlimab, an antibody comprising a heavy chain comprising the amino acid sequence of SEQ ID NO: 31 and a light chain comprising the amino acid sequence of SEQ ID NO: 32 (PD1-1); an antibody comprising a heavy chain comprising the amino acid sequence of SEQ ID NO: 33 and a light chain comprising the amino acid sequence of SEQ ID NO: 34 (PD1-2); and an antibody comprising a heavy chain comprising the amino acid sequence of SEQ ID NO: 35 and a light chain comprising the amino acid sequence of SEQ ID NO: 36 (PD1-3).

Further PD-1 antagonists disclosed by Li et al. (supra), or known to be in clinical trials, such as AMP-224, MED10680 (AMP-514), BMS-936559, JS001-PD-1, SHR-1210, BMS-936559, TSR-042, JNJ-63723283, MED14736, MPDL3280A, may be used as alternative or in addition to the above mentioned antagonists. The INNs as used herein are meant to also encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

Antibodies PD1-1, PD1-2 and PD1-3 are antibodies comprising the following (full length) heavy chain and (full length) light chain sequences:
PD1-1: a heavy chain comprising the amino acid sequence of SEQ ID NO: 31 and a light chain comprising the amino acid sequence of SEQ ID NO: 32;
PD1-2: a heavy chain comprising the amino acid sequence of SEQ ID NO: 33 and a light chain comprising the amino acid sequence of SEQ ID NO: 34;
PD1-3: a heavy chain comprising the amino acid sequence of SEQ ID NO: 35 and a light chain comprising the amino acid sequence of SEQ ID NO: 36.

Preferably, the inhibitor of the PD-1/PD-L1 pathway may be selected from the group consisting of ezabenlimab; an antibody comprising a heavy chain comprising the amino acid sequence of SEQ ID NO: 31 and a light chain comprising the amino acid sequence of SEQ ID NO: 32 (PD1-1); an antibody comprising a heavy chain comprising the amino acid sequence of SEQ ID NO: 33 and a light chain comprising the amino acid sequence of SEQ ID NO: 34 (PD1-2); and an antibody comprising a heavy chain comprising the amino acid sequence of SEQ ID NO: 35 and a light chain comprising the amino acid sequence of SEQ ID NO: 36 (PD1-3).

Medical Treatment and Uses

The composition according to the present invention as described above, the peptide according to the present invention as described above, the vesicular stomatitis virus (VSV) according to the present invention as described above, the kit according to the present invention as described above, the vaccine according to the present invention as described above, or the combination according to the present invention as described above may be for use in medicine. Preferably, the composition according to the present invention as described above, the peptide according to the present invention as described above, the vesicular stomatitis virus (VSV) according to the present invention as described above, the kit according to the present invention as described above, the vaccine according to the present invention as described above, or the combination according to the present invention as described above are for use in the treatment of cancer.

Accordingly, the present invention also provides the use of the composition according to the present invention as described above, the peptide according to the present invention as described above, the vesicular stomatitis virus (VSV) according to the present invention as described above, the kit according to the present invention as described above, the vaccine according to the present invention as described above, or the combination according to the present invention as described above for the manufacture of a medicament for the treatment of cancer.

In a further aspect, the present invention provides a method for ameliorating, treating, or reducing (the risk of occurrence) of a cancer, or for inducing or enhancing an anti-tumor response, the method comprising administration of (an effective amount of) the composition according to the present invention as described above, the peptide according to the present invention as described above, the vesicular stomatitis virus (VSV) according to the present invention as described above, the kit according to the present invention as described above, the vaccine according to the present invention as described above, or the combination according to the present invention as described above to a subject in need thereof.

Preferably, the cancer (as referred to in the above methods and uses) is a cancer of the gastrointestinal tract (GI). Non-limiting examples of GI cancers include anal cancer; appendix cancer; cholangiocarcinoma/bile duct cancer, in particular extrahepatic bile duct cancer; gastrointestinal carcinoid tumor; colorectal cancer, in particular colon cancer, rectal cancer and metastatic colorectal cancer; esophageal cancer; gallbladder cancer; gastric (stomach) cancer; gastrointestinal stromal tumor (GIST), and pancreatic cancer, such as pancreatic ductal adenocarcinoma. Accordingly, the cancer is preferably selected from the group consisting of anal cancer; appendix cancer; cholangiocarcinoma/bile duct cancer, in particular extrahepatic bile duct cancer; gastrointestinal carcinoid tumor; colorectal cancer, in particular colon cancer, rectal cancer and metastatic colorectal cancer; esophageal cancer; gallbladder cancer; gastric (stomach) cancer; gastrointestinal stromal tumor (GIST), and pancreatic cancer, such as pancreatic ductal adenocarcinoma. More preferably, the cancer is selected from the group consisting of colon cancer, rectal cancer, colorectal cancer, metastatic colorectal cancer, pancreatic cancer, and pancreatic ductal adenocarcinoma.

For the envisaged medical treatment, in particular for the treatment of cancer (e.g., as described above), the peptide of the present invention as described above is preferably combined with the vesicular stomatitis virus (VSV) of the present invention as described above. Accordingly, the peptide for use as described above may be administered in combination with the vesicular stomatitis virus (VSV) of the present invention as described above. Moreover, the vesicular stomatitis virus (VSV) for use as described above may be administered in combination with the peptide of the present invention as described above. In such a combined use, the multi-antigenic domain of the peptide and the multi-antigenic domain encoded in the VSV are preferably adapted to each other as described in detail above, in the context of the vaccine, the kit and the combination of the invention comprising the peptide and the VSV.

In particular, a “combination” or “combined” use of the peptide and the VSV as described herein usually means that the treatment with the peptide as described herein is combined with the treatment with the VSV as described herein. In such a combined use of the peptide and the VSV of the present invention as described above (i.e., also for the respective vaccine, kit and combination of the invention as described above), each of the peptide and the vesicular stomatitis virus (VSV) are usually administered at least once. Preferably, the peptide and the vesicular stomatitis virus (VSV) are administered consecutively (not simultaneously).

However, even if one component (the peptide or the VSV) is not administered, e.g., at the same day as the other component, their treatment schedules are usually intertwined. Thereby, an advantageous heterologous prime-boost regimen, as described above, can be achieved. In particular, one component, e.g., the peptide, may be administered first (as “prime”), while the other component, e.g., the VSV, may be administered later (as “boost”); e.g., 1, 2, 3, 4, 5, 6, 7, 8, 9, 10, 11, 12, 13, 14, 15, 16, 17, 18, 19, 20 or more days or weeks after the “prime”. Thereby, the interval between prime and boost is usually selected such, that a strong immune response can be found. Preferably the peptide is administered prior to the administration of the vesicular stomatitis virus (VSV). Accordingly, the peptide may function as “prime” and the VSV as “boost” in the heterologous prime-boost regimen.

In some embodiments, the peptide is administered at least twice, preferably prior to and subsequent to the administration of the VSV. Accordingly, the peptide and the vesicular stomatitis virus (VSV) are administered in the order K-V-K, wherein “K” refers to a (single) administration of the peptide and “V” refers to a (single) administration of the VSV. In some embodiments, the peptide is administered repeatedly. For example, the peptide and the vesicular stomatitis virus (VSV) may be administered in the order K-V-K, K-V-K-K, K-V-K-K-K, or K-V-K-K-K-K (with “K” referring to the peptide and “V” to the VSV). In some embodiments, the treatment schedule comprises a single administration of the vesicular stomatitis virus (VSV), i.e., the VSV is administered only once.

In some embodiments, the peptide and the VSV are administered 2, 3, 4, 5, 6, 7, 8, 9, 10, 11, 12, 13, 14, 15, 16, 18, 19, 20, 21 days apart from each other, preferably from about 4 days to about 24 days apart from each other, more preferably from about 10 days to about 18 days apart from each other, even more preferably from about 12 days to about 16 days apart from each other. In some embodiments, the peptide is administered repeatedly, preferably once per every 2 to 6 weeks, more preferably once per every 3 to 5 weeks, even more preferably once every 4 weeks.

In some embodiments, the dose of the peptide may be from about 0.5 nmol to about 10 nmol. In some embodiments, the dose of the VSV may be from about 10⁶ TCID₅₀ to about 10¹¹ TCID₅₀. Preferably, the dose of the VSV may be from about 10⁷ TCID₅₀ to about 10¹¹ TCID₅₀, more preferably about 10⁸ TCID₅₀ to about 10⁹ TCID₅₀, or about 10⁷ TCID₅₀ to about 10⁸ TCID₅₀, more preferably, from about 1×10⁷ TCID₅₀ to about 1×10⁸ TCID₅₀.

In general, the peptide and the vesicular stomatitis virus (VSV) may be administered via the same route or different routes of administration. Preferably, the route of administration is selected from intravenous, subcutaneous, and intramuscular administration. Preferably, the peptide and the VSV are administered via distinct routes of administration. More preferably, the peptide is administered subcutaneously, and the vesicular stomatitis virus (VSV) are administered intravenously, or intratumorally, preferably, intravenously.

The (combined) use of the peptide and the VSV (also in the vaccine, kit and combination as described herein) may further comprise administration of an inhibitor of the PD-1/PD-L1 pathway, such as the inhibitors of the PD-1/PD-L1 pathway described above. Accordingly, the inhibitor of the PD-1/PD-L1 pathway may be selected from the group consisting of pembrolizumab; nivolumab; pidilizumab; cemiplimab; PDR-001; atezolizumab; avelumab; durvalumab, ezabenlimab, an antibody comprising a heavy chain comprising the amino acid sequence of SEQ ID NO: 31 and a light chain comprising the amino acid sequence of SEQ ID NO: 32 (PD1-1); an antibody comprising a heavy chain comprising the amino acid sequence of SEQ ID NO: 33 and a light chain comprising the amino acid sequence of SEQ ID NO: 34 (PD1-2); and an antibody comprising a heavy chain comprising the amino acid sequence of SEQ ID NO: 35 and a light chain comprising the amino acid sequence of SEQ ID NO: 36 (PD1-3).

The inhibitor of the PD-1/PD-L1 pathway is administered concomitantly, sequentially or alternately with the peptide or the vesicular stomatitis virus (VSV). In some embodiments, the inhibitor of the PD-1/PD-L1 pathway is administered at the same day and/or alternately with the peptide.

### EXAMPLES

### Example 1: Tumor Antigens Encoded within the 5′ UTR of KRAS mRNA (KRAS-uORF1). TPX2 mRNA (TPX2-uORF1) and AURKA mRNA (AURKA-uORF2)

Novel tumor antigens, which are encoded in open reading frames (ORFs) in the 5′ UTR of KRAS mRNA (KRAS-uORF1), TPX2 mRNA (TPX2-uORF1) and AURKA mRNA (AURKA-uORF2) were identified. The tumor antigens (peptides) are also referred to herein as “KRAS-uORF1 peptide” or “KRAS-uORF1”, “TPX2-uORF1 peptide” or “TPX2-uORF1”, and “AURKA-uORF2 peptide” or “AURKA-uORF2”, respectively. The respective tumor antigens have the amino acid sequences according to SEQ ID NO: 1 (KRAS-uORF1 peptide), SEQ ID NO: 3 (TPX2-uORF1 peptide) and SEQ ID NO: NO 5 (AURKA-uORF2 peptide).

Because uORFs are often characterized by unusual, non-canonical translation start sites, and KRAS-uORF1, TPX2-uORF1, and AURKA-uORF2 do not contain a conventional start codon, it was first verified that they could be translated in human cancer cell lines. To this end, the antigens were fused N-terminally to dTomato and the expression of the resulting fluorescent fusion proteins was confirmed by flow cytometry.

To examine tumor expression of KRAS-uORF1, TPX2-uORF1 and AURKA-uORF2 on RNA level, BaseScope detection of relevant transcripts was used. To this end, probes corresponding only to the KRAS-uORF1, TPX2-uORF1 and AURKA-uORF2 coding sequences themselves were employed. This allows to distinguish between those mRNA transcripts of a gene locus that encode the tumor antigen of interest and those, which do not (e.g., according to Ensembl, the AURKA gene locus encompasses 13 alternatively spliced transcripts with seven of these 13 transcripts encoding the full-length AURKA kinase, but only three encoding AURKA-uORF2).

In a first experiment transcription, i.e., the generation of relevant KRAS, TPX2, and AURKA mRNA transcripts, was assessed in (i) pancreatic and colorectal tumors, (ii) tumor-adjacent ‘healthy’ tissue; and (iii) healthy tissue from non-diseased organs. To this end, the stainings were performed on LEICA Bond Max©. The slides were evaluated for RNA quality by using positive control probe (housekeeping gene PPIB). After QC tests, the slides were tested using BaseScope probe: Basescope® 2.5 LS Probe BA-Hs-Aurka-Tu-1-1zz-stC1 Lot: 21175A (ACD #1084338-C1), Basescope® LS Probe BA-Hs-KRAS-1zz-stC1 Lot: 21175A (ACD #1084358) according to manufacturer's instruction. The slides were counterstained by hematoxylin and mounted using VectaMount mounting medium. A whole slide image was scanned at 40 times objective magnification using Nanozoomer S360© Digital Pathology Scanning System (Hamamatsu). The number of cancer cells with ≥1 red color signal (positive cancer cells) were evaluated as positive. The percentage of positive cancer cells was calculated with the aid of HALO software (Indica Lab) by a certified pathologist.

As the KRAS-uORF1, TPX2-uORF1, and AURKA-uORF2 peptide tumor antigens lack any known domains, it appears unlikely that they stably fold and accumulate in the cell. Without being bound to any theory, it is assumed that they undergo rapid proteasomal degradation, which may be advantageous, as this may lead to the production of antigenic epitopes. However, the expected ultra-low steady-state protein levels of these entities render them essentially undetectable with antibodies. To nevertheless assess their translation (i.e., their protein synthesis) in in human pancreatic (PDAC), gastric (GC), and colorectal (CRC) tumors (or tumor-adjacent healthy tissue), we resorted to the RiboSeq technique (Ingolia, N. T., Ghaemmaghami, S., Newman, J. R. S., and Weissman, J. S. (2009). Genome-Wide Analysis in Vivo of Translation with Nucleotide Resolution Using Ribosome Profiling. Science 324, 218-223). Briefly, at least 200 mg of fresh frozen tumor tissues as well as corresponding tumor-adjacent tissues derived from PDAC [n=12; only five of which with tumor-adjacent material], GC [n=5], and CRC [n=44] were purchased from a commercial provider (Indivumed GmbH, Hamburg) for RiboSeq library preparation, sequencing (NovaSeq6000; ≥ 200M reads/library), and bioinformatic analysis at a partner CRO (TB-Seq Inc, South San Francisco). Adapters were trimmed using Cutadapt before aligning them to the human genome assembly GRCh38.p13 using STAR. It was confirmed that ribosomal footprints matched the expected lengths spectrum. 28-36mers were considered for further analysis. Dataset quality and proper 3-nt periodicity was validated by assessing metagene read distributions around start/stop codons. To quantify AURKA-uORF2, TPX2-uORF1, and KRAS-uORF1 translation, the numbers of relevant in-frame reads (multiplied by 10⁷) were normalized for ORF length (under the assumption that translation is driven by the innermost RiboSeq-supported start codon) and sample library size (i.e., the sum of all trimmed, mappable reads post removal of rRNA- and tRNA-derived sequences) to allow comparison across samples.

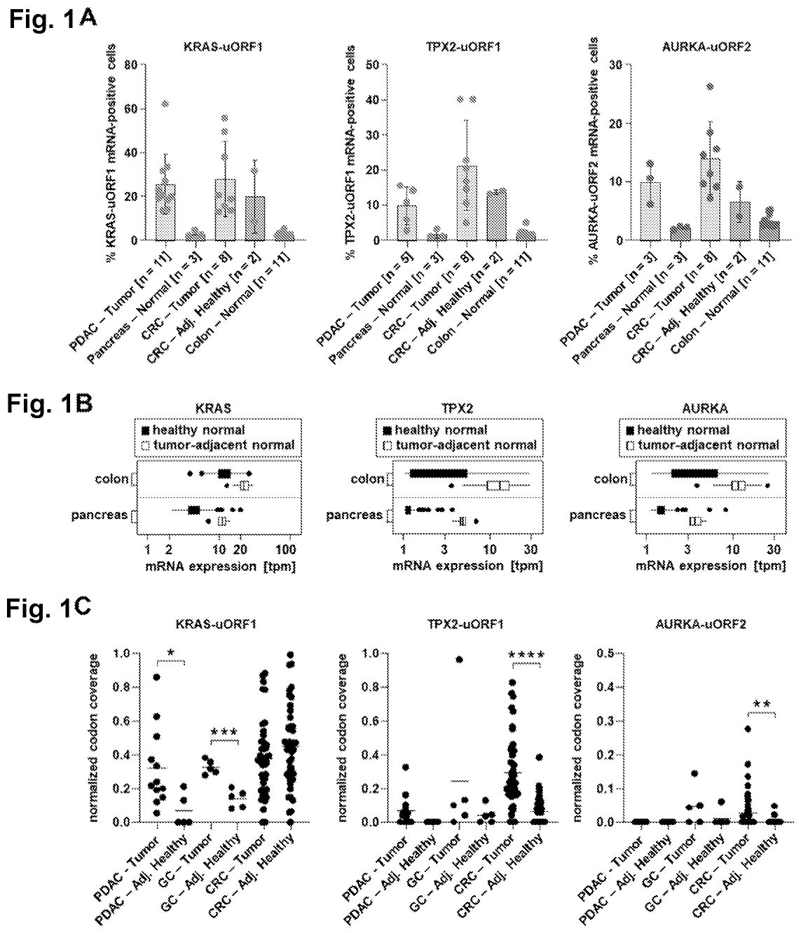

Results are shown in FIG. 1 with KRAS-uORF1-, TPX2-uORF1- and AURKA-uORF2-encoding transcript expression (A), RNA-Seq-based expression of KRAS, TPX2, and AURKA transcripts in healthy normal colon/pancreas versus tumor-adjacent colon/pancreas tissue (B) and translation of the uORFs of interest in human pancreatic (PDAC), gastric (GC), and colorectal (CRC) tumors or tumor-adjacent healthy tissue (C).

These data demonstrated strong expression of relevant KRAS, TPX2, and AURKA transcripts in pancreatic and colorectal tumors (FIG. 1A), while normal healthy tissue from colon, pancreas and other organs showed only low signal above background (FIG. 1A). This underscores that the encoded tumor antigens are likely targetable in these cancers. Tumor-adjacent ‘healthy’ tissue-unlike truly healthy tissue from non-diseased organs—displayed an intermediate transcript signal. This trend is also seen in RNASeq-based TCGA/GTEx datasets (FIG. 1B) and points to transcriptional dysregulation spreading from the tumor site into the immediately neighboring areas. Without being bound to any theory, it is thus assumed that tumor-adjacent healthy tissue might not always be fully representative of truly healthy tissue.

FIG. 1C shows that KRAS-uORF1, TPX2-uORF1, and AURKA-uORF2 peptide tumor antigens are expressed in tumors, albeit to various degrees in different indications including pancreatic, gastric, and colorectal cancer. In general, low or no synthesis was observed in healthy tissue immediately bordering the tumor (FIG. 1C). Without being bound to any theory, it is assumed that the KRAS-uORF1, TPX2-uORF1, and AURKA-uORF2 translation is not detected in most tumor-adjacent healthy tissues, even though BaseScope (FIG. 1A) and RNASeq (FIG. 1B) suggest the expression of corresponding mRNA transcripts, because of tumor-selective up-regulation of upstream ORF translation, which may be linked to the process of tumorigenesis itself. Thus, for KRAS-uORF1, TPX2-uORF1, and AURKA-uORF2, cancer-specific translation may add an additional layer of tumor specificity on top of largely cancer-specific transcription.

### Example 2: Expression Profiles of Additional Tumor Antigens CEACAM5 and DUOXA2

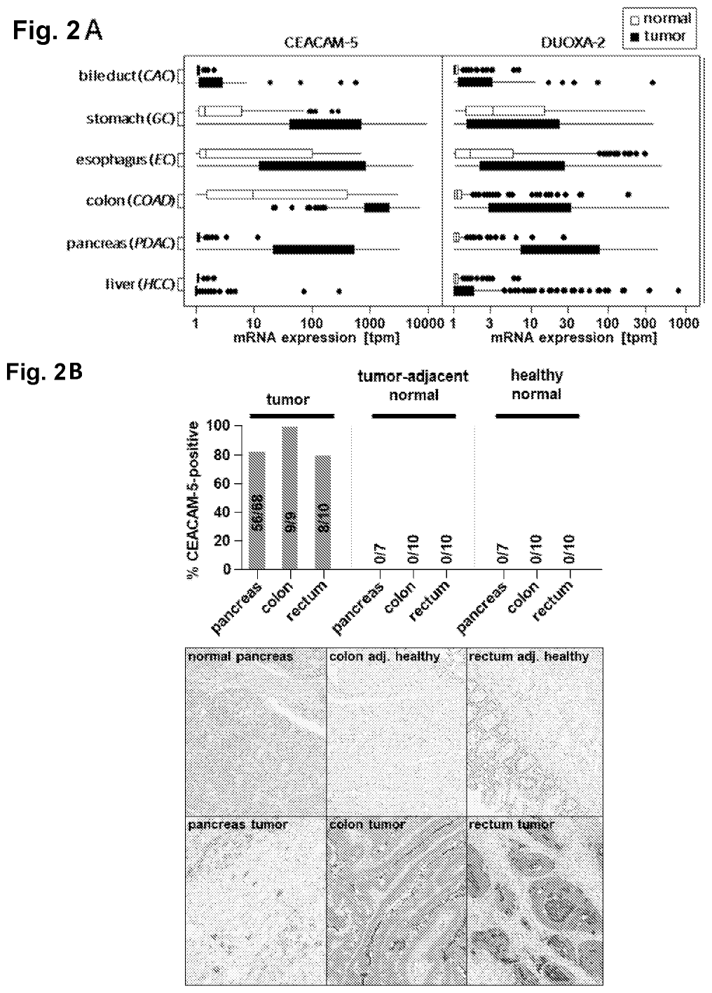

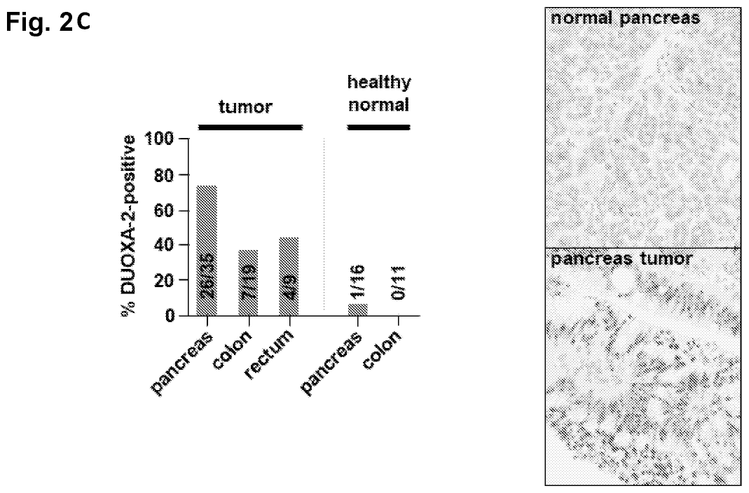

To select additional tumor antigens of interest, mRNA expression of CEACAM5 and DUOXA2 data were extracted from the following public databases: the Cancer Genome Atlas (TCGA) and the Genotype-Tissue Expression (GTEx). Based on the tumor-selective expression according to public RNASeq (FIG. 2A), CEACAM5 and DUOXA2 were selected.

Subsequently, the expression profile of CEACAM5 and DUOXA2 was experimentally validated in human tumor as well as non-tumor GI tract tissue. To measure CEACAM5 expression, Formalin-Fixed and Paraffin Embedded (FFPE) tissue arrays were purchased from a commercial provider (Indivumed GmbH, Hamburg). After QC tests, CEACAM5 staining was performed by ImmunoHistoChemistry on using CEACAM5-specific antibody (Dako M707229-2). The slides were counterstained by hematoxylin and mounted using VectaMount mounting medium. A whole slide image was scanned at 40 times objective magnification using Nanozoomer S360© Digital Pathology Scanning System (Hamamatsu). The number of cancer cells with ≥1 red color signal (positive cancer cells) were evaluated as positive. The percentage of positive cancer cells was calculated with the aid of HALO software (Indica Lab) by a certified pathologist.

Further, DUOXA2 expression was examined in GI tract tissue. To this end, Formalin-Fixed and Paraffin Embedded (FFPE) tissue arrays were purchased from a commercial provider (Indivumed GmbH, Hamburg). The slides were evaluated for RNA quality by using positive control probe (housekeeping gene PPIB). After QC tests, DUOXA2 transcript expression was performed by using RNAscope probe for DUOXA2 (Bio-Techne HD-RM-000619). according to manufacturer's instruction. The slides were counterstained by hematoxylin and mounted using VectaMount mounting medium. A whole slide image was scanned at 40 times objective magnification using Nanozoomer S360© Digital Pathology Scanning System (Hamamatsu). The number of cancer cells with ≥1 red color signal (positive cancer cells) were evaluated as positive. The percentage of positive cancer cells was calculated with the aid of HALO software (Indica Lab) by a certified pathologist.

Results are shown in FIG. 2. Specifically, CEACAM5 was detected by immunohistochemistry in 80-100% of pancreatic and colorectal cancers, but not in completely healthy or tumor-adjacent healthy tissue (FIG. 2B). DUOXA2 expression was solidly detected in 37-74% of pancreatic and colorectal cancers, while expression in corresponding healthy tissues was rare (e.g., only one out of 16 pancreatic cancers) (FIG. 2C).

### Example 3: Immunogenicity of KRAS-uORF1, TPX2-uORF1, AURKA-uORF2. CEACAM5, DUOXA2 and KRAS-G12D/V Antigens

In addition to the tumor antigens of Examples 1 and 2 above, a further tumor antigen of interest, namely KRAS, in particular KRAS-G12D/V, was included in the further analysis. Oncogenic driver mutations KRAS-G12D/V are generally not found outside tumors.

In a first step, co-expression on the mRNA level of mutant KRAS-G12D/V with CEACAM5 and DUOXA2 in pancreatic (PDAC) and colorectal (CRC) cancer (based on various RNASeq-based datasets including TCGA).

In addition, MHC class I epitopes for KRAS-uORF1, TPX2-uORF1, AURKA-uORF2, as well as for DUOXA2 were predicted using the NetMHCpan 4.1 prediction algorithm in order to identify peptides that bind to specific HLA class I alleles. A subset of these peptides and corresponding HLA allele-expressing donor PBMCs were then used for in vitro priming experiments to determine immunogenicity. To this end, dendritic cells were isolated from these PBMCs and pulsed with selected peptides, before they were used for the stimulation of autologous CD8 T cells, whose response was measured via IFNg ELISpot assays.

Next, the immunogenicity of KRAS-G12D/V was analyzed using two independent healthy peripheral blood mononuclear cells (PBMC) donors. To this end, CD8+ T cells were isolated from healthy donor peripheral blood mononuclear cells (PBMCs) by magnetic cell sorting using CD8 MicroBeads and columns (Miltenyi Biotec, Bergisch Gladbach, Germany). Monocytes were isolated from CD8+ depleted PBMCs by plastic adherence and subsequently treated with cytokines for generation and maturation of dendritic cells (DCs, antigen presenting cells).). Selection of donors occurred due to their expression of specific HLA alleles that are predicted for presentation of the respective peptide by DCs. CD8+ T cells were primed at least 3 times by peptide-loaded DCs before their specific immune response against a peptide was tested. A priming cycle included a co-culture of CD8+ T cells and peptide-loaded DCs for 7-10 days. After 3 priming cycles, CD8 T cells were plated in an ELISpot assay (Immunospot, Shaker Heights, OH 44122) for assessing their capacity to secrete IFN-γ after overnight stimulation with tumor cell lines or peptide-loaded DCs. Spots were counted using ELISPOT reader.

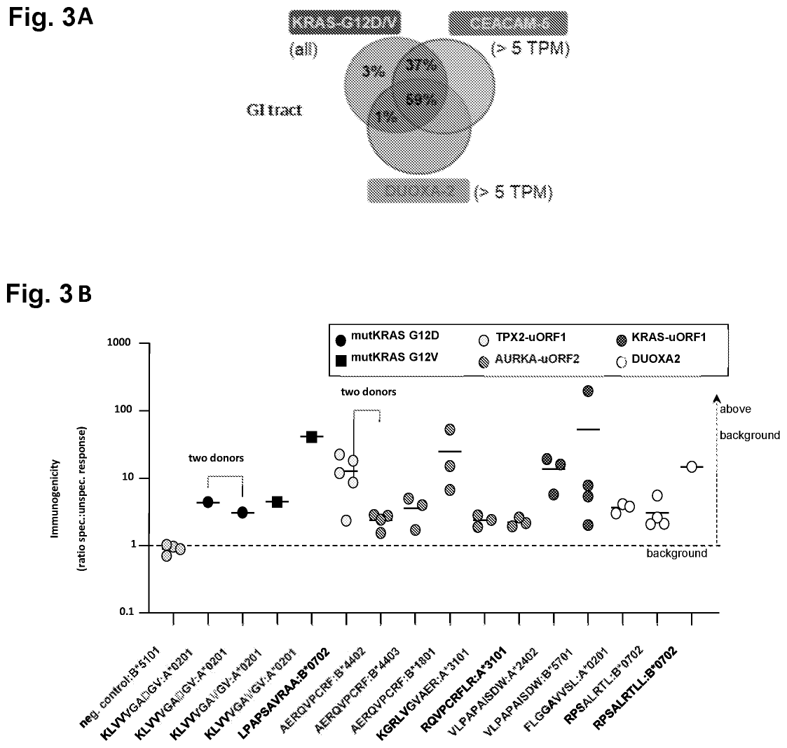

Results are shown in FIG. 3. As shown in FIG. 3A, co-expression analysis on the mRNA level of mutant KRAS-G12D/V with CEACAM5 and DUOXA2 in pancreatic (PDAC) and colorectal (CRC) cancer revealed that 59% of KRAS-G12D/V-mutant PDAC/CRC tumors express both CEACAM5 and DUOXA2 transcripts at >5 TPM. FIG. 3B shows the immunogenicity of epitopes derived from KRAS-uORF1, TPX2-uORF1, AURKA-uORF2, KRAS-G12D/V as well as for the DUOXA2. All of the tested peptides were found to be immunogenic (FIG. 3B). This suggests that cognate T cells will likely be available for activation in cancer patients.

### Example 4: Design and Evaluation of Peptides Comprising a Cell-Penetrating Peptide, a Multi-Antigenic Domain and a TLR Agonist

Next, peptides comprising (i) a cell-penetrating peptide, (ii) a multi-antigenic domain and (iii) a TLR agonist were designed, essentially as described in WO2016/146260. To this end, two different multi-antigenic domains were designed, which comprise either (immunogenic) fragments of KRAS-uORF1, TPX2-uORF1, AURKA-uORF2, KRAS-G12D, CEACAM5 and DUOXA2; or (immunogenic) fragments of KRAS-uORF1, TPX2-uORF1, AURKA-uORF2, KRAS-G12V, CEACAM5 and DUOXA2.

The multi-antigenic domain of both constructs comprises the following antigenic fragments of CEACAM5:

CEACAM5-1
[SEQ ID NO: 6]
NRTLTLFNVTRNDARAYVSGIQNSVSANRSDPVTLDVL
CEACAM5-2
[SEQ ID NO: 7]
PDSSYLSGANLNLSCHSAS
CEACAM5-3
[SEQ ID NO: 8]
PQYSWRINGIPQQHTQVLFIAKITPNNNGTYACFVSNLATGRNNSIVKSI
TVSASGTSPGLSA

Based on the three different fragments, a CEACAM5 fusion construct of the following sequence was designed for the multi-antigenic domain of both constructs:

[SEQ ID NO: 9]
NRTLTLFNVTRNDARAYVSGIQNSVSANRSDPVTLDVLPDSSYLSGANLN
LSCHSASPQYSWRINGIPQQHTQVLFIAKITPNNNGTYACFVSNLATGRN
NSIVKSITVSASGTSPGLSA

In addition, the following antigens or antigenic fragments were included in the multi-antigenic domain of both constructs:

| | DUOXA2: |
|---|---|
| [SEQ ID NO: 10] | |
| | TGVLSLFLGGAVVSLQYVRPSALRTLLDQSAKD |
| | KRAS-uORF1: |
| [SEQ ID NO: 1] | |
| | VAAARPVLPAPAISDWERARRRH |
| | TPX2-uORF1: |
| [SEQ ID NO: 3] | |
| | VGRKEGAARARVSLLPEFGTAEVHLPAPSAVRAARPGF |
| | AURKA-uORF2: |
| [SEQ ID NO: 5] | |
| | GGDKGRLVGVAERQVPCRFLRP |

The multi-antigenic domain of both constructs differs in that one comprises a fragment of KRAS-G12D (SEQ ID NO: 11), while the other comprises a fragment of KRAS-G12V (SEQ ID NO: 12):

| | KRAS-G12D: |
|---|---|
| (SEQ ID NO: 11) | |
| | TEYKLVVVGADGVGKSALTIQLIQ |
| | KRAS-G12V: |
| (SEQ ID NO: 12) | |
| | TEYKLVVVGAVGVGKSALTIQLIQ |

The two different peptide constructs are referred to as “ATP150” (comprising KRAS-G12D) and “ATP152” (comprising KRAS-G12V). They comprise the following multi-antigenic domains:

multi-antigenic domain ATP150:
[SEQ ID NO: 13]
NRTLTLFNVTRNDARAYVSGIQNSVSANRSDPVTLDVLPDSSYLSGANLN
LSCHSASPQYSWRINGIPQQHTQVLFIAKITPNNNGTYACFVSNLATGRN
NSIVKSITVSASGTSPGLSATEYKLVVVGADGVGKSALTIQLIQVAAARP
VLPAPAISDWERARRRHVGRKEGAARARVSLLPEFGTAEVHLPAPSAVRA
ARPGFGGDKGRLVGVAERQVPCRFLRPTGVLSLFLGGAVVSLQYVRPSAL
RTLLDQSAKD
multi-antigenic domain ATP152:
[SEQ ID NO: 14]
NRTLTLFNVTRNDARAYVSGIQNSVSANRSDPVTLDVLPDSSYLSGANLN
LSCHSASPQYSWRINGIPQQHTQVLFIAKITPNNNGTYACFVSNLATGRN
NSIVKSITVSASGTSPGLSATEYKLVVVGAVGVGKSALTIQLIQVAAARP
VLPAPAISDWERARRRHVGRKEGAARARVSLLPEFGTAEVHLPAPSAVRA
ARPGFGGDKGRLVGVAERQVPCRFLRPTGVLSLFLGGAVVSLQYVRPSAL
RTLLDQSAKD

As described above, the peptide constructs ATP150 and ATP152 comprise, in addition to the multi-antigenic domain, a cell penetrating peptide. The cell penetrating peptide included in ATP150 and ATP152 has the following sequence:

| [SEQ ID NO: 15] | |
|---|---|
| | KRYKNRVASRKSRAKFKQLLQHYREVAAAKSSENDRLRLLLK |
| | In addition, the peptide constructs |
| | ATP150 and ATP152 further comprise |
| | a TLR agonist: |
| [SEQ ID NO: 16] | |
| | STVHEILSKLSLEGDHSTPPSAYGSVKPYTNFDAE |

In summary, the peptide constructs ATP150 and ATP152 exhibit the following sequences:

ATP150
[SEQ ID NO: 17]
MKRYKNRVASRKSRAKFKQLLQHYREVAAAKSSENDRLRLLLKNRTLTLF
NVTRNDARAYVSGIQNSVSANRSDPVTLDVLPDSSYLSGANLNLSCHSAS
PQYSWRINGIPQQHTQVLFIAKITPNNNGTYACFVSNLATGRNNSIVKSI
TVSASGTSPGLSATEYKLVVVGADGVGKSALTIQLIQVAAARPVLPAPAI
SDWERARRRHVGRKEGAARARVSLLPEFGTAEVHLPAPSAVRAARPGFGG
DKGRLVGVAERQVPCRFLRPTGVLSLFLGGAVVSLQYVRPSALRTLLDQS
AKDSTVHEILSKLSLEGDHSTPPSAYGSVKPYTNFDAE
ATP152
[SEQ ID NO: 18]
MKRYKNRVASRKSRAKFKQLLQHYREVAAAKSSENDRLRLLLKNRTLTLF
NVTRNDARAYVSGIQNSVSANRSDPVTLDVLPDSSYLSGANLNLSCHSAS
PQYSWRINGIPQQHTQVLFIAKITPNNNGTYACFVSNLATGRNNSIVKSI
TVSASGTSPGLSATEYKLVVVGAVGVGKSALTIQLIQVAAARPVLPAPAI
SDWERARRRHVGRKEGAARARVSLLPEFGTAEVHLPAPSAVRAARPGFGG
DKGRLVGVAERQVPCRFLRPTGVLSLFLGGAVVSLQYVRPSALRTLLDQS
AKDSTVHEILSKLSLEGDHSTPPSAYGSVKPYTNFDAE

Next, the immunogenicity of the antigens/antigenic fragments contained in the multi-antigenic domain of ATP150 and ATP152 was tested in monocyte-derived human dendritic cells (moDCs). To this end, cells were extracted from fresh human Leukapheresis products named Leukopaks supplied by Tissue Solution (Scotland). The HLA typing of the 15 donors was done by the Immunology and Transplant Unit and National Reference Laboratory for Histocompatibility (LNRH) of the Geneva University Hospital. Peripheral blood mononuclear cells (PBMCs) from leukopaks samples were prepared after density gradient centrifugation on Ficoll-Paque and monocytes were enriched by cold aggregation. Remaining T lymphocytes were eliminated using rosette formation with sheep red blood cells and density gradient centrifugation on Ficoll-Paque. Monocytes were then plated with complete RPMI medium supplemented with GM-CSF and IL-4 during 7 days. Cells were then concentrated and Interferon alpha 2a was added together with 600 nM of ATP150 or of ATP152. Cells were incubated overnight at 37° C. and 5% CO₂. Cells were then scrapped, numerated. and centrifuged. The supernatant was aspirated and the cell pellet was frozen at −20° C. before ligandome analysis.

The cell pellet was lysed with a solubilization buffer and homogenized by pulsed sonification. After debris elimination and sterile filtration, HLA class I and class II molecules were isolated from the soluble fraction using standard immunoaffinity purification. The HLA ligands were then eluted by acid elution and isolated by ultracentrifugation, desalted and pre-concentrated prior to LC-MS/MS analysis.

Peptide samples were separated by nanoflow high performance liquid chromatography (RSLCnano, Thermo Fisher Scientific) using a 50 μm×25 cm PepMap rapid separation liquid chromatography column (Thermo Fisher Scientific) and a gradient ranging from 2.4% to 32.0% acetonitrile over the course of 90 min. Eluting peptides were analyzed in an online-coupled LTQ Orbitrap Fusion Lumos mass spectrometer (Thermo Fisher Scientific) using a top speed collision-induced dissociation (CID) fragmentation method for HLA class I or a higher-energy collisional dissociation (HCD) fragmentation method for HLA class II. For label-free quantification (LFQ) of the relative HLA ligand abundances between ATP150 (or ATP152)-loaded and non-loaded activated samples, the injected peptide amounts of paired samples calculated from the average precursor ion intensities determined in dose-finding runs were normalized and LC-MS/MS analysis was performed in five technical replicates for each sample.

The raw files were processed using Proteome Discoverer 1.4 (Thermo Fisher Scientific). The SEQUEST HT search engine (University of Washington) was used to search the human proteome as comprised in the Swiss-Prot database combined with the construct ATP150 (or ATP152). After data processing specific filters were applied. For class I the data were filtered with an FDR of 5%, peptide lengths of 8-12 amino acids and search engine rank 1. For class II the data were filtered with an FDR of 1%, peptide lengths of 8-25 amino acids and search engine rank 1. Protein inference was disabled, allowing for multiple protein annotations of peptides. To determine HLA class I binding peptides, the NetMHCpan-3.0 algorithm with a percentile binding rank below 2% and the SYFPEITHI algorithm with a threshold of 50% of the maximum score were used.

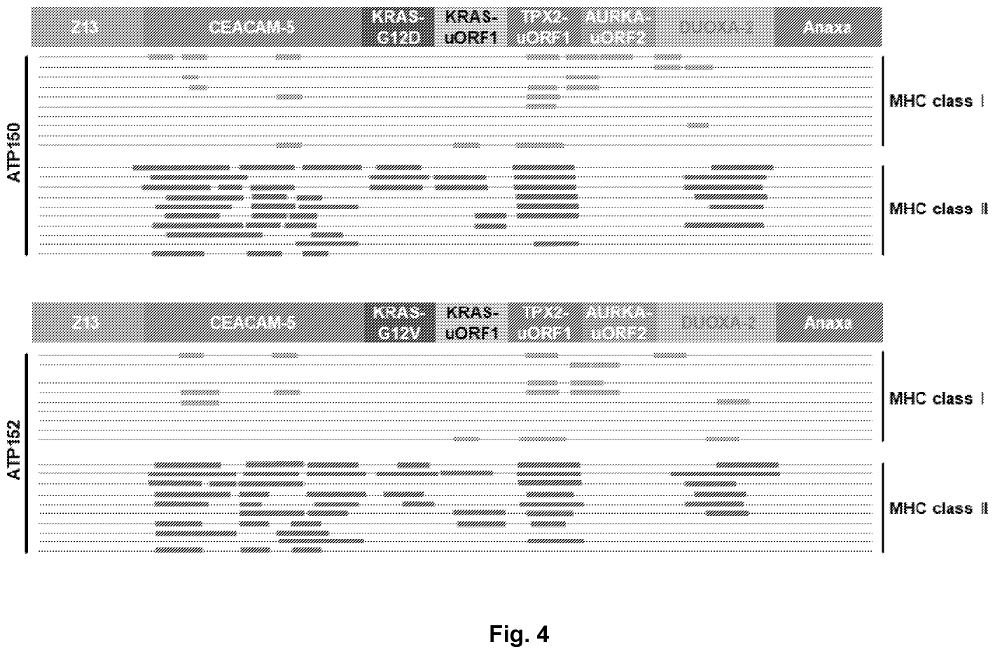

Results are shown in FIG. 4. These data show that monocyte-derived human dendritic cells (moDCs), which were incubated with ATP150 or ATP152, presented MHC class I and/or MHC class II epitopes derived from all incorporated antigens, further validating that endogenous processing in professional antigen-presenting cells can drive the display of meaningful antigens to patients' CD8 and CD4 T cells. CEACAM5 and DUOXA2 epitopes are presented by MHC class I in human GI tract tumors in vivo.

The Ligandome analysis study confirms the correct delivery of the ATP150 and ATP152 into human moDCs cells, the translation of the multi-antigenic domain of ATP150 and ATP152, and the multi-epitopic and multi-allelic processing and presentation of the all the antigens of the multi-antigenic domain of ATP150 and ATP152.

### Example 5: CEACAM5- and DUOXA2-Specific T Cell Response of ATP15/VSV-GP154 Heterologous Prime-Boost

Next, antigen-specific T-cell responses were assessed in a heterologous prime-boost vaccination scheme, wherein ATP150 (as described in Example 4 above) was combined with a recombinant vesicular stomatitis virus (VSV) encoding corresponding antigens/antigenic fragments (referred to herein as “VSV-GP154”) as described below.

The multi-antigenic domain encoded in the VSV comprises the antigens/antigenic fragments according to SEQ ID NOs 1, 3 and 5-12. The multi-antigenic domain of VSV-GP154 has the following amino acid sequence:

[SEQ ID NO: 19]
MNRTLTLFNVTRNDARAYVSGIQNSVSANRSDPVTLDVLPDSSYLSGANL
NLSCHSASPQYSWRINGIPQQHTQVLFIAKITPNNNGTYACFVSNLATGR
NNSIVKSITVSASGTSPGLSATEYKLVVVGADGVGKSALTIQLIQTEYKL
VVVGAVGVGKSALTIQLIQVAAARPVLPAPAISDWERARRRHVGRKEGAA
RARVSLLPEFGTAEVHLPAPSAVRAARPGFGGDKGRLVGVAERQVPCRFL
RPTGVLSLFLGGAVVSLQYVRPSALRTLLDQSAKD

Specifically, VSV-GP154 has a sequence according to SEQ ID NO: 30 (RNA sequence of VSV-GP154, complement without reverse). The corresponding cDNA sequence is provided in SEQ ID NO: 49.

To test antigen-specific T-cell responses were assessed in a heterologous prime-boost vaccination scheme, peripheral immune response at day 21 (1 week after VSV-GP154 boost) was investigated to determine CEACAM5 and DUOXA2-specific T cells by enzyme-linked immunospot (ELISpot) in mice. To this end, C57BL/6 mice (6 and 10 weeks old) were injected with 2 cycles of vaccination: a first s.c. vaccination with 10 nmoles of ATP150; a boost with 10 nmoles of ATP150 s.c. or 10⁷ TCID₅₀ of VSV-GP154 i.v. at day 14. At day 21, spleen were harvested. Spleen cells were isolated and assessed in ELISpot assay. ELISpot assay was performed using the Murine IFNγELISpot Diaclone kit (Ref 862.031.015S) according to manufacturer instructions. Splenocytes were isolated with Ficoll Paque Plus, GE Healthcare, ref 171440.02 and plated in a concentration gradient from 2×10⁵-1.0×10⁶ cells. Cells were pulsed with overlapping 11mer peptides corresponding to the CEACAM5 and DUOXA2 regions incorporated in the ATP150 and VSV-GP154. Spots were counted using ELISPOT reader.

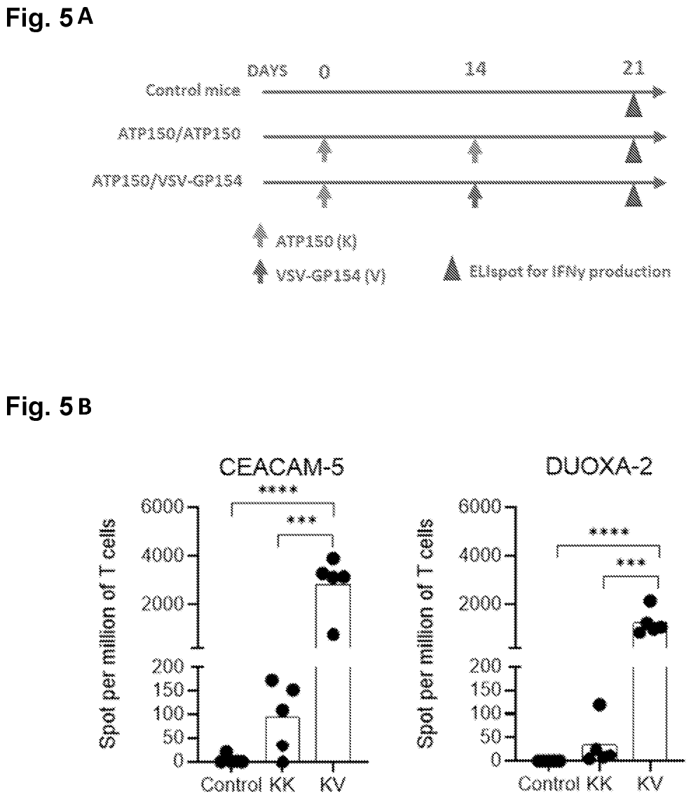

FIG. 5 shows the prime-boost protocol (A) and the results (B). Boost with VSV-GP154 promotes potent increase of CEACAM5 and DUOXA2-specific IFNγ-producing T cells as compared to homologous prime-boost.

### Example 6: Heterologous Prime-Boost Vaccination in a Mouse Tumor Model

To assess treatment effects like tumor volume and survival rates in a heterologous prime-boost vaccination regimen with a peptide construct and recombinant VSV, a mouse tumor model (TC-1 tumor model) was used. To this end, the multi-antigenic domain of the peptide construct and the multi-antigenic domain of the VSV needed to be adapted to the model tumor. The multi-antigenic domains as described in above examples 4 and 5 were designed for use in humans, while animal tumor models require vaccination with antigens/antigenic fragments corresponding to the model tumor.

To this end, the multi-antigenic domain described in above examples 4 and 5 was replaced with “multi-antigenic domain 25” (Mad-25; SEQ ID NO: 20), which contains both CD8 and CD4 H-2b epitopes from E7 HPV. Accordingly, the peptide construct of the present example comprises the multi-antigenic domain Mad25. VSV-GP-HPV encodes the attenuated E6/E7 fusion construct (Cassetti et al., 2004, Vaccine 22(3-4): 520-527) in addition to wild-type E2.

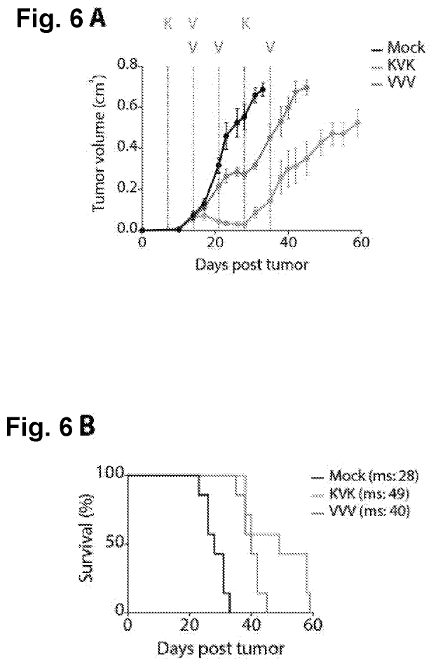

Briefly, TC-1 cells were provided by T. C. Wu (Johns Hopkins University, Maryland, US) and cultured in complete RPMI 1640 with 0.4 mg/ml geneticin. For tumor implantation, mice were injected subcutaneously with 1×10⁵ TC-1 cells in the back. Seven days later, mice were immunized with 2 nmol of the peptide construct (multi-antigenic domain “Mad25”; in FIG. 6 “K”) prime s.c. or with 1×10⁷ TCID₅₀ VSV-GP-HPV i.v. (in FIG. 6 “V”). 14 days post tumor implantation mice received 1×10⁷ TCID₅₀ VSV-GP-HPV i.v. Additional doses of the peptide construct “K” and the virus “V” were administered as indicated by dotted lines in FIG. 6A. For monitoring tumor growth, tumor diameter was measured 2-3 times per week using a caliper and volume was calculated using the formula: 0.4×length×width². Mice were sacrificed when tumor-size reached the size specified by the respective institutional veterinary authorities or tumors showed signs of ulcerations.

Results are shown in FIG. 6. Virus treatment (“V”) alone resulted in delayed tumor growth (FIG. 6A). However, virus treatment (“boost”) after the “prime” with the peptide construct led additionally to a complete remission in all tumors; even in large tumors (FIG. 6B). This strongly indicates that priming with the peptide (K) can induce tumor regression of sizeable tumors treated with virus two weeks after grafting. These data suggest that the peptide (K) prime lays the immunological foundation for the strong tumor remission that follows the virus (V) boost in the TC-1 tumor model.

### Example 7: Immunogenicity and Efficacy of Heterologous Prime-Boost in Combination with Anti-PD-1 in Tumor-Bearing Mice

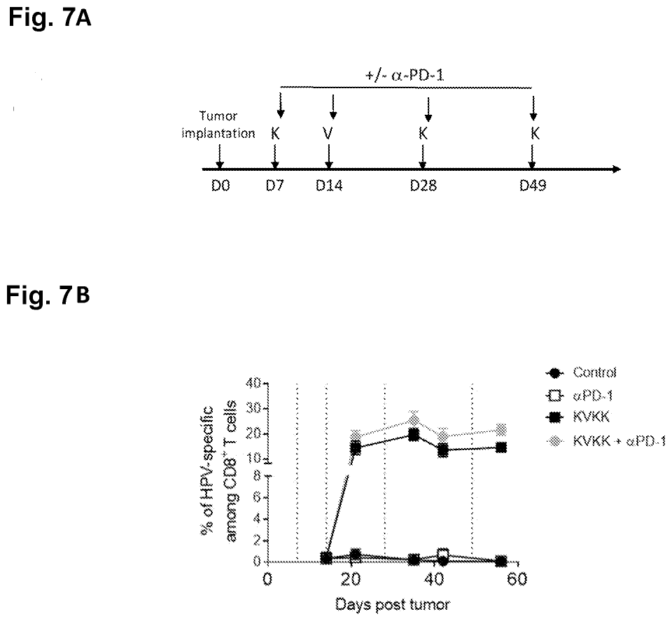

To assess immunogenicity and efficacy of combinatory treatment with heterologous prime-boost vaccination and a checkpoint inhibitor (anti-PD-1), the TC-1 mouse tumor model was used, essentially as described in Example 6 above. As described in Example 6, TC-1 cells were implanted and mice received the peptide construct “K” (with the multi-antigenic domain Mad25) and the VSV-GP-HPV “V”, as described in Example 6. Mice were also administered 200 μg i.v. of αPD-1 antibody (clone RMP1-14, BioXcell, Lebanon, New Hampshire, US) at day 7, 15, 28 and 49 according to the schedule shown in FIG. 7A. To assess circulating anti-HPV CD8 T cells, mice were bled at days 14, 21, 35, 42, 56, and multimer staining detecting HPV-specific CD8 T cells was performed and analyzed by flow cytometry.

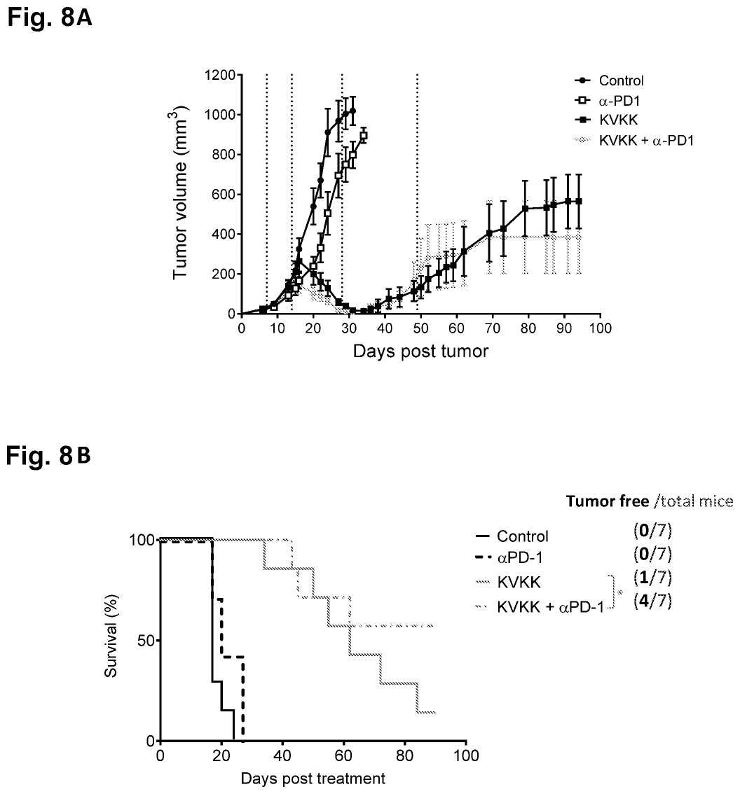

Results are shown in FIGS. 7 and 8. As shown in FIG. 7B, circulating levels of HPV-specific CD8 T cells were increased similarly in the KVKK regimen, independently whether mice received anti-PD-1 treatment or not. As shown in FIG. 8, therapeutic vaccination starting at day 7 after tumor cell implantation mediated a strong anti-tumor effect in the TC-1 tumor model in both groups treated with KVKK. Increased survival was observed when mice received additionally anti-PD-1 injection at each vaccination, with 4 mice out of 7 remaining tumor-free in the triple combination group (FIG. 8B).

### Example 8: Effects of Different VSV Doses in Heterologous Prime-Boost Vaccination

To assess the efficacy of different doses of VSV-GP-HPV “V” in heterologous prime-boost vaccination, the TC-1 tumor model, as described in examples 6 and 7 above, was used. For tumor implantation, mice were injected subcutaneously with 1×10⁵ TC-1 cells in the back. Seven days later, mice were immunized with 2 nmol of the peptide construct (“K”) prime s.c. VSV-GP-HPV boost was administrated at 3 different doses: 1×10⁷, 3×10⁷ or 5×10⁷ TCID₅₀ at day 14 after tumor cell implantation. Fourteen days later, mice received 2 nmol of peptide construct (“K”).

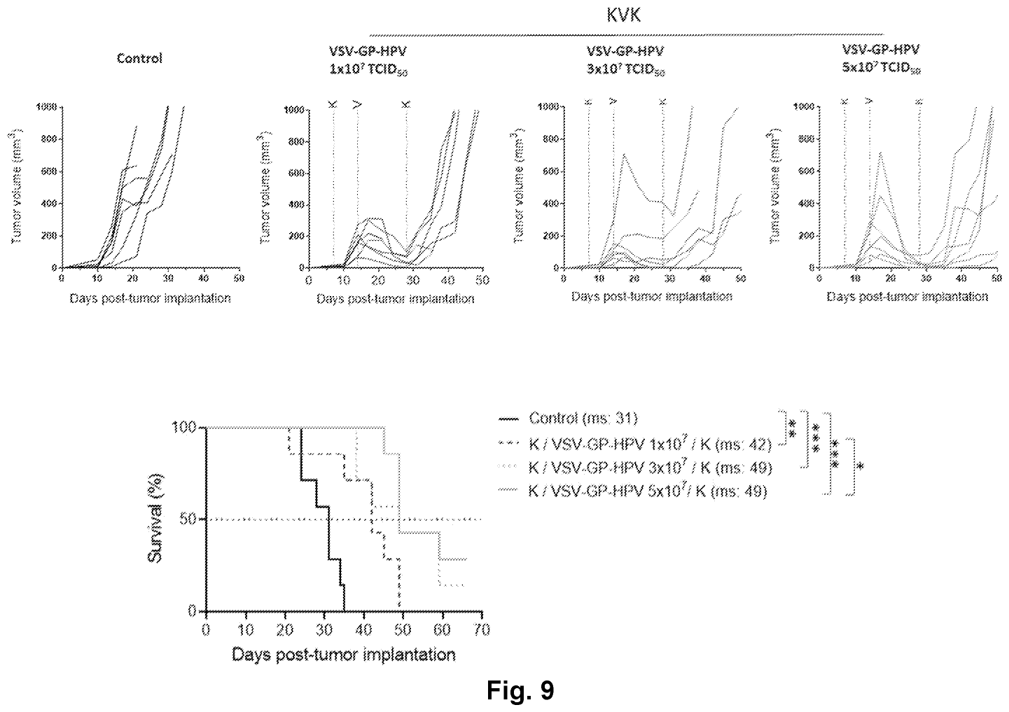

Results are shown in FIG. 9. The anti-tumor effect observed previously is highlighted by a similar transient remission in the three K/V/K groups as observed by the strong decreased of the tumor volume after the first boost (VSV-GP-HPV injection). Although the group at VSV-GP-HPV 5×10⁷ TCID₅₀ shows a slightly enhanced survival, no major differences are observed in the 3 treated groups displaying the different doses of VSV-GP-HPV.

### Example 9: Effects of Plurality of Immunogenic Epitopes Including the Fragments of KRAS-uORF1. TPX2-uORF1 and AURKA-uORF2

To investigate the effects of a plurality of immunogenic epitopes including the fragments of KRAS-uORF1, TPX2-uORF1 and AURKA-uORF2 in the peptide according to the present invention (ATP150), a comparative construct lacking the fragments of KRAS-uORF1, TPX2-uORF1 and AURKA-uORF2 (“ATP132”) was prepared.

The multi-antigenic domain of ATP132 has to following sequence:

[SEQ ID NO: 50]
NRTLTLFNVTRNDARAYVSGIQNSVSANRSDPVTLDVLPDSSYLSGANLN
LSCHSASPQYSWRINGIPQQHTQVLFIAKITPNNNGTYACFVSNLATGRN
NSIVKSITVSASGTSPGLSATEYKLVVVGADGVGKSALTIQLIQ

In addition to said multi-antigenic domain, ATP132 comprises the cell penetrating peptide according to SEQ ID NO: 15 and the TLR agonist according to SEQ ID NO: 16.

In summary, the peptide construct ATP132 exhibits the following sequence:

[SEQ ID NO: 51]
MKRYKNRVASRKSRAKFKQLLQHYREVAAAKSSENDRLRLLLKNRTLTLF
NVTRNDARAYVSGIQNSVSANRSDPVTLDVLPDSSYLSGANLNLSCHSAS
PQYSWRINGIPQQHTQVLFIAKITPNNNGTYACFVSNLATGRNNSIVKSI
TVSASGTSPGLSATEYKLVVVGADGVGKSALTIQLIQSTVHEILSKLSLE
GDHSTPPSAYGSVKPYTNFDAE

The immunogenicity of the antigens/antigenic fragments contained in the multi-antigenic domain of ATP150 and ATP132 was compared in monocyte-derived human dendritic cells (moDCs). To this end, cells were extracted from fresh human Leukapheresis products named Leukopaks supplied by Tissue Solution (Scotland). The HLA typing of the donors was done by the Immunology and Transplant Unit and National Reference Laboratory for Histocompatibility (LNRH) of the Geneva University Hospital. Peripheral blood mononuclear cells (PBMCs) from leukopaks samples were prepared after density gradient centrifugation on Ficoll-Paque and monocytes were enriched by cold aggregation. Remaining T lymphocytes were eliminated using rosette formation with sheep red blood cells and density gradient centrifugation on Ficoll-Paque. Monocytes were then plated with complete RPMI medium supplemented with GM-CSF and IL-4 during 7 days. Cells were then concentrated and Interferon alpha 2a was added together with 600 nM of ATP150 or 600 nM of ATP132. Cells were incubated overnight at 37° C. and 5% CO₂. Cells were then scrapped, numerated. and centrifuged. The supernatant was aspirated, and the cell pellet was frozen at −20° C. before ligandome analysis.

The cell pellet was lysed with a solubilization buffer and homogenized by pulsed sonification. After debris elimination and sterile filtration, HLA class I and class II molecules were isolated from the soluble fraction using standard immunoaffinity purification. The HLA ligands were then eluted by acid elution and isolated by ultracentrifugation, desalted and pre-concentrated prior to LC-MS/MS analysis.

Peptide samples were separated by nanoflow high performance liquid chromatography (RSLCnano, Thermo Fisher Scientific) using a 50 μm×25 cm PepMap rapid separation liquid chromatography column (Thermo Fisher Scientific) and a gradient ranging from 2.4% to 32.0% acetonitrile over the course of 90 min. Eluting peptides were analyzed in an online-coupled LTQ Orbitrap Fusion Lumos mass spectrometer (Thermo Fisher Scientific) using a top speed collision-induced dissociation (CID) fragmentation method for HLA class I or a higher-energy collisional dissociation (HCD) fragmentation method for HLA class 11. For label-free quantification (LFQ) of the relative HLA ligand abundances between ATP150- or ATP132-loaded and non-loaded activated samples, the injected peptide amounts of paired samples calculated from the average precursor ion intensities determined in dose-finding runs were normalized and LC-MS/MS analysis was performed in five technical replicates for each sample.

The raw files were processed using Proteome Discoverer 1.4 (Thermo Fisher Scientific). The SEQUEST HT search engine (University of Washington) was used to search the human proteome as comprised in the Swiss-Prot database combined with the construct ATP150 or ATP132. After data processing specific filters were applied. For class I the data were filtered with an FDR of 5%, peptide lengths of 8-12 amino acids and search engine rank 1. For class II the data were filtered with an FDR of 1%, peptide lengths of 8-25 amino acids and search engine rank 1. Protein inference was disabled, allowing for multiple protein annotations of peptides. To determine HLA class I binding peptides, the NetMHCpan-3.0 algorithm with a percentile binding rank below 2% and the SYFPEITHI algorithm with a threshold of 50% of the maximum score were used.

The results are depicted in Table 2 below:

TABLE 2

| # peptide/donor | ATP132 | ATP150 |
|---|---|---|
| MHC I | 1.2 | 2.0 |
| MHC II | 22.8 | 79.3 |

In Table 2, the average number of detected peptide epitopes for MHC class I and MHC class II per donor is shown.

These data show that monocyte-derived human dendritic cells (moDCs), which were incubated with a peptide according to the present invention comprising fragments of KRAS-uORF1, TPX2-uORF1 and AURKA-uORF2 (ATP150) presented more MHC class I and/or MHC class II epitopes, which are derived from all incorporated antigens, than moDCs incubated with the comparative peptide lacking the fragments of KRAS-uORF1, TPX2-uORF1 and AURKA-uORF2 (ATP132). These data further validate that a higher number of immunogenic fragments result in a superior endogenous processing in professional antigen-presenting cells which can drive the display of meaningful antigens to patients' CD8 and CD4 T cells.

TABLE OF SEQUENCES AND SEQ ID NUMBERS (SEQUENCE LISTING):

| SEQ ID NO | Sequence | Remarks |
|---|---|---|
| SEQ ID NO: 1 | VAAARPVLPAPAISDWERARRRH | KRAS-uORF1 |
| | | (short) |
| SEQ ID NO: 2 | VAAAKVAAARPVLPAPAISDWERARRRH | KRAS-uORF1 |
| | | (long) |
| SEQ ID NO: 3 | VGRKEGAARARVSLLPEFGTAEVHLPAPSAVRAARPGF | TPX2-uORF1 |
| | | (long) |
| SEQ ID NO: 4 | TAEVHLPAPSAVRAARPGF | TPX2-uORF1 |
| | | (short) |
| SEQ ID NO: 5 | GGDKGRLVGVAERQVPCRFLRP | AURKA-uORF2 |
| SEQ ID NO: 6 | NRTLTLFNVTRNDARAYVSGIQNSVSANRSDPVTLDVL | CEACAM5 |
| | | fragment |
| SEQ ID NO: 7 | PDSSYLSGANLNLSCHSAS | CEACAM5 |
| | | fragment |
| SEQ ID NO: 8 | PQYSWRINGIPQQHTQVLFIAKITPNNNGTYACFVSNLA | CEACAM5 |
| | TGRNNSIVKSITVSASGTSPGLSA | fragment |
| SEQ ID NO: 9 | NRTLTLFNVTRNDARAYVSGIQNSVSANRSDPVTLDVLP | CEACAM5 |
| | DSSYLSGANLNLSCHSASPQYSWRINGIPQQHTQVLFIA | fragment |
| | KITPNNNGTYACFVSNLATGRNNSIVKSITVSASGTSPG | fusion |
| | LSA | construct |
| SEQ ID NO: 10 | TGVLSLFLGGAVVSLQYVRPSALRTLLDQSAKD | DUOXA2 |
| | | fragment |
| SEQ ID NO: 11 | TEYKLVVVGADGVGKSALTIQLIQ | KRAS-G12D |
| | | fragment |
| SEQ ID NO: 12 | TEYKLVVVGAVGVGKSALTIQLIQ | KRAS-G12V |
| | | fragment |
| SEQ ID NO: 13 | NRTLTLFNVTRNDARAYVSGIQNSVSANRSDPVTLDVLP | multi-antigenic |
| | DSSYLSGANLNLSCHSASPQYSWRINGIPQQHTQVLFIA | domain ATP150 |
| | KITPNNNGTYACFVSNLATGRNNSIVKSITVSASGTSPG | |
| | LSATEYKLVVVGADGVGKSALTIQLIQVAAARPVLPAPA | |
| | ISDWERARRRHVGRKEGAARARVSLLPEFGTAEVHLPAP | |
| | SAVRAARPGFGGDKGRLVGVAERQVPCRFLRPTGVLSLF | |
| | LGGAVVSLQYVRPSALRTLLDQSAKD | |
| SEQ ID NO: 14 | NRTLTLFNVTRNDARAYVSGIQNSVSANRSDPVTLDVLP | multi-antigenic |
| | DSSYLSGANLNLSCHSASPQYSWRINGIPQQHTQVLFIA | domain ATP152 |
| | KITPNNNGTYACFVSNLATGRNNSIVKSITVSASGTSPG | |
| | LSATEYKLVVVGAVGVGKSALTIQLIQVAAARPVLPAPA | |
| | ISDWERARRRHVGRKEGAARARVSLLPEFGTAEVHLPAP | |
| | SAVRAARPGFGGDKGRLVGVAERQVPCRFLRPTGVLSLF | |
| | LGGAVVSLQYVRPSALRTLLDQSAKD | |
| SEQ ID NO: 15 | KRYKNRVASRKSRAKFKQLLQHYREVAAAKSSENDRLRL | cell penetrating |
| | LLK | peptide (Z13) |
| SEQ ID NO: 16 | STVHEILSKLSLEGDHSTPPSAYGSVKPYTNFDAE | TLR agonist |
| | | (Anaxa) |
| SEQ ID NO: 17 | MKRYKNRVASRKSRAKFKQLLQHYREVAAAKSSENDRLR | ATP150 |
| | LLLKNRTLTLFNVTRNDARAYVSGIQNSVSANRSDPVTL | |
| | DVLPDSSYLSGANLNLSCHSASPQYSWRINGIPQQHTQV | |
| | LFIAKITPNNNGTYACFVSNLATGRNNSIVKSITVSASG | |
| | TSPGLSATEYKLVVVGADGVGKSALTIQLIQVAAARPVL | |
| | PAPAISDWERARRRHVGRKEGAARARVSLLPEFGTAEVH | |
| | LPAPSAVRAARPGFGGDKGRLVGVAERQVPCRFLRPTGV | |
| | LSLFLGGAVVSLQYVRPSALRTLLDQSAKDSTVHEILSK | |
| | LSLEGDHSTPPSAYGSVKPYTNFDAE | |
| SEQ ID NO: 18 | MKRYKNRVASRKSRAKFKQLLQHYREVAAAKSSENDRLR | ATP152 |
| | LLLKNRTLTLFNVTRNDARAYVSGIQNSVSANRSDPVTL | |
| | DVLPDSSYLSGANLNLSCHSASPQYSWRINGIPQQHTQV | |
| | LFIAKITPNNNGTYACFVSNLATGRNNSIVKSITVSASG | |
| | TSPGLSATEYKLVVVGAVGVGKSALTIQLIQVAAARPVL | |
| | PAPAISDWERARRRHVGRKEGAARARVSLLPEFGTAEVH | |
| | LPAPSAVRAARPGFGGDKGRLVGVAERQVPCRFLRPTGV | |
| | LSLFLGGAVVSLQYVRPSALRTLLDQSAKDSTVHEILSK | |
| | LSLEGDHSTPPSAYGSVKPYTNFDAE | |
| SEQ ID NO: 19 | MNRTLTLFNVTRNDARAYVSGIQNSVSANRSDPVTLDVL | multi-antigenic |
| | PDSSYLSGANLNLSCHSASPQYSWRINGIPQQHTQVLFI | domain VSV-GP154 |
| | AKITPNNNGTYACFVSNLATGRNNSIVKSITVSASGTSP | |
| | GLSATEYKLVVVGADGVGKSALTIQLIQTEYKLVVVGAV | |
| | GVGKSALTIQLIQVAAARPVLPAPAISDWERARRRHVGR | |
| | KEGAARARVSLLPEFGTAEVHLPAPSAVRAARPGFGGDK | |
| | GRLVGVAERQVPCRFLRPTGVLSLFLGGAVVSLQYVRPS | |
| | ALRTLLDQSAKD | |
| SEQ ID NO: 20 | QAEPDRAHYNIVTFSSKS | multi-antigenic |
| | | domain Mad25 |
| SEQ ID NO: 21 | KRYKNRVASRKSRAKFKQLLQHYREVAAAK | cell penetrating |
| | | peptide (Z14) |
| SEQ ID NO: 22 | KRYKNRVASRKSRAKFK | cell penetrating |
| | | peptide (Z15) |
| SEQ ID NO: 23 | REVAAAKSSENDRLRLLLK | cell penetrating |
| | | peptide (Z18) |
| SEQ ID NO: 24 | STVHEILCKLSLEGDHSTPPSAYGSVKPYTNFDAE | TLR agonist |
| | | (Anaxa variant) |
| SEQ ID NO: 25 | MGQIVTMFEALPHIIDEVINIVIIVLIIITSIKAVYNFA | GP of LCMV |
| | TCGILALVSFLFLAGRSCGMYGLNGPDIYKGVYQFKSVE | |
| | FDMSHLNLTMPNACSANNSHHYISMGSSGLELTFTNDSI | |
| | LNHNFCNLTSAFNKKTFDHTLMSIVSSLHLSIRGNSNHK | |
| | AVSCDFNNGITIQYNLSFSDPQSAISQCRTFRGRVLDMF | |
| | RTAFGGKYMRSGWGWAGSDGKTTWCSQTSYQYLIIQNRT | |
| | WENHCRYAGPFGMSRILFAQEKTKFLTRRLAGTFTWTLS | |
| | DSSGVENPGGYCLTKWMILAAELKCFGNTAVAKCNVNHD | |
| | EEFCDMLRLIDYNKAALSKFKQDVESALHVFKTTVNSLI | |
| | SDQLLMRNHLRDLMGVPYCNYSKFWYLEHAKTGETSVPK | |
| | CWLVTNGSYLNETHFSDQIEQEADNMITEMLRKDYIKRQ | |
| | GSTPLALMDLLMFSTSAYLISIFLHLVKIPTHRHIKGGS | |
| | CPKPHRLTNKGICSCGAFKVPGVKTIWKRR | |
| SEQ ID NO: 26 | MSVTVKRIIDNTVVVPKLPANEDPVEYPADYFRKSKEIP | vesicular |
| | LYINTTKSLSDLRGYVYQGLKSGNVSIIHVNSYLYGALK | stomatitis |
| | DIRGKLDKDWSSFGINIGKAGDTIGIFDLVSLKALDGVL | virus |
| | PDGVSDASRTSADDKWLPLYLLGLYRVGRTQMPEYRKKL | nucleoprotein |
| | MDGLTNQCKMINEQFEPLVPEGRDIFDVWGNDSNYTKIV | (N) |
| | AAVDMFFHMFKKHECASFRYGTIVSRFKDCAALATFGHL | |
| | CKITGMSTEDVTTWILNREVADEMVQMMLPGQEIDKADS | |
| | YMPYLIDFGLSSKSPYSSVKNPAFHFWGQLTALLLRSTR | |
| | ARNARQPDDIEYTSLTTAGLLYAYAVGSSADLAQQFCVG | |
| | DNKYTPDDSTGGLTTNAPPQGRDVVEWLGWFEDQNRKPT | |
| | PDMMQYAKRAVMSLQGLREKTIGKYAKSEFDK | |
| SEQ ID NO: 27 | MDNLTKVREYLKSYSRLDQAVGEIDEIEAQRAEKSNYEL | vesicular |
| | FQEDGVEEHTKPSYFQAADDSDTESEPEIEDNQGLYAPD | stomatitis |
| | PEAEQVEGFIQGPLDDYADEEVDVVFTSDWKQPELESDE | virus |
| | HGKTLRLTSPEGLSGEQKSQWLSTIKAVVQSAKYWNLAE | phosphoprotein |
| | CTFEASGEGVIMKERQITPDVYKVTPVMNTHPSQSEAVS | (P) |
| | DVWSLSKTSMTFQPKKASLQPLTISLDELFSSRGEFISV | |
| | GGDGRMSHKEAILLGLRYKKLYNQARVKYSL | |
| SEQ ID NO: 28 | MEVHDFETDEFNDFNEDDYATREFLNPDERMTYLNHADY | vesicular |
| | NLNSPLISDDIDNLIRKENSLPIPSMWDSKNWDGVLEML | stomatitis |
| | TSCQANPIPTSQMHKWMGSWLMSDNHDASQGYSFLHEVD | virus large |
| | KEAEITFDVVETFIRGWGNKPIEYIKKERWTDSFKILAY | protein |
| | LCQKFLDLHKLTLILNAVSEVELLNLARTFKGKVRRSSH | (L) |
| | GTNICRIRVPSLGPTFISEGWAYFKKLDILMDRNFLLMV | |
| | KDVIIGRMQTVLSMVCRIDNLFSEQDIFSLLNIYRIGDK | |
| | IVERQGNFSYDLIKMVEPICNLKLMKLARESRPLVPQFP | |
| | HFENHIKTSVDEGAKIDRGIRFLHDQIMSVKTVDLTLVI | |
| | YGSFRHWGHPFIDYYTGLEKLHSQVTMKKDIDVSYAKAL | |
| | ASDLARIVLFQQFNDHKKWFVNGDLLPHDHPFKSHVKEN | |
| | TWPTAAQVQDFGDKWHELPLIKCFEIPDLLDPSIIYSDK | |
| | SHSMNRSEVLKHVRMNPNTPIPSKKVLQTMLDTKATNWK | |
| | EFLKEIDEKGLDDDDLIIGLKGKERELKLAGRFFSLMSW | |
| | KLREYFVITEYLIKTHFVPMFKGLTMADDLTAVIKKMLD | |
| | SSSGQGLKSYEAICIANHIDYEKWNNHQRKLSNGPVFRV | |
| | MGQFLGYPSLIERTHEFFEKSLIYYNGRPDLMRVHNNTL | |
| | INSTSQRVCWQGQEGGLEGLRQKGWSILNLLVIQREAKI | |
| | RNTAVKVLAQGDNQVICTQYKTKKSRNVVELQGALNQMV | |
| | SNNEKIMTAIKIGTGKLGLLINDDETMQSADYLNYGKIP | |
| | IFRGVIRGLETKRWSRVTCVTNDQIPTCANIMSSVSTNA | |
| | LTVAHFAENPINAMIQYNYFGTFARLLLMMHDPALRQSL | |
| | YEVQDKIPGLHSSTFKYAMLYLDPSIGGVSGMSLSRFLI | |
| | RAFPDPVTESLSFWRFIHVHARSEHLKEMSAVFGNPEIA | |
| | KFRITHIDKLVEDPTSLNIAMGMSPANLLKTEVKKCLIE | |
| | SRQTIRNQVIKDATIYLYHEEDRLRSFLWSINPLFPRFL | |
| | SEFKSGTFLGVADGLISLFQNSRTIRNSFKKKYHRELDD | |
| | LIVRSEVSSLTHLGKLHLRRGSCKMWTCSATHADTLRYK | |
| | SWGRTVIGTTVPHPLEMLGPQHRKETPCAPCNTSGFNYV | |
| | SVHCPDGIHDVFSSRGPLPAYLGSKTSESTSILQPWERE | |
| | SKVPLIKRATRLRDAISWFVEPDSKLAMTILSNIHSLTG | |
| | EEWTKRQHGFKRTGSALHRFSTSRMSHGGFASQSTAALT | |
| | RLMATTDTMRDLGDQNFDFLFQATLLYAQITTTVARDGW | |
| | ITSCTDHYHIACKSCLRPIEEITLDSSMDYTPPDVSHVL | |
| | KTWRNGEGSWGQEIKQIYPLEGNWKNLAPAEQSYQVGRC | |
| | IGFLYGDLAYRKSTHAEDSSLFPLSIQGRIRGRGFLKGL | |
| | LDGLMRASCCQVIHRRSLAHLKRPANAVYGGLIYLIDKL | |
| | SVSPPFLSLTRSGPIRDELETIPHKIPTSYPTSNRDMGV | |
| | IVRNYFKYQCRLIEKGKYRSHYSQLWLFSDVLSIDFIGP | |
| | FSISTTLLQILYKPFLSGKDKNELRELANLSSLLRSGEG | |
| | WEDIHVKFFTKDILLCPEEIRHACKFGIAKDNNKDMSYP | |
| | PWGRESRGTITTIPVYYTTTPYPKMLEMPPRIQNPLLSG | |
| | IRLGQLPTGAHYKIRSILHGMGIHYRDFLSCGDGSGGMT | |
| | AALLRENVHSRGIFNSLLELSGSVMRGASPEPPSALETL | |
| | GGDKSRCVNGETCWEYPSDLCDPRTWDYFLRLKAGLGLQ | |
| | IDLIVMDMEVRDSSTSLKIETNVRNYVHRILDEQGVLIY | |
| | KTYGTYICESEKNAVTILGPMFKTVDLVQTEFSSSQTSE | |
| | VYMVCKGLKKLIDEPNPDWSSINESWKNLYAFQSSEQEF | |
| | ARAKKVSTYFTLTGIPSQFIPDPFVNIETMLQIFGVPTG | |
| | VSHAAALKSSDRPADLLTISLFYMAIISYYNINHIRVGP | |
| | IPPNPPSDGIAQNVGIAITGISFWLSLMEKDIPLYQQCL | |
| | AVIQQSFPIRWEAVSVKGGYKQKWSTRGDGLPKDTRISD | |
| | SLAPIGNWIRSLELVRNQVRLNPFNEILFNQLCRTVDNH | |
| | LKWSNLRRNTGMIEWINRRISKEDRSILMLKSDLHEENS | |
| | WRD | |
| SEQ ID NO: 29 | MSSLKKILGLKGKGKKSKKLGIAPPPYEEDTSMEYAPSA | vesicular |
| | PIDKSYFGVDEMDTYDPNQLRYEKFFFTVKMTVRSNRPF | stomatitis |
| | RTYSDVAAAVSHWDHMYIGMAGKRPFYKILAFLGSSNLK | virus matrix |
| | ATPAVLADQGQPEYHAHCEGRAYLPHRMGKTPPMLNVPE | protein |
| | HFRRPFNIGLYKGTIELTMTIYDDESLEAAPMIWDHFNS | (M) |
| | SKFSDFREKALMFGLIVEKKASGAWVLDSIGHFK | |
| SEQ ID NO: 30 | UGCUUCUGUUUGUUUGGUAAUAAUAGUAAUUUUCCGAGU | RNA sequence |
| | CCUCUUUGAAAUUGUCAUUAGUUUUACAGACAAUGUCAG | VSV-GP154 |
| | UUCUCUUAGUAACUGUUGUGUCAGCAUCAAGGUUUUGAA | (complement |
| | GGACGUUUACUCCUAGGUCACCUUAUGGGCCGUCUAAUG | without |
| | AAGUCUUUUAGUUUCCUCUAAGGAGAAAUGUAGUUAUGA | reverse) |
| | UGUUUUUCAAACAGUCUAGAUUCUCCUAUACAGAUGGUU | |
| | CCGGAGUUUAGGCCUUUACAUAGUUAGUAUGUACAGUUG | |
| | UCGAUGAACAUACCUCGUAAUUUCCUGUAGGCCCCAUUC | |
| | AACCUAUUUCUAACCAGUUCAAAGCCUUAUUUGUAGCCC | |
| | UUUCGUCCCCUAUGUUAGCCUUAUAAACUGGAACAUAGG | |
| | AACUUUCGGGACCUGCCGCAUGAAGGUCUACCUCAUAGC | |
| | CUACGAAGGUCUUGGUCGCGUCUACUGUUUACCAACGGA | |
| | AACAUAGAUGAACCGAAUAUGUCUCACCCGUCUUGUGUU | |
| | UACGGACUUAUGUCUUUUUUCGAGUACCUACCCGACUGU | |
| | UUAGUUACGUUUUACUAGUUACUUGUCAAACUUGGAGAA | |
| | CACGGUCUUCCAGCACUGUAAAAACUACACACCCCUUUA | |
| | CUGUCAUUAAUGUGUUUUUAACAGCGACGUCACCUGUAC | |
| | AAGAAGGUGUACAAGUUUUUUGUACUUACACGGAGCAAG | |
| | UCUAUGCCUUGAUAACAAAGGUCUAAGUUUCUAACACGA | |
| | CGUAACCGUUGUAAACCUGUGGAGACGUUUUAUUGGCCU | |
| | UACAGAUGUCUUCUACAUUGCUGGACCUAGAACUUGGCU | |
| | CUUCAACGUCUACUUUACCAGGUUUACUACGAAGGUCCG | |
| | GUUCUUUAACUGUUCCGGCUAAGUAUGUACGGAAUAAAC | |
| | UAGCUGAAACCUAACAGAAGAUUCAGAGGUAUAAGAAGG | |
| | CAGUUUUUGGGACGGAAGGUGAAGACCCCCGUUAACUGU | |
| | CGAGAAGACGAGUCUAGGUGGUCUCGUUCCUUACGGGCU | |
| | GUCGGACUACUGUAACUCAUAUGUAGAGAAUGAUGUCGU | |
| | CCAAACAACAUGCGAAUACGUCAUCCUAGGAGACGGCUG | |
| | AACCGUGUUGUCAAAACACAACCUCUAUUGUUUAUGUGA | |
| | GGUCUACUAUCAUGGCCUCCUAACUGCUGAUUACGUGGC | |
| | GGUGUUCCGUCUCUACACCAGCUUACCGAGCCUACCAAA | |
| | CUUCUAGUUUUGUCUUUUGGCUGAGGACUAUACUACGUC | |
| | AUACGCUUUUCUCGUCAGUACAGUGACGUUCCGGAUUCU | |
| | CUCUUCUGUUAACCGUUCAUACGAUUCAGUCUUAAACUG | |
| | UUUACUGGGAUAUUAAGAGUCUAGUGGAUAAUAUAUAAU | |
| | ACGAUGUAUACUUUUUUUGAUUGUCUAUAGUACCUAUUA | |
| | GAGUGUUUUCAAGCACUCAUAGAGUUCAGGAUAAGAGCA | |
| | GACCUAGUCCGCCAUCCUCUCUAUCUACUCUAGCUUCGU | |
| | GUUGCUCGACUUUUCAGGUUAAUACUCAACAAGGUUCUC | |
| | CUACCUCACCUUCUCGUAUGAUUCGGGAGAAUAAAAGUC | |
| | CGUCGUCUACUAAGACUGUGUCUUAGACUUGGUCUUUAA | |
| | CUUCUGUUAGUUCCGAACAUACGUGGUCUAGGUCUUCGA | |
| | CUCGUUCAACUUCCGAAAUAUGUCCCCGGAAAUCUACUG | |
| | AUACGUCUACUCCUUCACCUACAACAUAAAUGAAGCCUG | |
| | ACCUUUGUCGGACUCGAACUUAGACUGCUCGUACCUUUC | |
| | UGGAAUGCCAACUGUAGCGGUCUCCCAAAUUCACCUCUC | |
| | GUCUUUAGGGUCACCGAAAGCUGCUAAUUUCGUCAGCAC | |
| | GUUUCACGGUUUAUGACCUUAGACCGUCUCACGUGUAAA | |
| | CUUCGUAGCCCUCUUCCCCAGUAAUACUUCCUCGCGGUC | |
| | UAUUGAGGCCUACAUAUAUUCCAGUGAGGUCACUACUUG | |
| | UGUGUAGGCAGGGUUAGUCUUCGUCAUAGUCUACAAACC | |
| | AGAGAGAGUUUCUGUAGGUACUGAAAGGUUGGGUUCUUU | |
| | CGUUCAGAAGUCGGAGAGUGGUAUAGGAACCUACUUAAC | |
| | AAGAGUAGAUCUCCUCUCAAGUAGAGACAGCCUCCACUG | |
| | CCUGCUUACAGAGUAUUUCUCCGGUAGGACGAGCCGGAC | |
| | UCUAUGUUUUUCAACAUGUUAGUCCGCUCUCAGUUUAUA | |
| | AGAGACAUCUGAUACUUUUUUUCAUUGUCUAUAGUGCUA | |
| | GAUUCACAAUAGGGUUAGGUAAGUAGUACUCAAGGAAUU | |
| | UCUUCUAAGAGCCAGACUUCCCCUUUCCAUUCUUUAGAU | |
| | UCUUUAAUCCCUAGCGUGGUGGGGGAAUACUUCUCCUGU | |
| | GAUCGUACCUCAUACGAGGCUCGCGAGGUUAACUGUUUA | |
| | GGAUAAAACCUCAACUGCUCUACCUGUGGAUACUAGGCU | |
| | UAGUUAAUUCUAUACUCUUUAAGAAGAAAUGUCACUUUU | |
| | ACUGCCAAUCUAGAUUAGCAGGCAAGUCUUGUAUGAGUC | |
| | UACACCGUCGGCGACAUAGGGUAACCCUAGUGUACAUGU | |
| | AGCCUUACCGUCCCUUUGCAGGGAAGAUGUUUUAGAACC | |
| | GAAAAAACCCAAGAAGAUUAGAUUUCCGGUGAGGUCGCC | |
| | AUAACCGUCUAGUUCCAGUUGGUCUCAUAGUGCGAGUGA | |
| | CGCUUCCGUCCCGAAUAAACGGUGUAUCCUACCCCUUCU | |
| | GGGGAGGGUACGAGUUACAUGGUCUCGUGAAGUCUUCUG | |
| | GUAAGUUAUAUCCAGAAAUGUUCCCUUGCUAACUCGAGU | |
| | GUUACUGGUAGAUGCUACUACUCAGUGACCUUCGUCGAG | |
| | GAUACUAGACCCUAGUAAAGUUAAGAAGGUUUAAAAGAC | |
| | UAAAGUCUCUCUUCCGGAAUUACAAACCGGACUAACAGC | |
| | UCUUUUUCCGUAGACCUCGCACCCAGGACCUGAGAUAGC | |
| | CGGUGAAGUUUACUCGAUCAGAUUGAAGAUCGAAGACUU | |
| | GUUAGGGGCCAAAUGAGUCAGAGGGGAUUAAGGUCGGAG | |
| | AGCUUGUUGAUUAUAGGACAGAAAAGAUAGGGAUACUUU | |
| | UUUUGAUUGUCUCUAGCUAGACAAAUGCGCAGUGCCUAG | |
| | GGGGCCCGACGUCCUUAAGCGGUGGUACCCGGUCUAGCA | |
| | CUGGUACAAGCUCCGGGACGGGGUGUAGUAGCUGCUCCA | |
| | CUAGUUGUAGCACUAGUAGCACGAGUAGUAGUAGUGGUC | |
| | GUAGUUCCGGCACAUGUUGAAGCGGUGGACGCCGUAGGA | |
| | CCGGGACCACUCGAAGGACAAGGACCGGCCGUCUUCGAC | |
| | GCCGUACAUGCCGGACUUACCGGGGCUAUAGAUGUUCCC | |
| | GCACAUGGUCAAGUUCUCGCACCUCAAGCUGUACUCGGU | |
| | GGACUUGGAGUGGUACGGGUUGCGGACGUCGCGGUUGUU | |
| | AUCGGUGGUGAUGUAGUCGUACCCGUCGUCGCCGGACCU | |
| | CAACUGGAAGUGGUUGCUGUCGUAGGACUUGGUGUUGAA | |
| | GACGUUGGAGUGGUCGCGGAAGUUGUUCUUUUGGAAGCU | |
| | GGUGUGGGAGUACUCGUAGCACUCGUCGGACGUGGACUC | |
| | GUAGUCUCCGUUGUCGUUGGUGUUCCGGCACUCGACGCU | |
| | GAAGUUGUUGCCGUAGUGGUAGGUCAUGUUGGACUCGAA | |
| | GUCGCUAGGAGUCUCGCGGUAGUCGGUCACGUCUUGGAA | |
| | GUCUCCGUCUCACGACCUGUACAAGUCUUGGCGGAAGCC | |
| | GCCGUUCAUGUACUCUUCGCCGACCCCGACCCGGCCGUC | |
| | GCUGCCGUUCUGGUGGACCACGUCGGUCUGGUCGAUGGU | |
| | CAUGGAGUAGUAGGUCUUGUCUUGGACCCUCUUGGUGAC | |
| | GUCUAUGCGGCCUGGAAAGCCGUACUCGUCUUAGGACAA | |
| | GCGGGUCCUCUUUUGGUUCAAGGAGUGGUCCUCUGACCG | |
| | GCCGUGGAAGUGGACCUGGGACUCGCUGUCGUCGCCGCA | |
| | CCUCUUGGGACCGCCGAUGACGGAGUGGUUCACCUACUA | |
| | GGACCGGCGGCUCGACUUCACGAAGCCGUUGUGGCGGCA | |
| | CCGGUUCACGUUGCACUUGGUGCUGCUCCUCAAGACGCU | |
| | GUACGACUCUGAGUAGCUGAUGUUGUUCCGGCGGGACUC | |
| | GUUCAAGUUCGUCCUGCACCUCUCGCGGGACGUGCACAA | |
| | GUUCUGGUGGCACUUGUCGGAGUAGUCGCUGGUCGACGA | |
| | GUACUCUUUGGUGGACUCUCUGGAGUACCCGCACGGGAU | |
| | GACGUUGAUGUCGUUCAAGACCAUAGACCUCGUGCGGUU | |
| | CUGGCCGCUCUGGUCGCACGGGUUCACGACCGACCACUG | |
| | GUUACCGUCGAUGGACUUGCUCUGGGUGAAGUCGCUGGU | |
| | CUAGCUCGUCCUUCGGCUGUUGUACUAGUGGCUCUACGA | |
| | CUCCUUCCUGAUGUAGUUCUCUGUCCCGUCGUGGGGGGA | |
| | CCGGGAGUACCUAGACGAGUACAAGUCGUGGUCGCGGAU | |
| | GGAGUAGUCGUAGAAGGACGUGGACCACUUCUAGGGGUG | |
| | GGUGUCUGUGUAGUUCCCGCCGUCGACGGGGUUCGGGGU | |
| | GUCUGAGUGGUUGUUCCCGUAGACGUCGACGCCGCGGAA | |
| | GUUCCACGGGCCGCACUUUUGGUAGACCUUCUCCUCUAU | |
| | UCGCCGGCGAUGCUGGAGCUGAUACUUUUUUUGAUUGUC | |
| | UAUAGGAGCUGCGGUGGUACUUGGCCUGUGACUGGGACA | |
| | AGUUGCACUGGGCCUUACUGCGGUCUCGGAUGCACAGGC | |
| | CGUAGGUCUUAUCGCACAGGCGGUUAUCUUCGCUGGGGC | |
| | ACUGUGACCUGCACGACGGACUAUCGUCGAUAGACUCGC | |
| | CGCGGUUGGACUUGGACUCGACGGUAAGACGGAGAGGUG | |
| | UCAUGUCGACCGCCUAGUUGCCUUAGGGAGUCGUCGUGU | |
| | GGGUCCACGACAAAUAGCGGUUCUAGUGGGGAUUGUUGU | |
| | UGCCGUGGAUGCGGACGAAGCACAGGUUAGACCGGUGGC | |
| | CGUCUUUGUUGUCGUAGCACUUCAGGUAGUGGCACAGAC | |
| | GGUCGCCGUGUAGAGGACCUGACUCGCGGUGUCUCAUGU | |
| | UCGACCACCAACACCCUCGACUGCCGCACCCUUUUUCGC | |
| | GGGACUGUUAGGUCGACUAGGUCUGGCUCAUAUUUGAGC | |
| | ACCAGCAGCCGCGACAACCGCAACCGUUUAGACGAGACU | |
| | GGUAAGUCGAGUAAGUCCACCGGCGACGGUCUGGACAAG | |
| | AAGGACGAGGACGGUAGUCGCUGACCCUUUCUCGAUCUU | |
| | CUUCCGUGCAGCCGGCCUUUCUUCCGCGGCGAUCUCGAU | |
| | CUCAAUCGGACGACGGUCUCAAACCGUGGCGGCUUCAAG | |
| | UAGACGGACGAGGAUCGCGACAGUCUCGGCGGUCUGGUC | |
| | CUAAACCGCCUCUAUUCCCGUCUGAGCACCCGCAACGUC | |
| | UUUCUGUCCACGGGACGUCUAAGGACUCCGGUUGUCCGC | |
| | ACGACUCGGACAAAGAACCGCCUCGACACCACAGGGACG | |
| | UCAUGCAGUCUGGAUCGCGAGACUCUUGGGACGACCUAG | |
| | UCUCGCGGUUCCUAACUAGUUGAUCGGUCUAAGAAGUAC | |
| | AAACCUGGUUUAGUUGAACACUAUGGUACGAGUUUCUCC | |
| | GGAGUUAAUAUAAACUCAAAAAUUAAAAAUACUUUUUUU | |
| | GAUUGUCGUUAGUACCUUCAGGUGCUAAAACUCUGGCUG | |
| | CUCAAGUUACUAAAGUUACUUCUACUGAUACGGUGUUCU | |
| | CUUAAGGACUUAGGGCUACUCGCGUACUGCAUGAACUUA | |
| | GUACGACUAAUGUUGGACUUAAGAGGAGAUUAAUCACUA | |
| | CUAUAACUGUUAAAUUAGUCCUUUAAGUUAAGAGAAGGU | |
| | UAAGGGAGCUACACCCUAUCAUUCUUGACCCUACCUCAA | |
| | GAACUCUACAAUUGCAGUACAGUUCGGUUAGGGUAGGGU | |
| | UGUAGAGUCUACGUAUUUACCUACCCUUCAACCAAUUAC | |
| | AGACUAUUAGUACUACGGUCAGUUCCCAUAUCAAAAAAU | |
| | GUACUUCACCUGUUUCUCCGUCUUUAUUGUAAACUGCAC | |
| | CACCUCUGGAAGUAGGCGCCGACCCCGUUGUUUGGUUAA | |
| | CUUAUGUAGUUUUUCCUUUCUACCUGACUGAGUAAGUUU | |
| | UAAGAGCGAAUAAACACAGUUUUCAAAAACCUGAAUGUG | |
| | UUCAACUGUAAUUAGAAUUUACGACAGAGACUCCACCUU | |
| | AACGAGUUGAACCGCUCCUGAAAGUUUCCGUUUCAGUCU | |
| | UCUUCAAGAGUACCUUGCUUGUAUACGUCCUAAUCCCAA | |
| | GGGUCGAACCCAGGAUGAAAAUAAAGUCUUCCUACCCGA | |
| | AUGAAGUUCUUUGAACUAUAAGAUUACCUGGCUUUGAAA | |
| | GACAAUUACCAGUUUCUACACUAAUAUCCCUCCUACGUU | |
| | UGCCACGAUAGGUACCAUACAUCUUAUCUGUUGGACAAG | |
| | AGUCUCGUUCUGUAGAAGAGGGAAGAUUUAUAGAUGUCU | |
| | UAACCUCUAUUUUAACACCUCUCCGUCCCUUUAAAAAGA | |
| | AUACUGAACUAAUUUUACCACCUUGGCUAUACGUUGAAC | |
| | UUCGACUACUUUAAUCGUUCUCUUAGUUCCGGAAAUCAG | |
| | GGUGUUAAGGGAGUAAAACUUUUAGUAUAGUUCUGAAGA | |
| | CAACUACUUCCCCGUUUUUAACUGGCUCCAUAUUCUAAG | |
| | GAGGUACUAGUCUAUUACUCACACUUUUGUCACCUAGAG | |
| | UGUGACCACUAAAUACCUAGCAAGUCUGUAACCCCAGUA | |
| | GGAAAAUAUCUAAUAAUGUGACCUGAUCUUUUUAAUGUA | |
| | AGGGUUCAUUGGUACUUCUUUCUAUAACUACACAGUAUA | |
| | CGUUUUCGUGAACGUUCACUAAAUCGAGCCUAACAAGAU | |
| | AAAGUUGUCAAGUUACUAGUAUUUUUCACCAAGCACUUA | |
| | CCUCUGAACGAGGGAGUACUAGUAGGGAAAUUUUCAGUA | |
| | CAAUUUCUUUUAUGUACCGGGUGUCGACGAGUUCAAGUU | |
| | CUAAAACCUCUAUUUACCGUACUUGAAGGCGACUAAUUU | |
| | ACAAAACUUUAUGGGCUGAAUGAUCUGGGUAGCUAUUAU | |
| | AUGAGACUGUUUUCAGUAAGUUACUUAUCCAGUCUCCAC | |
| | AACUUUGUACAGGCUUACUUAGGCUUGUGAGGAUAGGGA | |
| | UCAUUUUUCCACAACGUCUGAUACAACCUGUGUUUCCGA | |
| | UGGUUAACCUUUCUUAAAGAAUUUCUCUAACUACUCUUC | |
| | CCGAAUCUACUACUACUAGAUUAAUAACCAGAAUUUCCU | |
| | UUCCUCUCCCUUGACUUCAACCGUCCAUCUAAAAAGAGG | |
| | GAUUACAGAACCUUUAACGCUCUUAUGAAACAUUAAUGG | |
| | CUUAUAAACUAUUUCUGAGUAAAGCAGGGAUACAAAUUU | |
| | CCGGACUGUUACCGCCUGCUAGAUUGACGUCAGUAAUUU | |
| | UUCUACAAUCUAAGGAGUAGGCCGGUUCCUAACUUCAGU | |
| | AUACUCCGUUAAACGUAUCGGUUAGUGUAACUAAUGCUU | |
| | UUUACCUUAUUGGUGGUUUCCUUCAAUAGUUUGCCGGGU | |
| | CACAAGGCUCAAUACCCGGUCAAGAAUCCAAUAGGUAGG | |
| | AAUUAGCUCUCUUGAGUACUUAAAAAACUCUUUUCAGAA | |
| | UAUAUGAUGUUACCUUCUGGUCUGAACUACGCACAAGUG | |
| | UUGUUGUGUGACUAGUUAAGUUGGAGGGUUGCUCAAACA | |
| | ACCGUUCCUGUUCUCCCACCUGACCUUCCAGAUGCCGUU | |
| | UUUCCUACCUCAUAGGAGUUAGAUGACCAAUAAGUUUCU | |
| | CUCCGAUUUUAGUCUUUGUGACGACAGUUUCAGAACCGU | |
| | GUUCCACUAUUAGUUCAAUAAACGUGUGUCAUAUUUUGC | |
| | UUCUUUAGCUCUUUGCAACAUCUUAAUGUCCCACGAGAG | |
| | UUAGUUUACCAAAGAUUAUUACUCUUUUAAUACUGACGU | |
| | UAGUUUUAUCCCUGUCCCUUCAAUCCUGAAAACUAUUUA | |
| | CUGCUACUCUGAUACGUUAGACGUCUAAUGAACUUAAUA | |
| | CCUUUUUAUGGCUAAAAGGCACCUCACUAAUCUCCCAAU | |
| | CUCUGGUUCUCUACCAGUGCUCACUGAACACAGUGGUUA | |
| | CUGGUUUAUGGGUGAACACGAUUAUAUUACUCGAGUCAA | |
| | AGGUGUUUACGAGAGUGGCAUCGAGUAAAACGACUCUUG | |
| | GGUUAGUUACGGUACUAUGUCAUGUUAAUAAAACCCUGU | |
| | AAACGAUCUGAGAACAACUACUACGUACUAGGACGAGAA | |
| | GCAGUUAGUAACAUACUUCAAGUUCUAUUCUAUGGCCCG | |
| | AACGUGUCAAGAUGAAAGUUUAUGCGGUACAACAUAAAC | |
| | CUGGGAAGGUAACCUCCUCACAGCCCGUACAGAAACAGG | |
| | UCCAAAAACUAAUCUCGGAAGGGUCUAGGGCAUUGUCUU | |
| | UCAGAGAGUAAGACCUCUAAGUAGGUACAUGUACGAGCU | |
| | UCACUCGUAGACUUCCUCUACUCACGUCAUAAACCUUUG | |
| | GGGCUCUAUCGGUUCAAAGCUUAUUGAGUGUAUCUGUUC | |
| | GAUCAUCUUCUAGGUUGGAGAGACUUGUAGCGAUACCCU | |
| | UACUCAGGUCGCUUGAACAAUUUCUGACUCCAAUUUUUU | |
| | ACGAAUUAGCUUAGUUCUGUUUGGUAGUCCUUGGUCCAC | |
| | UAAUUCCUACGUUGGUAUAUAAACAUAGUACUUCUCCUA | |
| | GCCGAGUCUUCAAAGAAUACCAGUUAUUUAGGAGACAAG | |
| | GGAUCUAAAAAUUCACUUAAGUUUAGUCCGUGAAAAAAC | |
| | CCUCAGCGUCUGCCCGAGUAGUCAGAUAAAGUUUUAAGA | |
| | GCAUGAUAAGCCUUGAGGAAAUUCUUUUUCAUAGUAUCC | |
| | CUUAACCUACUAAACUAACACUCCUCACUCCAUAGGAGA | |
| | AACUGUGUAAAUCCCUUUGAAGUAAACUCUUCCCCUAGU | |
| | ACAUUUUACACCUGUACAAGUCGAUGAGUACGACUGUGU | |
| | AAUUCUAUGUUUAGGACCCCGGCAUGUCAAUAACCCUGU | |
| | UGACAUGGGGUAGGUAAUCUUUACAACCCAGGUGUUGUA | |
| | GCUUUUCUCUGAGGAACACGUGGUACAUUGUGUAGUCCC | |
| | AAGUUAAUACAAAGACACGUAACAGGUCUGCCCUAGGUA | |
| | CUGCAGAAAUCAAGUGCCCCUGGUAACGGACGAAUAGAU | |
| | CCCAGAUUUUGUAGACUUAGAUGUAGAUAAAACGUCGGA | |
| | ACCCUUUCCCUUUCGUUUCAGGGUGACUAAUUUUCUCGA | |
| | UGUGCAGAAUCUCUACGAUAGAGAACCAAACAACUUGGG | |
| | CUGAGAUUUGAUCGUUACUGAUAUGAAAGAUUGUAGGUG | |
| | AGAAAUUGUCCGCUUCUUACCUGGUUUUCCGUCGUACCC | |
| | AAGUUUUCUUGUCCCAGACGGGAAGUAUCCAAAAGCUGU | |
| | AGAGCCUACUCGGUACCACCCAAGCGUAGAGUCUCGUGA | |
| | CGUCGUAACUGGUCCAACUACCGUUGAUGUCUGUGGUAC | |
| | UCCCUAGACCCUCUAGUCUUAAAGCUGAAAAAUAAGGUU | |
| | CGUUGCAACGAGAUACGAGUUUAAUGGUGGUGACAACGU | |
| | UCUCUGCCUACCUAGUGGUCAACAUGUCUAGUAAUAGUA | |
| | UAACGGACAUUCAGGACAAACUCUGGGUAUCUUCUCUAG | |
| | UGGGACCUGAGUUCAUACCUGAUGUGCGGGGGUCUACAU | |
| | AGGGUACACGACUUCUGUACCUCCUUACCCCUUCCAAGC | |
| | ACCCCUGUUCUCUAUUUUGUCUAGAUAGGAAAUCUUCCC | |
| | UUAACCUUCUUAAAUCGUGGACGACUCGUUAGGAUAGUU | |
| | CAGCCGUCUACAUAUCCAAAAGAUAUACCUCUGAACCGC | |
| | AUAUCUUUUAGAUGAGUACGGCUCCUGUCAAGAGAUAAA | |
| | GGAGAUAGAUAUGUUCCAGCAUAAUCUCCAGCUCCAAAG | |
| | AAUUUUCCCAACGAUCUGCCUAAUUACUCUCGUUCAACG | |
| | ACGGUUCAUUAUGUGGCCUCUUCAGACCGAGUAAACUUC | |
| | UCCGGCCGGUUGCGUCACAUGCCUCCAAACUAAAUGAAC | |
| | UAACUAUUUAACUCACAUAGUGGAGGUAAGGAAAGAGAA | |
| | UGAUCUAGUCCUGGAUAAUCUCUGCUUAAUCUUUGCUAA | |
| | GGGGUGUUCUAGGGUUGGAGGAUAGGCUGUUCGUUGGCA | |
| | CUAUACCCCCACUAACAGUCUUUAAUGAAGUUUAUGGUU | |
| | ACGGCAGAUUAACUUUUCCCUUUUAUGUCUAGUGUAAUA | |
| | AGUGUUAAUACCAAUAAGAGUCUACAGAAUAGGUAUCUG | |
| | AAGUAACCUGGUAAGAGAUAAAGGUGGUGGGAGAACGUU | |
| | UAGGAUAUGUUCGGUAAAAAUAGACCCUUUCUAUUCUUA | |
| | CUCAACUCUCUCGACCGUUUAGAAAGAAGUAACGAUUCU | |
| | AGUCCUCUCCCCACCCUUCUGUAUGUACACUUUAAGAAG | |
| | UGGUUCCUGUAUAAUAACACAGGUCUCCUUUAGUCUGUA | |
| | CGAACGUUCAAGCCCUAACGAUUCCUAUUAUUAUUUCUG | |
| | UACUCGAUAGGGGGAACCCCUUCCCUUAGGUCUCCCUGU | |
| | UAAUGUUGUUAGGGACAAAUAAUAUGCUGGUGGGGAAUG | |
| | GGUUUCUACGAUCUCUACGGAGGUUCUUAGGUUUUAGGG | |
| | GACGACAGGCCUUAGUCCAACCCGGUUAAUGGUUGACCG | |
| | CGAGUAAUAUUUUAAGCCUCAUAUAAUGUACCUUACCCU | |
| | UAGGUAAUGUCCCUGAAGAACUCAACACCUCUGCCGAGG | |
| | CCUCCCUACUGACGACGUAAUGAUGCUCUUUUACACGUA | |
| | UCGUCUCCUUAUAAGUUAUCAGACAAUCUUAAUAGUCCC | |
| | AGUCAGUACGCUCCGCGGAGAGGACUCGGGGGGUCACGG | |
| | GAUCUUUGAAAUCCUCCUCUAUUUAGCUCUACACAUUUA | |
| | CCACUUUGUACAACCCUUAUAGGUAGACUGAAUACACUG | |
| | GGUUCCUGAACCCUGAUAAAGGAGGCUGAGUUUCGUCCG | |
| | AACCCCGAAGUUUAACUAAAUUAACAUUACCUAUACCUU | |
| | CAAGCCCUAAGAAGAUGAUCGGACUUUUAACUCUGCUUA | |
| | CAAUCUUUAAUACACGUGGCCUAAAACCUACUCGUUCCU | |
| | CAAAAUUAGAUGUUCUGAAUACCUUGUAUAUAAACACUC | |
| | UCGCUUUUCUUACGUCAUUGUUAGGAACCAGGGUACAAG | |
| | UUCUGCCAGCUGAAUCAAGUUUGUCUUAAAUCAUCAAGA | |
| | GUUUGCAGACUUCAUAUAUACCAUACAUUUCCAAACUUC | |
| | UUUAAUUAGCUACUUGGGUUAGGGCUAACCAGAAGGUAG | |
| | UUACUUAGGACCUUUUUGGACAUGCGUAAGGUCAGUAGU | |
| | CUUGUCCUUAAACGGUCUCGUUUCUUCCAAUCAUGUAUG | |
| | AAAUGGAACUGUCCAUAAGGGAGGGUUAAGUAAGGACUA | |
| | GGAAAACAUUUGUAACUCUGAUACGAUGUUUAUAAGCCU | |
| | CAUGGGUGCCCACACAGAGUACGCCGACGGAAUUUUAGU | |
| | AGACUAUCUGGACGUCUAAAUAACUGGUAAUCGGAAAAA | |
| | AUAUACCGCUAAUAUAGCAUAAUAUUGUAGUUAGUAUAG | |
| | UCUCAUCCUGGCUAUGGAGGCUUGGGGGGUAGUCUACCU | |
| | UAACGUGUUUUACACCCCUAGCGAUAUUGACCAUAUUCG | |
| | AAAACCGACUCAAACUACCUCUUUCUGUAAGGUGAUAUA | |
| | GUUGUCACAAAUCGUCAAUAGGUCGUUAGUAAGGGCUAA | |
| | UCCACCCUCCGACAAAGUCAUUUUCCUCCUAUGUUCGUC | |
| | UUCACCUCAUGAUCUCCACUACCCGAGGGUUUUCUAUGG | |
| | GCUUAAAGUCUGAGGAACCGGGGUUAGCCCUUGACCUAG | |
| | UCUAGAGACCUUAACCAGGCUUUGGUUCAAGCAGAUUUA | |
| | GGUAAGUUACUCUAGAACAAGUUAGUCGAUACAGCAUGU | |
| | CACCUAUUAGUAAACUUUACCAGUUUAAACGCUUCUUUG | |
| | UGUCCUUACUAACUUACCUAGUUAUCUGCUUAAAGUUUU | |
| | CUUCUGGCCAGAUAUGACUACAACUUCUCACUGGAUGUG | |
| | CUCCUUUUGAGAACCUCUCUAAUUUUUUAGUACUCCUCU | |
| | GAGGUUUGAAAUUCAUACUUUUUUUGAAACUAGGAAUUC | |
| | UGGGAGAACACCAAAAAUAAAAAAUAGACCAAAACACCA | |
| | GAAGCA | |
| SEQ ID NO: 31 | EVMLVESGGGLVQPGGSLRLSCTASGFTFSASAMSWVRQ | heavy chain |
| | APGKGLEWVAYISGGGGDTYYSSSVKGRFTISRDNAKNS | of anti-PD1 |
| | LYLQMNSLRAEDTAVYYCARHSNVNYYAMDYWGQGTLVT | antibody |
| | VSSASTKGPSVFPLAPCSRSTSESTAALGCLVKDYFPEP | PD1-1 |
| | VTVSWNSGALTSGVHTFPAVLQSSGLYSLSSVVTVPSSS | |
| | LGTKTYTCNVDHKPSNTKVDKRVESKYGPPCPPCPAPEF | |
| | LGGPSVFLFPPKPKDTLMISRTPEVTCVVVDVSQEDPEV | |
| | QFNWYVDGVEVHNAKTKPREEQFNSTYRVVSVLTVLHQD | |
| | WLNGKEYKCKVSNKGLPSSIEKTISKAKGQPREPQVYTL | |
| | PPSQEEMTKNQVSLTCLVKGFYPSDIAVEWESNGQPENN | |
| | YKTTPPVLDSDGSFFLYSRLTVDKSRWQEGNVFSCSVMH | |
| | EALHNHYTQKSLSLSLG | |
| SEQ ID NO: 32 | EIVLTQSPATLSLSPGERATMSCRASENIDTSGISFMNW | light chain |
| | YQQKPGQAPKLLIYVASNQGSGIPARFSGSGSGTDFTLT | of anti-PD1 |
| | ISRLEPEDFAVYYCQQSKEVPWTFGQGTKLEIKRTVAAP | antibody |
| | SVFIFPPSDEQLKSGTASVVCLLNNFYPREAKVQWKVDN | PD1-1 |
| | ALQSGNSQESVTEQDSKDSTYSLSSTLTLSKADYEKHKV | |
| | YACEVTHQGLSSPVTKSFNRGEC | |
| SEQ ID NO: 33 | EVMLVESGGGLVQPGGSLRLSCTASGFTFSASAMSWVRQ | heavy chain |
| | APGKGLEWVAYISGGGGDTYYSSSVKGRFTISRDNAKNS | of anti-PD1 |
| | LYLQMNSLRAEDTAVYYCARHSNPNYYAMDYWGQGTLVT | antibody |
| | VSSASTKGPSVFPLAPCSRSTSESTAALGCLVKDYFPEP | PD1-2 |
| | VTVSWNSGALTSGVHTFPAVLQSSGLYSLSSVVTVPSSS | |
| | LGTKTYTCNVDHKPSNTKVDKRVESKYGPPCPPCPAPEF | |
| | LGGPSVFLFPPKPKDTLMISRTPEVTCVVVDVSQEDPEV | |
| | QFNWYVDGVEVHNAKTKPREEQFNSTYRVVSVLTVLHQD | |
| | WLNGKEYKCKVSNKGLPSSIEKTISKAKGQPREPQVYTL | |
| | PPSQEEMTKNQVSLTCLVKGFYPSDIAVEWESNGQPENN | |
| | YKTTPPVLDSDGSFFLYSRLTVDKSRWQEGNVFSCSVMH | |
| | EALHNHYTQKSLSLSLG | |
| SEQ ID NO: 34 | EIVLTQSPATLSLSPGERATMSCRASENIDTSGISFMNW | light chain |
| | YQQKPGQAPKLLIYVASNQGSGIPARFSGSGSGTDFTLT | of anti-PD1 |
| | ISRLEPEDFAVYYCQQSKEVPWTFGQGTKLEIKRTVAAP | antibody |
| | SVFIFPPSDEQLKSGTASVVCLLNNFYPREAKVQWKVDN | PD1-2 |
| | ALQSGNSQESVTEQDSKDSTYSLSSTLTLSKADYEKHKV | |
| | YACEVTHQGLSSPVTKSFNRGEC | |
| SEQ ID NO: 35 | EVMLVESGGGLVQPGGSLRLSCTASGFTFSKSAMSWVRQ | heavy chain |
| | APGKGLEWVAYISGGGGDTYYSSSVKGRFTISRDNAKNS | of anti-PD1 |
| | LYLQMNSLRAEDTAVYYCARHSNVNYYAMDYWGQGTLVT | antibody |
| | VSSASTKGPSVFPLAPCSRSTSESTAALGCLVKDYFPEP | PD1-3 |
| | VTVSWNSGALTSGVHTFPAVLQSSGLYSLSSVVTVPSSS | |
| | LGTKTYTCNVDHKPSNTKVDKRVESKYGPPCPPCPAPEF | |
| | LGGPSVFLFPPKPKDTLMISRTPEVTCVVVDVSQEDPEV | |
| | QFNWYVDGVEVHNAKTKPREEQFNSTYRVVSVLTVLHQD | |
| | WLNGKEYKCKVSNKGLPSSIEKTISKAKGQPREPQVYTL | |
| | PPSQEEMTKNQVSLTCLVKGFYPSDIAVEWESNGQPENN | |
| | YKTTPPVLDSDGSFFLYSRLTVDKSRWQEGNVFSCSVMH | |
| | EALHNHYTQKSLSLSLG | |
| SEQ ID NO: 36 | EIVLTQSPATLSLSPGERATMSCRASENIDVSGISFMNW | light chain |
| | YQQKPGQAPKLLIYVASNQGSGIPARFSGSGSGTDFTLT | of anti-PD1 |
| | ISRLEPEDFAVYYCQQSKEVPWTFGQGTKLEIKRTVAAP | antibody |
| | SVFIFPPSDEQLKSGTASVVCLLNNFYPREAKVQWKVDN | PD1-3 |
| | ALQSGNSQESVTEQDSKDSTYSLSSTLTLSKADYEKHKV | |
| | YACEVTHQGLSSPVTKSFNRGEC | |
| SEQ ID NO: 37 | GTGGCCGCTGCCAGACCTGTTCTTCCTGCTCCTGCCATC | KRAS-uORF1 |
| | AGCGACTGGGAAAGAGCTAGAAGAAGGCAC | |
| SEQ ID NO: 38 | GTCGGCCGGAAAGAAGGCGCCGCTAGAGCTAGAGTTAGC | TPX2-uORF1 |
| | CTGCTGCCAGAGTTTGGCACCGCCGAAGTTCATCTGCCT | |
| | GCTCCTAGCGCTGTCAGAGCCGCCAGACCAGGATTT | |
| SEQ ID NO: 39 | GGCGGAGATAAGGGCAGACTCGTGGGCGTTGCAGAAAGA | AURKA-uORF2 |
| | CAGGTGCCCTGCAGATTCCTGAGGCCA | |
| SEQ ID NO: 40 | AACCGGACACTGACCCTGTTCAACGTGACCCGGAATGAC | CEACAM5 |
| | GCCAGAGCCTACGTGTCCGGCATCCAGAATAGCGTGTCC | fragment |
| | GCCAATAGAAGCGACCCCGTGACACTGGACGTGCTG | |
| SEQ ID NO: 41 | CCTGATAGCAGCTATCTGAGCGGCGCCAACCTGAACCTG | CEACAM5 |
| | AGCTGCCATTCTGCCTCT | fragment |
| SEQ ID NO: 42 | CCACAGTACAGCTGGCGGATCAACGGAATCCCTCAGCAG | CEACAM5 |
| | CACACCCAGGTGCTGTTTATCGCCAAGATCACCCCTAAC | fragment |
| | AACAACGGCACCTACGCCTGCTTCGTGTCCAATCTGGCC | |
| | ACCGGCAGAAACAACAGCATCGTGAAGTCCATCACCGTG | |
| | TCTGCCAGCGGCACATCTCCTGGACTGAGCGCC | |
| SEQ ID NO: 43 | GGCGGAGATAAGGGCAGACTCGTGGGCGTTGCAGAAAGA | DUOXA2 |
| | CAGGTGCCCTGCAGATTCCTGAGGCCA | fragment |
| SEQ ID NO: 44 | ACAGAGTACAAGCTGGTGGTTGTGGGAGCTGACGGCGTG | KRAS-G12D |
| | GGAAAAAGCGCCCTGACAATCCAGCTGATCCAG | fragment |
| SEQ ID NO: 45 | ACCGAGTATAAACTCGTGGTCGTCGGCGCTGTTGGCGTT | KRAS-G12V |
| | GGCAAATCTGCTCTGACCATTCAGCTCATTCAG | fragment |
| SEQ ID NO: 46 | KRYKNRVASRKSRAKFKQLLQHYREVAAAKSSENDRLRL | ATP150 (v2) |
| | LLKNRTLTLFNVTRNDARAYVSGIQNSVSANRSDPVTLD | |
| | VLPDSSYLSGANLNLSCHSASPQYSWRINGIPQQHTQVL | |
| | FIAKITPNNNGTYACFVSNLATGRNNSIVKSITVSASGT | |
| | SPGLSATEYKLVVVGADGVGKSALTIQLIQVAAARPVLP | |
| | APAISDWERARRRHVGRKEGAARARVSLLPEFGTAEVHL | |
| | PAPSAVRAARPGFGGDKGRLVGVAERQVPCRFLRPTGVL | |
| | SLFLGGAVVSLQYVRPSALRTLLDQSAKDSTVHEILSKL | |
| | SLEGDHSTPPSAYGSVKPYTNFDAE | |
| SEQ ID NO: 47 | KRYKNRVASRKSRAKFKQLLQHYREVAAAKSSENDRLRL | ATP152 (v2) |
| | LLKNRTLTLFNVTRNDARAYVSGIQNSVSANRSDPVTLD | |
| | VLPDSSYLSGANLNLSCHSASPQYSWRINGIPQQHTQVL | |
| | FIAKITPNNNGTYACFVSNLATGRNNSIVKSITVSASGT | |
| | SPGLSATEYKLVVVGAVGVGKSALTIQLIQVAAARPVLP | |
| | APAISDWERARRRHVGRKEGAARARVSLLPEFGTAEVHL | |
| | PAPSAVRAARPGFGGDKGRLVGVAERQVPCRFLRPTGVL | |
| | SLFLGGAVVSLQYVRPSALRTLLDQSAKDSTVHEILSKL | |
| | SLEGDHSTPPSAYGSVKPYTNFDAE | |
| SEQ ID NO: 48 | NRTLTLFNVTRNDARAYVSGIQNSVSANRSDPVTLDVLP | multi- |
| | DSSYLSGANLNLSCHSASPQYSWRINGIPQQHTQVLFIA | antigenic |
| | KITPNNNGTYACFVSNLATGRNNSIVKSITVSASGTSPG | domain |
| | LSATEYKLVVVGADGVGKSALTIQLIQTEYKLVVVGAVG | VSV-GP154 |
| | VGKSALTIQLIQVAAARPVLPAPAISDWERARRRHVGRK | (v2) |
| | EGAARARVSLLPEFGTAEVHLPAPSAVRAARPGFGGDKG | |
| | RLVGVAERQVPCRFLRPTGVLSLFLGGAVVSLQYVRPSA | |
| | LRTLLDQSAKD | |
| SEQ ID NO: 49 | ACGAAGACAAACAAACCATTATTATCATTAAAAGGCTCA | cDNA |
| | GGAGAAACTTTAACAGTAATCAAAATGTCTGTTACAGTC | sequence of |
| | AAGAGAATCATTGACAACACAGTCGTAGTTCCAAAACTT | VSV-GP154 |
| | CCTGCAAATGAGGATCCAGTGGAATACCCGGCAGATTAC | |
| | TTCAGAAAATCAAAGGAGATTCCTCTTTACATCAATACT | |
| | ACAAAAAGTTTGTCAGATCTAAGAGGATATGTCTACCAA | |
| | GGCCTCAAATCCGGAAATGTATCAATCATACATGTCAAC | |
| | AGCTACTTGTATGGAGCATTAAAGGACATCCGGGGTAAG | |
| | TTGGATAAAGATTGGTCAAGTTTCGGAATAAACATCGGG | |
| | AAAGCAGGGGATACAATCGGAATATTTGACCTTGTATCC | |
| | TTGAAAGCCCTGGACGGCGTACTTCCAGATGGAGTATCG | |
| | GATGCTTCCAGAACCAGCGCAGATGACAAATGGTTGCCT | |
| | TTGTATCTACTTGGCTTATACAGAGTGGGCAGAACACAA | |
| | ATGCCTGAATACAGAAAAAAGCTCATGGATGGGCTGACA | |
| | AATCAATGCAAAATGATCAATGAACAGTTTGAACCTCTT | |
| | GTGCCAGAAGGTCGTGACATTTTTGATGTGTGGGGAAAT | |
| | GACAGTAATTACACAAAAATTGTCGCTGCAGTGGACATG | |
| | TTCTTCCACATGTTCAAAAAACATGAATGTGCCTCGTTC | |
| | AGATACGGAACTATTGTTTCCAGATTCAAAGATTGTGCT | |
| | GCATTGGCAACATTTGGACACCTCTGCAAAATAACCGGA | |
| | ATGTCTACAGAAGATGTAACGACCTGGATCTTGAACCGA | |
| | GAAGTTGCAGATGAAATGGTCCAAATGATGCTTCCAGGC | |
| | CAAGAAATTGACAAGGCCGATTCATACATGCCTTATTTG | |
| | ATCGACTTTGGATTGTCTTCTAAGTCTCCATATTCTTCC | |
| | GTCAAAAACCCTGCCTTCCACTTCTGGGGGCAATTGACA | |
| | GCTCTTCTGCTCAGATCCACCAGAGCAAGGAATGCCCGA | |
| | CAGCCTGATGACATTGAGTATACATCTCTTACTACAGCA | |
| | GGTTTGTTGTACGCTTATGCAGTAGGATCCTCTGCCGAC | |
| | TTGGCACAACAGTTTTGTGTTGGAGATAACAAATACACT | |
| | CCAGATGATAGTACCGGAGGATTGACGACTAATGCACCG | |
| | CCACAAGGCAGAGATGTGGTCGAATGGCTCGGATGGTTT | |
| | GAAGATCAAAACAGAAAACCGACTCCTGATATGATGCAG | |
| | TATGCGAAAAGAGCAGTCATGTCACTGCAAGGCCTAAGA | |
| | GAGAAGACAATTGGCAAGTATGCTAAGTCAGAATTTGAC | |
| | AAATGACCCTATAATTCTCAGATCACCTATTATATATTA | |
| | TGCTACATATGAAAAAAACTAACAGATATCATGGATAAT | |
| | CTCACAAAAGTTCGTGAGTATCTCAAGTCCTATTCTCGT | |
| | CTGGATCAGGCGGTAGGAGAGATAGATGAGATCGAAGCA | |
| | CAACGAGCTGAAAAGTCCAATTATGAGTTGTTCCAAGAG | |
| | GATGGAGTGGAAGAGCATACTAAGCCCTCTTATTTTCAG | |
| | GCAGCAGATGATTCTGACACAGAATCTGAACCAGAAATT | |
| | GAAGACAATCAAGGCTTGTATGCACCAGATCCAGAAGCT | |
| | GAGCAAGTTGAAGGCTTTATACAGGGGCCTTTAGATGAC | |
| | TATGCAGATGAGGAAGTGGATGTTGTATTTACTTCGGAC | |
| | TGGAAACAGCCTGAGCTTGAATCTGACGAGCATGGAAAG | |
| | ACCTTACGGTTGACATCGCCAGAGGGTTTAAGTGGAGAG | |
| | CAGAAATCCCAGTGGCTTTCGACGATTAAAGCAGTCGTG | |
| | CAAAGTGCCAAATACTGGAATCTGGCAGAGTGCACATTT | |
| | GAAGCATCGGGAGAAGGGGTCATTATGAAGGAGCGCCAG | |
| | ATAACTCCGGATGTATATAAGGTCACTCCAGTGATGAAC | |
| | ACACATCCGTCCCAATCAGAAGCAGTATCAGATGTTTGG | |
| | TCTCTCTCAAAGACATCCATGACTTTCCAACCCAAGAAA | |
| | GCAAGTCTTCAGCCTCTCACCATATCCTTGGATGAATTG | |
| | TTCTCATCTAGAGGAGAGTTCATCTCTGTCGGAGGTGAC | |
| | GGACGAATGTCTCATAAAGAGGCCATCCTGCTCGGCCTG | |
| | AGATACAAAAAGTTGTACAATCAGGCGAGAGTCAAATAT | |
| | TCTCTGTAGACTATGAAAAAAAGTAACAGATATCACGAT | |
| | CTAAGTGTTATCCCAATCCATTCATCATGAGTTCCTTAA | |
| | AGAAGATTCTCGGTCTGAAGGGGAAAGGTAAGAAATCTA | |
| | AGAAATTAGGGATCGCACCACCCCCTTATGAAGAGGACA | |
| | CTAGCATGGAGTATGCTCCGAGCGCTCCAATTGACAAAT | |
| | CCTATTTTGGAGTTGACGAGATGGACACCTATGATCCGA | |
| | ATCAATTAAGATATGAGAAATTCTTCTTTACAGTGAAAA | |
| | TGACGGTTAGATCTAATCGTCCGTTCAGAACATACTCAG | |
| | ATGTGGCAGCCGCTGTATCCCATTGGGATCACATGTACA | |
| | TCGGAATGGCAGGGAAACGTCCCTTCTACAAAATCTTGG | |
| | CTTTTTTGGGTTCTTCTAATCTAAAGGCCACTCCAGCGG | |
| | TATTGGCAGATCAAGGTCAACCAGAGTATCACGCTCACT | |
| | GCGAAGGCAGGGCTTATTTGCCACATAGGATGGGGAAGA | |
| | CCCCTCCCATGCTCAATGTACCAGAGCACTTCAGAAGAC | |
| | CATTCAATATAGGTCTTTACAAGGGAACGATTGAGCTCA | |
| | CAATGACCATCTACGATGATGAGTCACTGGAAGCAGCTC | |
| | CTATGATCTGGGATCATTTCAATTCTTCCAAATTTTCTG | |
| | ATTTCAGAGAGAAGGCCTTAATGTTTGGCCTGATTGTCG | |
| | AGAAAAAGGCATCTGGAGCGTGGGTCCTGGACTCTATCG | |
| | GCCACTTCAAATGAGCTAGTCTAACTTCTAGCTTCTGAA | |
| | CAATCCCCGGTTTACTCAGTCTCCCCTAATTCCAGCCTC | |
| | TCGAACAACTAATATCCTGTCTTTTCTATCCCTATGAAA | |
| | AAAACTAACAGAGATCGATCTGTTTACGCGTCACGGATC | |
| | CCCCGGGCTGCAGGAATTCGCCACCATGGGCCAGATCGT | |
| | GACCATGTTCGAGGCCCTGCCCCACATCATCGACGAGGT | |
| | GATCAACATCGTGATCATCGTGCTCATCATCATCACCAG | |
| | CATCAAGGCCGTGTACAACTTCGCCACCTGCGGCATCCT | |
| | GGCCCTGGTGAGCTTCCTGTTCCTGGCCGGCAGAAGCTG | |
| | CGGCATGTACGGCCTGAATGGCCCCGATATCTACAAGGG | |
| | CGTGTACCAGTTCAAGAGCGTGGAGTTCGACATGAGCCA | |
| | CCTGAACCTCACCATGCCCAACGCCTGCAGCGCCAACAA | |
| | TAGCCACCACTACATCAGCATGGGCAGCAGCGGCCTGGA | |
| | GTTGACCTTCACCAACGACAGCATCCTGAACCACAACTT | |
| | CTGCAACCTCACCAGCGCCTTCAACAAGAAAACCTTCGA | |
| | CCACACCCTCATGAGCATCGTGAGCAGCCTGCACCTGAG | |
| | CATCAGAGGCAACAGCAACCACAAGGCCGTGAGCTGCGA | |
| | CTTCAACAACGGCATCACCATCCAGTACAACCTGAGCTT | |
| | CAGCGATCCTCAGAGCGCCATCAGCCAGTGCAGAACCTT | |
| | CAGAGGCAGAGTGCTGGACATGTTCAGAACCGCCTTCGG | |
| | CGGCAAGTACATGAGAAGCGGCTGGGGCTGGGCCGGCAG | |
| | CGACGGCAAGACCACCTGGTGCAGCCAGACCAGCTACCA | |
| | GTACCTCATCATCCAGAACAGAACCTGGGAGAACCACTG | |
| | CAGATACGCCGGACCTTTCGGCATGAGCAGAATCCTGTT | |
| | CGCCCAGGAGAAAACCAAGTTCCTCACCAGGAGACTGGC | |
| | CGGCACCTTCACCTGGACCCTGAGCGACAGCAGCGGCGT | |
| | GGAGAACCCTGGCGGCTACTGCCTCACCAAGTGGATGAT | |
| | CCTGGCCGCCGAGCTGAAGTGCTTCGGCAACACCGCCGT | |
| | GGCCAAGTGCAACGTGAACCACGACGAGGAGTTCTGCGA | |
| | CATGCTGAGACTCATCGACTACAACAAGGCCGCCCTGAG | |
| | CAAGTTCAAGCAGGACGTGGAGAGCGCCCTGCACGTGTT | |
| | CAAGACCACCGTGAACAGCCTCATCAGCGACCAGCTGCT | |
| | CATGAGAAACCACCTGAGAGACCTCATGGGCGTGCCCTA | |
| | CTGCAACTACAGCAAGTTCTGGTATCTGGAGCACGCCAA | |
| | GACCGGCGAGACCAGCGTGCCCAAGTGCTGGCTGGTGAC | |
| | CAATGGCAGCTACCTGAACGAGACCCACTTCAGCGACCA | |
| | GATCGAGCAGGAAGCCGACAACATGATCACCGAGATGCT | |
| | GAGGAAGGACTACATCAAGAGACAGGGCAGCACCCCCCT | |
| | GGCCCTCATGGATCTGCTCATGTTCAGCACCAGCGCCTA | |
| | CCTCATCAGCATCTTCCTGCACCTGGTGAAGATCCCCAC | |
| | CCACAGACACATCAAGGGCGGCAGCTGCCCCAAGCCCCA | |
| | CAGACTCACCAACAAGGGCATCTGCAGCTGCGGCGCCTT | |
| | CAAGGTGCCCGGCGTGAAAACCATCTGGAAGAGGAGATA | |
| | AGCGGCCGCTACGACCTCGACTATGAAAAAAACTAACAG | |
| | ATATCCTCGACGCCACCATGAACCGGACACTGACCCTGT | |
| | TCAACGTGACCCGGAATGACGCCAGAGCCTACGTGTCCG | |
| | GCATCCAGAATAGCGTGTCCGCCAATAGAAGCGACCCCG | |
| | TGACACTGGACGTGCTGCCTGATAGCAGCTATCTGAGCG | |
| | GCGCCAACCTGAACCTGAGCTGCCATTCTGCCTCTCCAC | |
| | AGTACAGCTGGCGGATCAACGGAATCCCTCAGCAGCACA | |
| | CCCAGGTGCTGTTTATCGCCAAGATCACCCCTAACAACA | |
| | ACGGCACCTACGCCTGCTTCGTGTCCAATCTGGCCACCG | |
| | GCAGAAACAACAGCATCGTGAAGTCCATCACCGTGTCTG | |
| | CCAGCGGCACATCTCCTGGACTGAGCGCCACAGAGTACA | |
| | AGCTGGTGGTTGTGGGAGCTGACGGCGTGGGAAAAAGCG | |
| | CCCTGACAATCCAGCTGATCCAGACCGAGTATAAACTCG | |
| | TGGTCGTCGGCGCTGTTGGCGTTGGCAAATCTGCTCTGA | |
| | CCATTCAGCTCATTCAGGTGGCCGCTGCCAGACCTGTTC | |
| | TTCCTGCTCCTGCCATCAGCGACTGGGAAAGAGCTAGAA | |
| | GAAGGCACGTCGGCCGGAAAGAAGGCGCCGCTAGAGCTA | |
| | GAGTTAGCCTGCTGCCAGAGTTTGGCACCGCCGAAGTTC | |
| | ATCTGCCTGCTCCTAGCGCTGTCAGAGCCGCCAGACCAG | |
| | GATTTGGCGGAGATAAGGGCAGACTCGTGGGCGTTGCAG | |
| | AAAGACAGGTGCCCTGCAGATTCCTGAGGCCAACAGGCG | |
| | TGCTGAGCCTGTTTCTTGGCGGAGCTGTGGTGTCCCTGC | |
| | AGTACGTCAGACCTAGCGCTCTGAGAACCCTGCTGGATC | |
| | AGAGCGCCAAGGATTGATCAACTAGCCAGATTCTTCATG | |
| | TTTGGACCAAATCAACTTGTGATACCATGCTCAAAGAGG | |
| | CCTCAATTATATTTGAGTTTTTAATTTTTATGAAAAAAA | |
| | CTAACAGCAATCATGGAAGTCCACGATTTTGAGACCGAC | |
| | GAGTTCAATGATTTCAATGAAGATGACTATGCCACAAGA | |
| | GAATTCCTGAATCCCGATGAGCGCATGACGTACTTGAAT | |
| | CATGCTGATTACAACCTGAATTCTCCTCTAATTAGTGAT | |
| | GATATTGACAATTTAATCAGGAAATTCAATTCTCTTCCA | |
| | ATTCCCTCGATGTGGGATAGTAAGAACTGGGATGGAGTT | |
| | CTTGAGATGTTAACGTCATGTCAAGCCAATCCCATCCCA | |
| | ACATCTCAGATGCATAAATGGATGGGAAGTTGGTTAATG | |
| | TCTGATAATCATGATGCCAGTCAAGGGTATAGTTTTTTA | |
| | CATGAAGTGGACAAAGAGGCAGAAATAACATTTGACGTG | |
| | GTGGAGACCTTCATCCGCGGCTGGGGCAACAAACCAATT | |
| | GAATACATCAAAAAGGAAAGATGGACTGACTCATTCAAA | |
| | ATTCTCGCTTATTTGTGTCAAAAGTTTTTGGACTTACAC | |
| | AAGTTGACATTAATCTTAAATGCTGTCTCTGAGGTGGAA | |
| | TTGCTCAACTTGGCGAGGACTTTCAAAGGCAAAGTCAGA | |
| | AGAAGTTCTCATGGAACGAACATATGCAGGATTAGGGTT | |
| | CCCAGCTTGGGTCCTACTTTTATTTCAGAAGGATGGGCT | |
| | TACTTCAAGAAACTTGATATTCTAATGGACCGAAACTTT | |
| | CTGTTAATGGTCAAAGATGTGATTATAGGGAGGATGCAA | |
| | ACGGTGCTATCCATGGTATGTAGAATAGACAACCTGTTC | |
| | TCAGAGCAAGACATCTTCTCCCTTCTAAATATCTACAGA | |
| | ATTGGAGATAAAATTGTGGAGAGGCAGGGAAATTTTTCT | |
| | TATGACTTGATTAAAATGGTGGAACCGATATGCAACTTG | |
| | AAGCTGATGAAATTAGCAAGAGAATCAAGGCCTTTAGTC | |
| | CCACAATTCCCTCATTTTGAAAATCATATCAAGACTTCT | |
| | GTTGATGAAGGGGCAAAAATTGACCGAGGTATAAGATTC | |
| | CTCCATGATCAGATAATGAGTGTGAAAACAGTGGATCTC | |
| | ACACTGGTGATTTATGGATCGTTCAGACATTGGGGTCAT | |
| | CCTTTTATAGATTATTACACTGGACTAGAAAAATTACAT | |
| | TCCCAAGTAACCATGAAGAAAGATATTGATGTGTCATAT | |
| | GCAAAAGCACTTGCAAGTGATTTAGCTCGGATTGTTCTA | |
| | TTTCAACAGTTCAATGATCATAAAAAGTGGTTCGTGAAT | |
| | GGAGACTTGCTCCCTCATGATCATCCCTTTAAAAGTCAT | |
| | GTTAAAGAAAATACATGGCCCACAGCTGCTCAAGTTCAA | |
| | GATTTTGGAGATAAATGGCATGAACTTCCGCTGATTAAA | |
| | TGTTTTGAAATACCCGACTTACTAGACCCATCGATAATA | |
| | TACTCTGACAAAAGTCATTCAATGAATAGGTCAGAGGTG | |
| | TTGAAACATGTCCGAATGAATCCGAACACTCCTATCCCT | |
| | AGTAAAAAGGTGTTGCAGACTATGTTGGACACAAAGGCT | |
| | ACCAATTGGAAAGAATTTCTTAAAGAGATTGATGAGAAG | |
| | GGCTTAGATGATGATGATCTAATTATTGGTCTTAAAGGA | |
| | AAGGAGAGGGAACTGAAGTTGGCAGGTAGATTTTTCTCC | |
| | CTAATGTCTTGGAAATTGCGAGAATACTTTGTAATTACC | |
| | GAATATTTGATAAAGACTCATTTCGTCCCTATGTTTAAA | |
| | GGCCTGACAATGGCGGACGATCTAACTGCAGTCATTAAA | |
| | AAGATGTTAGATTCCTCATCCGGCCAAGGATTGAAGTCA | |
| | TATGAGGCAATTTGCATAGCCAATCACATTGATTACGAA | |
| | AAATGGAATAACCACCAAAGGAAGTTATCAAACGGCCCA | |
| | GTGTTCCGAGTTATGGGCCAGTTCTTAGGTTATCCATCC | |
| | TTAATCGAGAGAACTCATGAATTTTTTGAGAAAAGTCTT | |
| | ATATACTACAATGGAAGACCAGACTTGATGCGTGTTCAC | |
| | AACAACACACTGATCAATTCAACCTCCCAACGAGTTTGT | |
| | TGGCAAGGACAAGAGGGTGGACTGGAAGGTCTACGGCAA | |
| | AAAGGATGGAGTATCCTCAATCTACTGGTTATTCAAAGA | |
| | GAGGCTAAAATCAGAAACACTGCTGTCAAAGTCTTGGCA | |
| | CAAGGTGATAATCAAGTTATTTGCACACAGTATAAAACG | |
| | AAGAAATCGAGAAACGTTGTAGAATTACAGGGTGCTCTC | |
| | AATCAAATGGTTTCTAATAATGAGAAAATTATGACTGCA | |
| | ATCAAAATAGGGACAGGGAAGTTAGGACTTTTGATAAAT | |
| | GACGATGAGACTATGCAATCTGCAGATTACTTGAATTAT | |
| | GGAAAAATACCGATTTTCCGTGGAGTGATTAGAGGGTTA | |
| | GAGACCAAGAGATGGTCACGAGTGACTTGTGTCACCAAT | |
| | GACCAAATACCCACTTGTGCTAATATAATGAGCTCAGTT | |
| | TCCACAAATGCTCTCACCGTAGCTCATTTTGCTGAGAAC | |
| | CCAATCAATGCCATGATACAGTACAATTATTTTGGGACA | |
| | TTTGCTAGACTCTTGTTGATGATGCATGATCCTGCTCTT | |
| | CGTCAATCATTGTATGAAGTTCAAGATAAGATACCGGGC | |
| | TTGCACAGTTCTACTTTCAAATACGCCATGTTGTATTTG | |
| | GACCCTTCCATTGGAGGAGTGTCGGGCATGTCTTTGTCC | |
| | AGGTTTTTGATTAGAGCCTTCCCAGATCCCGTAACAGAA | |
| | AGTCTCTCATTCTGGAGATTCATCCATGTACATGCTCGA | |
| | AGTGAGCATCTGAAGGAGATGAGTGCAGTATTTGGAAAC | |
| | CCCGAGATAGCCAAGTTTCGAATAACTCACATAGACAAG | |
| | CTAGTAGAAGATCCAACCTCTCTGAACATCGCTATGGGA | |
| | ATGAGTCCAGCGAACTTGTTAAAGACTGAGGTTAAAAAA | |
| | TGCTTAATCGAATCAAGACAAACCATCAGGAACCAGGTG | |
| | ATTAAGGATGCAACCATATATTTGTATCATGAAGAGGAT | |
| | CGGCTCAGAAGTTTCTTATGGTCAATAAATCCTCTGTTC | |
| | CCTAGATTTTTAAGTGAATTCAAATCAGGCACTTTTTTG | |
| | GGAGTCGCAGACGGGCTCATCAGTCTATTTCAAAATTCT | |
| | CGTACTATTCGGAACTCCTTTAAGAAAAAGTATCATAGG | |
| | GAATTGGATGATTTGATTGTGAGGAGTGAGGTATCCTCT | |
| | TTGACACATTTAGGGAAACTTCATTTGAGAAGGGGATCA | |
| | TGTAAAATGTGGACATGTTCAGCTACTCATGCTGACACA | |
| | TTAAGATACAAATCCTGGGGCCGTACAGTTATTGGGACA | |
| | ACTGTACCCCATCCATTAGAAATGTTGGGTCCACAACAT | |
| | CGAAAAGAGACTCCTTGTGCACCATGTAACACATCAGGG | |
| | TTCAATTATGTTTCTGTGCATTGTCCAGACGGGATCCAT | |
| | GACGTCTTTAGTTCACGGGGACCATTGCCTGCTTATCTA | |
| | GGGTCTAAAACATCTGAATCTACATCTATTTTGCAGCCT | |
| | TGGGAAAGGGAAAGCAAAGTCCCACTGATTAAAAGAGCT | |
| | ACACGTCTTAGAGATGCTATCTCTTGGTTTGTTGAACCC | |
| | GACTCTAAACTAGCAATGACTATACTTTCTAACATCCAC | |
| | TCTTTAACAGGCGAAGAATGGACCAAAAGGCAGCATGGG | |
| | TTCAAAAGAACAGGGTCTGCCCTTCATAGGTTTTCGACA | |
| | TCTCGGATGAGCCATGGTGGGTTCGCATCTCAGAGCACT | |
| | GCAGCATTGACCAGGTTGATGGCAACTACAGACACCATG | |
| | AGGGATCTGGGAGATCAGAATTTCGACTTTTTATTCCAA | |
| | GCAACGTTGCTCTATGCTCAAATTACCACCACTGTTGCA | |
| | AGAGACGGATGGATCACCAGTTGTACAGATCATTATCAT | |
| | ATTGCCTGTAAGTCCTGTTTGAGACCCATAGAAGAGATC | |
| | ACCCTGGACTCAAGTATGGACTACACGCCCCCAGATGTA | |
| | TCCCATGTGCTGAAGACATGGAGGAATGGGGAAGGTTCG | |
| | TGGGGACAAGAGATAAAACAGATCTATCCTTTAGAAGGG | |
| | AATTGGAAGAATTTAGCACCTGCTGAGCAATCCTATCAA | |
| | GTCGGCAGATGTATAGGTTTTCTATATGGAGACTTGGCG | |
| | TATAGAAAATCTACTCATGCCGAGGACAGTTCTCTATTT | |
| | CCTCTATCTATACAAGGTCGTATTAGAGGTCGAGGTTTC | |
| | TTAAAAGGGTTGCTAGACGGATTAATGAGAGCAAGTTGC | |
| | TGCCAAGTAATACACCGGAGAAGTCTGGCTCATTTGAAG | |
| | AGGCCGGCCAACGCAGTGTACGGAGGTTTGATTTACTTG | |
| | ATTGATAAATTGAGTGTATCACCTCCATTCCTTTCTCTT | |
| | ACTAGATCAGGACCTATTAGAGACGAATTAGAAACGATT | |
| | CCCCACAAGATCCCAACCTCCTATCCGACAAGCAACCGT | |
| | GATATGGGGGTGATTGTCAGAAATTACTTCAAATACCAA | |
| | TGCCGTCTAATTGAAAAGGGAAAATACAGATCACATTAT | |
| | TCACAATTATGGTTATTCTCAGATGTCTTATCCATAGAC | |
| | TTCATTGGACCATTCTCTATTTCCACCACCCTCTTGCAA | |
| | ATCCTATACAAGCCATTTTTATCTGGGAAAGATAAGAAT | |
| | GAGTTGAGAGAGCTGGCAAATCTTTCTTCATTGCTAAGA | |
| | TCAGGAGAGGGGTGGGAAGACATACATGTGAAATTCTTC | |
| | ACCAAGGACATATTATTGTGTCCAGAGGAAATCAGACAT | |
| | GCTTGCAAGTTCGGGATTGCTAAGGATAATAATAAAGAC | |
| | ATGAGCTATCCCCCTTGGGGAAGGGAATCCAGAGGGACA | |
| | ATTACAACAATCCCTGTTTATTATACGACCACCCCTTAC | |
| | CCAAAGATGCTAGAGATGCCTCCAAGAATCCAAAATCCC | |
| | CTGCTGTCCGGAATCAGGTTGGGCCAATTACCAACTGGC | |
| | GCTCATTATAAAATTCGGAGTATATTACATGGAATGGGA | |
| | ATCCATTACAGGGACTTCTTGAGTTGTGGAGACGGCTCC | |
| | GGAGGGATGACTGCTGCATTACTACGAGAAAATGTGCAT | |
| | AGCAGAGGAATATTCAATAGTCTGTTAGAATTATCAGGG | |
| | TCAGTCATGCGAGGCGCCTCTCCTGAGCCCCCCAGTGCC | |
| | CTAGAAACTTTAGGAGGAGATAAATCGAGATGTGTAAAT | |
| | GGTGAAACATGTTGGGAATATCCATCTGACTTATGTGAC | |
| | CCAAGGACTTGGGACTATTTCCTCCGACTCAAAGCAGGC | |
| | TTGGGGCTTCAAATTGATTTAATTGTAATGGATATGGAA | |
| | GTTCGGGATTCTTCTACTAGCCTGAAAATTGAGACGAAT | |
| | GTTAGAAATTATGTGCACCGGATTTTGGATGAGCAAGGA | |
| | GTTTTAATCTACAAGACTTATGGAACATATATTTGTGAG | |
| | AGCGAAAAGAATGCAGTAACAATCCTTGGTCCCATGTTC | |
| | AAGACGGTCGACTTAGTTCAAACAGAATTTAGTAGTTCT | |
| | CAAACGTCTGAAGTATATATGGTATGTAAAGGTTTGAAG | |
| | AAATTAATCGATGAACCCAATCCCGATTGGTCTTCCATC | |
| | AATGAATCCTGGAAAAACCTGTACGCATTCCAGTCATCA | |
| | GAACAGGAATTTGCCAGAGCAAAGAAGGTTAGTACATAC | |
| | TTTACCTTGACAGGTATTCCCTCCCAATTCATTCCTGAT | |
| | CCTTTTGTAAACATTGAGACTATGCTACAAATATTCGGA | |
| | GTACCCACGGGTGTGTCTCATGCGGCTGCCTTAAAATCA | |
| | TCTGATAGACCTGCAGATTTATTGACCATTAGCCTTTTT | |
| | TATATGGCGATTATATCGTATTATAACATCAATCATATC | |
| | AGAGTAGGACCGATACCTCCGAACCCCCCATCAGATGGA | |
| | ATTGCACAAAATGTGGGGATCGCTATAACTGGTATAAGC | |
| | TTTTGGCTGAGTTTGATGGAGAAAGACATTCCACTATAT | |
| | CAACAGTGTTTAGCAGTTATCCAGCAATCATTCCCGATT | |
| | AGGTGGGAGGCTGTTTCAGTAAAAGGAGGATACAAGCAG | |
| | AAGTGGAGTACTAGAGGTGATGGGCTCCCAAAAGATACC | |
| | CGAATTTCAGACTCCTTGGCCCCAATCGGGAACTGGATC | |
| | AGATCTCTGGAATTGGTCCGAAACCAAGTTCGTCTAAAT | |
| | CCATTCAATGAGATCTTGTTCAATCAGCTATGTCGTACA | |
| | GTGGATAATCATTTGAAATGGTCAAATTTGCGAAGAAAC | |
| | ACAGGAATGATTGAATGGATCAATAGACGAATTTCAAAA | |
| | GAAGACCGGTCTATACTGATGTTGAAGAGTGACCTACAC | |
| | GAGGAAAACTCTTGGAGAGATTAAAAAATCATGAGGAGA | |
| | CTCCAAACTTTAAGTATGAAAAAAACTTTGATCCTTAAG | |
| | ACCCTCTTGTGGTTTTTATTTTTTATCTGGTTTTGTGGT | |
| | CTTCGT | |
| SEQ ID NO: 50 | NRTLTLFNVTRNDARAYVSGIQNSVSANRSDPVTLDVLP | multi- |
| | DSSYLSGANLNLSCHSASPQYSWRINGIPQQHTQVLFIA | antigenic |
| | KITPNNNGTYACFVSNLATGRNNSIVKSITVSASGTSPG | domain |
| | LSATEYKLVVVGADGVGKSALTIQLIQ | ATP132 |
| SEQ ID NO: 51 | MKRYKNRVASRKSRAKFKQLLQHYREVAAAKSSENDRLR | ATP132 |
| | LLLKNRTLTLFNVTRNDARAYVSGIQNSVSANRSDPVTL | |
| | DVLPDSSYLSGANLNLSCHSASPQYSWRINGIPQQHTQV | |
| | LFIAKITPNNNGTYACFVSNLATGRNNSIVKSITVSASG | |
| | TSPGLSATEYKLVVVGADGVGKSALTIQLIQSTVHEILS | |
| | KLSLEGDHSTPPSAYGSVKPYTNFDAE | |

1. A composition comprising:
(i) at least one tumor antigen, or a fragment or sequence variant thereof,
wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2);
(ii) a nucleic acid encoding said tumor antigen;
(iii) an antigen-presenting cell (APC) containing (i) or (ii); or
(iv) a T-cell expressing either a T-cell receptor or a CAR T-cell receptor targeting said tumor antigen.

2. The composition according to claim 1, wherein the composition further comprises at least one tumor antigen selected from the group consisting of CEACAM5, DUOXA2, and KRAS, or a fragment or sequence variant thereof.

3. The composition according to claim 2, wherein KRAS, or the fragment thereof, is KRAS-G12D, or a fragment thereof, or KRAS-G12V, or a fragment thereof.

4. The composition according to claim 2, wherein the tumor antigen selected from CEACAM5, DUOXA2, KRAS, KRAS-uORF1, TPX2-uORF1 and AURKA-uORF2, or the fragment or sequence variant thereof, comprises a CD4+ and/or a CD8+ epitope.

5. The composition according to claim 1, wherein the tumor antigen comprises at least one of amino acid, wherein the amino acid sequence is
an amino acid sequence according to SEQ ID NO: 9, or a sequence variant thereof having at least 70% sequence identity;
an amino acid sequence according to SEQ ID NO: 11, or a sequence variant thereof having at least 70% sequence identity;
an amino acid sequence according to SEQ ID NO: 12, or a sequence variant thereof having at least 70% sequence identity;
an amino acid sequence according to SEQ ID NO: 1 or 2, or a sequence variant thereof having at least 70% sequence identity;
an amino acid sequence according to SEQ ID NO: 3 or 4, or a sequence variant thereof having at least 70% sequence identity;
an amino acid sequence according to SEQ ID NO: 5, or a sequence variant thereof having at least 70% sequence identity; or
an amino acid sequence according to SEQ ID NO: 10, or a sequence variant thereof having at least 70% sequence identity.

6. A peptide comprising:
a) a cell penetrating peptide;
b) a multi-antigenic domain comprising at least one tumor antigen, or a fragment or sequence variant thereof,
wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); and
c) a TLR peptide agonist.

7. The peptide according to claim 6, wherein the multi-antigenic domain further comprises at least one tumor antigen selected from the group consisting of CEACAM5 or a fragment thereof, DUOXA2 or a fragment thereof, and KRAS or a fragment thereof, wherein each fragment has a minimum length of 8 amino acids.

8. The peptide according to claim 6, wherein the multi-antigenic domain comprises three or more different antigens, or fragments thereof having a minimum length of 8 amino acids, in particular 3, 4, 5, 6, 7, 8, 9, 10 or more different antigens or fragments thereof.

9. The peptide according to claim 6, wherein the multi-antigenic domain comprises;
a tumor antigen, which is encoded in a 5′-upstream open reading frame (uORF) within the 5′ UTR of the KRAS mRNA (KRAS-uORF1),
a tumor antigen, which is encoded in a 5′-upstream open reading frame (uORF) within the 5′ UTR of the TPX2 mRNA (TPX2-uORF1), and
a tumor antigen, which is encoded in a 5′-upstream open reading frame (uORF) within the 5′ UTR of the AURKA mRNA (AURKA-uORF2).

10. The peptide according to claim 6, wherein the multi-antigenic domain comprises, preferably in N- to C-terminal direction:
CEACAM5 or a fragment thereof having a minimum length of 8 amino acids,
KRAS or a fragment thereof having a minimum length of 8 amino acids,
KRAS-uORF1, or a fragment thereof having a minimum length of 8 amino acids,
TPX2-uORF1, or a fragment thereof having a minimum length of 8 amino acids,
AURKA-uORF2, or a fragment thereof having a minimum length of 8 amino acids, and
DUOXA2 or a fragment thereof having a minimum length of 8 amino acids.

11. The peptide according to claim 7, wherein KRAS, or the fragment thereof, is KRAS-G12D, or a fragment thereof, or KRAS-G12V, or a fragment thereof.

12. The peptide according to claim 7, wherein the fragment of CEACAM5 comprises an amino acid sequence according to any one of SEQ ID NOs 6-8, or a sequence variant thereof having at least 70% sequence identity, preferably wherein the multi-antigenic domain comprises an amino acid sequence according to SEQ ID NO: 9, or a sequence variant thereof having at least 70% sequence identity.

13. The peptide according to claim 6, wherein the multi-antigenic domain comprises at least one amino acid sequence selected from the group consisting of:
an amino acid sequence according to SEQ ID NO: 9, or a sequence variant thereof having at least 70% sequence identity;
an amino acid sequence according to SEQ ID NO: 12, or a sequence variant thereof having at least 70% sequence identity;
an amino acid sequence according to SEQ ID NO: 11, or a sequence variant thereof having at least 70% sequence identity;
an amino acid sequence according to SEQ ID NO: 1 or 2, or a sequence variant thereof having at least 70% sequence identity;
an amino acid sequence according to SEQ ID NO: 3 or 4, or a sequence variant thereof having at least 70% sequence identity;
an amino acid sequence according to SEQ ID NO: 5, or a sequence variant thereof having at least 70% sequence identity; and
an amino acid sequence according to SEQ ID NO: 10, or a sequence variant thereof having at least 70% sequence identity.

14. The peptide according to claim 6, wherein the multi-antigenic domain comprises, preferably in N- to C-terminal direction:
an amino acid sequence according to SEQ ID NO: 9, or a sequence variant thereof having at least 70% sequence identity;
an amino acid sequence according to SEQ ID NO: 11, or a sequence variant thereof having at least 70% sequence identity, and/or an amino acid sequence according to SEQ ID NO: 12, or a sequence variant thereof having at least 70% sequence identity;
an amino acid sequence according to SEQ ID NO: 1 or 2, or a sequence variant thereof having at least 70% sequence identity;
an amino acid sequence according to SEQ ID NO: 3 or 4, or a sequence variant thereof having at least 70% sequence identity;
an amino acid sequence according to SEQ ID NO: 5, or a sequence variant thereof having at least 70% sequence identity; and
an amino acid sequence according to SEQ ID NO: 10, or a sequence variant thereof having at least 70% sequence identity.

15. The peptide according to claim 6, wherein the multi-antigenic domain comprises an amino acid sequence according to SEQ ID NO: 13 or 14, or a sequence variant thereof having at least 70% sequence identity.

16. The peptide according to claim 6, wherein the cell penetrating peptide has an amino acid sequence comprising or consisting of an amino acid sequence according to SEQ ID NO: 15, SEQ ID NO: 21, SEQ ID NO: 22, or SEQ ID NO: 23, or a sequence variants thereof having at least 70% sequence identity.

17. The peptide according to claim 6, wherein the cell penetrating peptide has an amino acid sequence comprising or consisting of an amino acid sequence according to SEQ ID NO: 15 or a sequence variant, thereof having at least 70% sequence identity.

18. The peptide according to claim 6, wherein the TLR peptide agonist is a TLR2 peptide agonist and/or a TLR4 peptide agonist.

19. The peptide according to claim 6, wherein the TLR peptide agonist comprises or consists of an amino acid sequence according to SEQ ID NO: 16 or 24; or a sequence variant thereof having at least 70% sequence identity.

20. The peptide according to claim 6, wherein
a) the cell penetrating peptide has an amino acid sequence comprising or consisting of an amino acid sequence according to SEQ ID NO: 15, SEQ ID NO: 21, SEQ ID NO: 22 or SEQ ID NO: 23, or a sequence variants thereof having at least 70% sequence identity;
b) the multi-antigenic domain comprises
a fragment of CEACAM5 having a minimum length of 8 amino acids,
a fragment of DUOXA2 having a minimum length of 8 amino acids,
a fragment of KRAS having a minimum length of 8 amino acids,
KRAS-uORF1, or a fragment thereof having a minimum length of 8 amino acids,
TPX2-uORF1, or a fragment thereof having a minimum length of 8 amino acids, and/or
AURKA-uORF2, or a fragment thereof having a minimum length of 8 amino acids; and
c) the TLR peptide agonist is a TLR2 peptide agonist and/or a TLR4 peptide agonist.

21. The peptide according to claim 6, wherein the peptide comprises:
a) a cell penetrating peptide having an amino acid sequence comprising or consisting of an amino acid sequence according to SEQ ID NO: 15, or a sequence variant, thereof having at least 70% sequence identity;
b) a multi-antigenic domain comprising
an amino acid sequence according to SEQ ID NO: 9, or a sequence variant thereof having at least 70% sequence identity;
an amino acid sequence according to SEQ ID NO: 11, or a sequence variant thereof having at least 70% sequence identity, and/or an amino acid sequence according to SEQ ID NO: 12, or a sequence variant thereof having at least 70% sequence identity;
an amino acid sequence according to SEQ ID NO: 1, or a sequence variant thereof having at least 70% sequence identity;
an amino acid sequence according to SEQ ID NO: 3, or a sequence variant thereof having at least 70% sequence identity;
an amino acid sequence according to SEQ ID NO: 5, or a sequence variant thereof having at least 70% sequence identity; and
an amino acid sequence according to SEQ ID NO: 10, or a sequence variant thereof having at least 70% sequence identity; and
c) a TLR peptide agonist having an amino acid sequence comprising or consisting of an amino acid sequence according to SEQ ID NO: 16, or a sequence variants thereof having at least 70% sequence identity.

22. The peptide according to claim 6, wherein the peptide comprises in N-terminal to C-terminal direction:
a) a cell penetrating peptide having an amino acid sequence comprising or consisting of an amino acid sequence according to SEQ ID NO: 15, or a sequence variants thereof having at least 90% sequence identity;
b) a multi-antigenic domain comprising
an amino acid sequence according to SEQ ID NO: 9, or a sequence variant thereof having at least 90% sequence identity;
an amino acid sequence according to SEQ ID NO: 11, or a sequence variant thereof having at least 90% sequence identity, and/or an amino acid sequence according to SEQ ID NO: 12, or a sequence variant thereof having at least 90%% sequence identity;
an amino acid sequence according to SEQ ID NO: 1, or a sequence variant thereof having at least 90% sequence identity;
an amino acid sequence according to SEQ ID NO: 3, or a sequence variant thereof having at least 90% sequence identity;
an amino acid sequence according to SEQ ID NO: 5, or a sequence variant thereof having at least 90% sequence identity; and
an amino acid sequence according to SEQ ID NO: 10, or a sequence variant thereof having at least 90% sequence identity; and
c) a TLR peptide agonist having an amino acid sequence comprising or consisting of an amino acid sequence according to SEQ ID NO: 16, or a sequence variants thereof having at least 90% sequence identity.

23. The peptide according to claim 6, wherein the peptide comprises or consists of an amino acid sequence according to SEQ ID NO: 17, SEQ ID NO: 18, SEQ ID NO: 46, or SEQ ID NO: 47, or a sequence variant thereof having at least 70% sequence identity.

24. A recombinant vesicular stomatitis virus (VSV) encoding a multi-antigenic domain comprising at least one tumor antigen, or fragment or sequence variant thereof, wherein the tumor antigen is encoded in a 5′-upstream open reading frame (uORF) within the 5′ UTR of 4W-KRAS mRNA (KRAS-uORF), of TPX2 mRNA (TPX2-uORF) or of 4*e-AURKA mRNA (AURKA-uORF).

25. The vesicular stomatitis virus (VSV) according to claim 24, wherein the multi-antigenic domain further comprises at least one tumor antigen selected from the group consisting of CEACAM5 or a fragment thereof, DUOXA2 or a fragment thereof, and KRAS or a fragment thereof, wherein each fragment has a minimum length of 8 amino acids.

26. The vesicular stomatitis virus (VSV) according to claim 24, wherein the multi-antigenic domain comprises three or more different antigens, or fragments thereof having a minimum length of 8 amino acids.

27. The vesicular stomatitis virus (VSV) according to claim 24, wherein the multi-antigenic domain comprises:
a tumor antigen, which is encoded in a 5′-upstream open reading frame (uORF) within the 5′ UTR of the KRAS mRNA (KRAS-uORF1),
a tumor antigen, which is encoded in a 5′-upstream open reading frame (uORF) within the 5′ UTR of the TPX2 mRNA (TPX2-uORF1), and
a tumor antigen, which is encoded in a 5′-upstream open reading frame (uORF) within the 5′ UTR of the AURKA mRNA (AURKA-uORF2).

28. The vesicular stomatitis virus (VSV) according to claim 24, wherein the multi-antigenic domain comprises:
CEACAM5 or a fragment thereof having a minimum length of 8 amino acids,
KRAS or a fragment thereof having a minimum length of 8 amino acids,
KRAS-uORF1, or a fragment thereof having a minimum length of 8 amino acids,
TPX2-uORF1, or a fragment thereof having a minimum length of 8 amino acids,
AURKA-uORF2, or a fragment thereof having a minimum length of 8 amino acids, and
DUOXA2 or a fragment thereof having a minimum length of 8 amino acids.

29. The vesicular stomatitis virus (VSV) according to claim 24, wherein KRAS, or the fragment thereof, is KRAS-G12D, or a fragment thereof, or KRAS-G12V, or a fragment thereof.

30. The vesicular stomatitis virus (VSV) according to claim 24, wherein the fragment of CEACAM5 comprises an amino acid sequence according to any one of SEQ ID NOs 6-8, or a sequence variant thereof having at least 70% sequence identity.

31. The vesicular stomatitis virus (VSV) according to claim 24, wherein the multi-antigenic domain comprises at least one of amino acid sequence selected from the group consisting of:
an amino acid sequence according to SEQ ID NO: 9, or a sequence variant thereof having at least 70% sequence identity;
an amino acid sequence according to SEQ ID NO: 11, or a sequence variant thereof having at least 70% sequence identity;
an amino acid sequence according to SEQ ID NO: 12, or a sequence variant thereof having at least 70% sequence identity;
an amino acid sequence according to SEQ ID NO: 1 or 2, or a sequence variant thereof having at least 70% sequence identity;
an amino acid sequence according to SEQ ID NO: 3 or 4, or a sequence variant thereof having at least 70% sequence identity;
an amino acid sequence according to SEQ ID NO: 5, or a sequence variant thereof having at least 70% sequence identity; and
an amino acid sequence according to SEQ ID NO: 10, or a sequence variant thereof having at least 70% sequence identity.

32. The vesicular stomatitis virus (VSV) according to claim 24, wherein the multi-antigenic domain comprises:
an amino acid sequence according to SEQ ID NO: 9, or a sequence variant thereof having at least 70% sequence identity;
an amino acid sequence according to SEQ ID NO: 11, or a sequence variant thereof having at least 70% sequence identity, and/or an amino acid sequence according to SEQ ID NO: 12, and/or a sequence variant thereof having at least 70% sequence identity;
an amino acid sequence according to SEQ ID NO: 1, or a sequence variant thereof having at least 70% sequence identity;
an amino acid sequence according to SEQ ID NO: 3, or a sequence variant thereof having at least 70% sequence identity;
an amino acid sequence according to SEQ ID NO: 5, or a sequence variant thereof having at least 70% sequence identity; and
an amino acid sequence according to SEQ ID NO: 10, or a sequence variant thereof having at least 70% sequence identity.

33. The vesicular stomatitis virus (VSV) according to claim 24, wherein the multi-antigenic domain comprises in N- to C-terminal direction:
an amino acid sequence according to SEQ ID NO: 9, or a sequence variant thereof having at least 90% sequence identity;
an amino acid sequence according to SEQ ID NO: 11, or a sequence variant thereof having at least 90% sequence identity, and/or an amino acid sequence according to SEQ ID NO: 12, and/or a sequence variant thereof having at least 90%-sequence identity;
an amino acid sequence according to SEQ ID NO: 1, or a sequence variant thereof having at least 90% sequence identity;
an amino acid sequence according to SEQ ID NO: 3, or a sequence variant thereof having at least 90% sequence identity;
an amino acid sequence according to SEQ ID NO: 5, or a sequence variant thereof having at least 90% sequence identity; and
an amino acid sequence according to SEQ ID NO: 10, or a sequence variant thereof having at least 90% sequence identity.

34. The vesicular stomatitis virus (VSV) according to claim 24, wherein the multi-antigenic domain comprises an amino acid sequence according to SEQ ID NO: 19 or SEQ ID NO: 48, or a sequence variant thereof having at least 70% sequence identity.

35. The vesicular stomatitis virus (VSV) according to claim 24, wherein the vesicular stomatitis virus (VSV) is an oncolytic vesicular stomatitis virus (VSV).

36. The vesicular stomatitis virus (VSV) according to claim 24, wherein the vesicular stomatitis virus (VSV) is replication-competent.

37. The vesicular stomatitis virus (VSV) according to claim 24, wherein the gene coding for the glycoprotein G is replaced by the gene coding for the glycoprotein GP of Lymphocyte choriomeningitis virus (LCMV).

38. The vesicular stomatitis virus (VSV) according to claim 24, wherein
it encodes in its genome a multi-antigenic domain comprising at least one tumor antigen, or fragment or sequence variant thereof, wherein the tumor antigen is encoded in a 5′-upstream open reading frame (uORF) within the 5′ UTR of the KRAS mRNA (KRAS-uORF), of the TPX2 mRNA (TPX2-uORF) or of the AURKA mRNA (AURKA-uORF), and wherein the multi-antigenic domain further comprises at least one tumor antigen selected from the group consisting of CEACAM5 or a fragment thereof, DUOXA2 or a fragment thereof, and KRAS or a fragment thereof, wherein each fragment has a minimum length of 8 amino acids,
it encodes in its genome a vesicular stomatitis virus nucleoprotein (N), large protein (L), phosphoprotein (P), and matrix protein (M),
the gene coding for the glycoprotein G of the vesicular stomatitis virus is replaced by the gene coding for the glycoprotein GP of lymphocyte choriomeningitis virus (LCMV), and/or
the glycoprotein G of the vesicular stomatitis virus is replaced by the glycoprotein GP of LCMV.

39. The vesicular stomatitis virus (VSV) according to claim 24, wherein
it encodes in its genome a vesicular stomatitis virus nucleoprotein (N) comprising an amino acid as set forth in SEQ ID NO: 26, or a sequence variant thereof having at least 80%, 85%, 90%, 92%, 94%, 96%, 98% sequence identity;
it encodes in its genome a vesicular stomatitis virus phosphoprotein (P) comprising an amino acid as set forth in SEQ ID NO: 27, or a sequence variant thereof having at least 80%, 85%, 90%, 92%, 94%, 96%, 98% sequence identity;
it encodes in its genome a vesicular stomatitis virus large protein (L) comprising an amino acid as set forth in SEQ ID NO: 28, or a sequence variant thereof having at least 80%, 85%, 90%, 92%, 94%, 96%, 98% sequence identity;
it encodes in its genome a vesicular stomatitis virus matrix protein (M) comprising an amino acid as set forth in SEQ ID NO: 29, or a sequence variant thereof having at least 80%, 85%, 90%, 92%, 94%, 96%, 98% sequence identity;
it encodes in its genome a vesicular stomatitis virus comprising a multi-antigenic domain comprising at least one tumor antigen, or fragment or sequence variant thereof, wherein the tumor antigen is encoded in a 5′-upstream open reading frame (uORF) within the 5′ UTR of the KRAS mRNA (KRAS-uORF), of the TPX2 mRNA (TPX2-uORF) or of the AURKA mRNA (AURKA-uORF), and wherein the multi-antigenic domain further comprises at least one tumor antigen selected from the group consisting of CEACAM5 or a fragment thereof, DUOXA2 or a fragment thereof, and KRAS or a fragment thereof, wherein each fragment has a minimum length of 8 amino acids,
the gene coding for the glycoprotein G of the vesicular stomatitis virus is replaced by the gene coding for the glycoprotein GP of lymphocyte choriomeningitis virus (LCMV), and/or
the glycoprotein G is replaced by the glycoprotein GP of LCMV.

40. The vesicular stomatitis virus (VSV) according to claim 24, and wherein it encodes in its genome
a phosphoprotein (P) comprising the amino acid sequence according to SEQ ID NO: 27, or a sequence variant thereof having at least 70% sequence identity,
a nucleoprotein (N) comprising the amino acid sequence according to SEQ ID NO: 26, or a sequence variant thereof having at least 70% sequence identity,
a matrix protein (M) comprising the amino acid sequence according to SEQ ID NO: 29, or a sequence variant thereof having at least 70% sequence identity,
a large protein (L) comprising the amino acid sequence according to SEQ ID NO: 28, or a sequence variant thereof having at least 70% sequence identity,
a glycoprotein (GP) comprising the amino acid sequence according to SEQ ID NO: 25, or a sequence variant thereof having at least 70% sequence identity, and
a multi-antigenic domain which comprises the amino acid sequence according to SEQ ID NO: 19 or SEQ ID NO: 48, or a sequence variant thereof having at least 70% sequence identity.

41. The vesicular stomatitis virus (VSV) according to claim 24, wherein its RNA genome comprises or consists of an RNA sequence according to SEQ ID NO: 30, or a sequence variant thereof having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity.

42. A vaccine comprising:
(i) a composition comprising:
a) at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2);
b) a nucleic acid encoding said tumor antigen;
c) an antigen-presenting cell (APC) containing (a) or (b); or
d) a T-cell expressing either a T-cell receptor or a CAR T-cell receptor targeting said tumor antigen;
(ii) a peptide comprising:
a) a cell penetrating peptide;
b) a multi-antigenic domain comprising at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); and
c) a TLR peptide agonist;
(iii) a vesicular stomatitis virus (VSV) encoding a multi-antigenic domain comprising at least one tumor antigen, or fragment or sequence variant thereof, wherein the tumor antigen is encoded in a 5′-upstream open reading frame (uORF) within the 5′ UTR of KRAS mRNA (KRAS-uORF), of TPX2 mRNA (TPX2-uORF) or of AURKA mRNA (AURKA-uORF).

43. The vaccine according to claim 42, wherein the vaccine comprises
(i) the peptide; and
(ii) the vesicular stomatitis virus (VSV).

44. (canceled)

45. (canceled)

46. The vaccine according to claim 43, wherein the multi-antigenic domain encoded in the genome of the vesicular stomatitis virus (VSV) comprises an antigen, or a fragment or sequence variant thereof, which is contained in the multi-antigenic domain of the peptide.

47. The vaccine according to claim 46, wherein the multi-antigenic domain encoded in the genome of the vesicular stomatitis virus (VSV) comprises the amino acid sequences of each of the antigens, or a fragments or sequence variant thereof, which are contained in the multi-antigenic domain of the peptide.

48. The vaccine according to claim 43, wherein the multi-antigenic domain of the peptide comprises an antigen, or a fragment or sequence variant thereof, which is contained in the multi-antigenic domain encoded in the genome of the vesicular stomatitis virus (VSV).

49. The vaccine according to claim 48, wherein the multi-antigenic domain of the peptide comprises the amino acid sequences of each of the antigens, or a fragment or a sequence variant thereof, of the multi-antigenic domain which is encoded in the genome of the vesicular stomatitis virus (VSV).

50. The vaccine according to claim 43, wherein
the multi-antigenic domain of the peptide comprises an amino acid sequence according to SEQ ID NO: 13 or 14, or a sequence variant thereof having at least 70% sequence identity, and
the multi-antigenic domain encoded in the genome of the vesicular stomatitis virus (VSV) comprises the amino acid sequence according to SEQ ID NO: 19 or SEQ ID NO: 48, or a sequence variant thereof having at least 70% sequence identity.

51. The vaccine according to claim 43, wherein
the peptide comprises an amino acid sequence according to SEQ ID NO: 17, SEQ ID NO: 18, SEQ ID NO: 46, or SEQ ID NO: 47, or a sequence variant thereof having at least 70% sequence identity, and
the vesicular stomatitis virus (VSV) comprises an RNA genome comprising an RNA sequence according to SEQ ID NO: 30, or a sequence variant thereof having at least 70% sequence identity.

52. The vaccine according to claim 43, further comprising
an inhibitor of the PD-1/PD-L1 pathway.

53. The vaccine according to claim 52, wherein the inhibitor of the PD-1/PD-L1 pathway is selected from the group consisting of pembrolizumab; nivolumab; pidilizumab; cemiplimab; PDR-001; atezolizumab; avelumab; durvalumab, ezabenlimab, an antibody comprising a heavy chain comprising the amino acid sequence of SEQ ID NO: 31 and a light chain comprising the amino acid sequence of SEQ ID NO: 32; an antibody comprising a heavy chain comprising the amino acid sequence of SEQ ID NO: 33 and a light chain comprising the amino acid sequence of SEQ ID NO: 34; and an antibody comprising a heavy chain comprising the amino acid sequence of SEQ ID NO: 35 and a light chain comprising the amino acid sequence of SEQ ID NO: 36.

54.-71. (canceled)

72. A kit comprising
(i) a peptide comprising
a) a cell penetrating peptide;
b) a multi-antigenic domain comprising at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); and
c) a TLR peptide agonist; and
(ii) a vesicular stomatitis virus (VSV) encoding a multi-antigenic domain comprising at least one tumor antigen, or fragment or sequence variant thereof, wherein the tumor antigen is encoded in a 5′-upstream open reading frame (uORF) within the 5′ UTR of KRAS mRNA (KRAS-uORF), of TPX2 mRNA (TPX2-uORF) or of AURKA mRNA (AURKA-uORF).

73. The kit according to claim 72, wherein the multi-antigenic domain encoded in the genome of the vesicular stomatitis virus (VSV) comprises an antigen, or a fragment or sequence variant thereof, which is contained in the multi-antigenic domain of the peptide.

74. The kit according to claim 73, wherein the multi-antigenic domain encoded in the genome of the vesicular stomatitis virus (VSV) comprises the amino acid sequences of each of the antigens, or fragments or sequence variant thereof, which are contained in the multi-antigenic domain of the peptide.

75. The kit according to claim 72, wherein the multi-antigenic domain of the peptide comprises an antigen, or a fragment or sequence variant thereof, which is contained in the multi-antigenic domain encoded in the genome of the vesicular stomatitis virus (VSV).

76. The kit according to claim 75, wherein the multi-antigenic domain of the peptide comprises the amino acid sequences of each of the antigens, or fragments or sequence variant thereof, of the multi-antigenic domain which is encoded in the genome of the vesicular stomatitis virus (VSV).

77. The kit according to claim 72, wherein
the multi-antigenic domain of the peptide comprises an amino acid sequence according to SEQ ID NO: 13 or 14, or a sequence variant thereof having at least 70% sequence identity, and
the multi-antigenic domain encoded in the genome of the vesicular stomatitis virus (VSV) comprises the amino acid sequence according to SEQ ID NO: 19 or SEQ ID NO: 48, preferably the amino acid sequence according to SEQ ID NO: 19, or a sequence variant thereof having at least 70% sequence identity.

78. The kit according to claim 72, wherein
the peptide comprises an amino acid sequence according to SEQ ID NO: 17, SEQ ID NO: 18, SEQ ID NO: 46, or SEQ ID NO: 47, preferably an amino acid sequence according to SEQ ID NO: 17 or 18, or a sequence variant thereof having at least 70% sequence identity, and
the vesicular stomatitis virus (VSV) comprises an RNA genome comprising an RNA sequence according to SEQ ID NO: 30, or a sequence variant thereof having at least 70% sequence identity.

79. The kit according to claim 72, further comprising an inhibitor of the PD-1/PD-L1 pathway.

80. The kit according to claim 79, wherein the inhibitor of the PD-1/PD-L1 pathway is selected from the group consisting of pembrolizumab; nivolumab; pidilizumab; cemiplimab; PDR-001; atezolizumab; avelumab; durvalumab, ezabenlimab, an antibody comprising a heavy chain comprising the amino acid sequence of SEQ ID NO: 31 and a light chain comprising the amino acid sequence of SEQ ID NO: 32; an antibody comprising a heavy chain comprising the amino acid sequence of SEQ ID NO: 33 and a light chain comprising the amino acid sequence of SEQ ID NO: 34; and an antibody comprising a heavy chain comprising the amino acid sequence of SEQ ID NO: 35 and a light chain comprising the amino acid sequence of SEQ ID NO: 36.

81. A combination comprising:
(i) a peptide comprising
a) a cell penetrating peptide;
b) a multi-antigenic domain comprising at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); and
c) a TLR peptide agonist; and
(ii) a vesicular stomatitis virus (VSV) encoding a multi-antigenic domain comprising at least one tumor antigen, or fragment or sequence variant thereof, wherein the tumor antigen is encoded in a 5′-upstream open reading frame (uORF) within the 5′ UTR of KRAS mRNA (KRAS-uORF), of TPX2 mRNA (TPX2-uORF) or of AURKA mRNA (AURKA-uORF).

82. The combination according to claim 81, wherein the multi-antigenic domain encoded in the genome of the vesicular stomatitis virus (VSV) comprises an antigen, or a fragment or sequence variant thereof, which is contained in the multi-antigenic domain of the peptide.

83. The combination according to claim 82, wherein the multi-antigenic domain encoded in the genome of the vesicular stomatitis virus (VSV) comprises the amino acid sequences of each of the antigens, or fragments or sequence variant thereof, which are contained in the multi-antigenic domain of the peptide.

84. The combination according to claim 81, wherein the multi-antigenic domain of the peptide comprises an antigen, or a fragment or sequence variant thereof, which is contained in the multi-antigenic domain encoded in the genome of the vesicular stomatitis virus (VSV).

85. The combination according to claim 84, wherein the multi-antigenic domain of the peptide comprises the amino acid sequences of each of the antigens, or fragments or sequence variant thereof, of the multi-antigenic domain which is encoded in the genome of the vesicular stomatitis virus (VSV).

86. The combination according to claim 81, wherein
the multi-antigenic domain of the peptide comprises an amino acid sequence according to SEQ ID NO: 13 or 14, or a sequence variant thereof having at least 70% sequence identity, and
the multi-antigenic domain encoded in the genome of the vesicular stomatitis virus (VSV) comprises the amino acid sequence according to SEQ ID NO: 19 or SEQ ID NO: 48, preferably the amino acid sequence according to SEQ ID NO: 19, or a sequence variant thereof having at least 70% sequence identity.

87. The combination according to claim 81, wherein
the peptide comprises an amino acid sequence according to SEQ ID NO: 17, SEQ ID NO: 18, SEQ ID NO: 46, or SEQ ID NO: 47, preferably an amino acid sequence according to SEQ ID NO: 17 or 18, or a sequence variant thereof having at least 70% sequence identity, and
the vesicular stomatitis virus (VSV) comprises an RNA genome comprising an RNA sequence according to SEQ ID NO: 30, or a sequence variant thereof having at least 70% sequence identity.

88. The combination according to claim 81, further comprising an inhibitor of the PD-1/PD-L1 pathway.

89. The combination according to claim 88, wherein the inhibitor of the PD-1/PD-L1 pathway is selected from the group consisting of pembrolizumab; nivolumab; pidilizumab; cemiplimab; PDR-001; atezolizumab; avelumab; durvalumab, ezabenlimab, an antibody comprising a heavy chain comprising the amino acid sequence of SEQ ID NO: 31 and a light chain comprising the amino acid sequence of SEQ ID NO: 32; an antibody comprising a heavy chain comprising the amino acid sequence of SEQ ID NO: 33 and a light chain comprising the amino acid sequence of SEQ ID NO: 34; and an antibody comprising a heavy chain comprising the amino acid sequence of SEQ ID NO: 35 and a light chain comprising the amino acid sequence of SEQ ID NO: 36.

90. A method for ameliorating, treating, or reducing the risk of occurrence of a cancer, or for inducing or enhancing an anti-tumor response in a subject in need thereof, the method comprising administering to the subject an effective amount of:
(i) a composition comprising:
a) at least one tumor antigen, or a fragment or sequence variant thereof,
wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2);
b) a nucleic acid encoding said tumor antigen;
c) an antigen-presenting cell (APC) containing (a) or (b); or
d) a T-cell expressing either a T-cell receptor or a CAR T-cell receptor targeting said tumor antigen;
(ii) a peptide comprising:
a) a cell penetrating peptide;
b) a multi-antigenic domain comprising at least one tumor antigen, or a fragment or sequence variant thereof, wherein the tumor antigen is encoded in a 5′-upstream open reading frame (uORF) within the 5′ UTR of the KRAS mRNA (KRAS-uORF1), of the TPX2 mRNA (TPX2-uORF1) or of the AURKA mRNA (AURKA-uORF2); and
c) a TLR peptide agonist;
(iii) a vesicular stomatitis virus (VSV) encoding a multi-antigenic domain comprising at least one tumor antigen, or fragment or sequence variant thereof, wherein the tumor antigen is encoded in a 5′-upstream open reading frame (uORF) within the 5′ UTR of KRAS mRNA (KRAS-uORF), of TPX2 mRNA (TPX2-uORF) or of AURKA mRNA (AURKA-uORF);
(iv) a vaccine comprising (i), (ii), and/or (iii);
(v) a kit comprising (ii) and (iii); and/or
(vi) a combination comprising (ii) and (iii).

91. The method according to claim 90, wherein the cancer is a cancer of the gastrointestinal tract (GI).

92. The method according to claim 90, wherein the cancer is selected from the group consisting of anal cancer; appendix cancer; cholangiocarcinoma/bile duct cancer; gastrointestinal carcinoid tumor; colorectal cancer, colon cancer, rectal cancer; esophageal cancer; gallbladder cancer; gastric (stomach) cancer; gastrointestinal stromal tumor (GIST), and pancreatic cancer.

93. The method according to claim 92, wherein the cancer is selected from the group consisting of colon cancer, rectal cancer, colorectal cancer, and pancreatic ductal adenocarcinoma.

94. The method according to claim 90, comprising administering the peptide and the vesicular stomatitis virus (VSV) to the subject.

95. The method according to claim 94, wherein the peptide and the vesicular stomatitis virus (VSV) are each administered at least once.

96. The method according to claim 95, wherein the peptide is administered prior to the administration of the vesicular stomatitis virus (VSV).

97. The method according to claim 94, wherein the peptide is administered at least twice.

98. The method according to claim 97, wherein the peptide is administered prior to and subsequent to the administration of the vesicular stomatitis virus (VSV).

99. The method according to claim 94, wherein the peptide and the vesicular stomatitis virus (VSV) are administered in the order K-V-K, K-V-K-K, K-V-K-K-K, or K-V-K-K-K-K.

100. The method according to claim 90, comprising a single administration of the vesicular stomatitis virus (VSV).

101. The method according to claim 94, wherein the peptide and the vesicular stomatitis virus (VSV) are administered via a same route.

102. The method according to claim 94, wherein the peptide and the vesicular stomatitis virus (VSV) are administered via a different route.

103. The method according to claim 94, wherein the peptide and the vesicular stomatitis virus (VSV) are administered intravenously, subcutaneously, or intramuscularly.

104. The method according to claim 94, wherein the peptide is administered subcutaneously, and the vesicular stomatitis virus (VSV) is administered intravenously or intratumorally.

105. The method according to claim 90, further comprising administration of an inhibitor of the PD-1/PD-L1 pathway.

106. The method according to claim 105, wherein the inhibitor of the PD-1/PD-L1 pathway is administered concomitantly, sequentially or alternately with the peptide or the vesicular stomatitis virus (VSV).


SEQUENCE LISTING (51 sequences)

SEQ ID NO: 1 - protein, 23 aa, Homo sapiens
VAAARPVLPAPAISDWERARRRH

SEQ ID NO: 2 - protein, 28 aa, Homo sapiens
VAAAKVAAARPVLPAPAISDWERARRRH

SEQ ID NO: 3 - protein, 38 aa, Homo sapiens
VGRKEGAARARVSLLPEFGTAEVHLPAPSAVRAARPGF

SEQ ID NO: 4 - protein, 19 aa, Homo sapiens
TAEVHLPAPSAVRAARPGF

SEQ ID NO: 5 - protein, 22 aa, Homo sapiens
GGDKGRLVGVAERQVPCRFLRP

SEQ ID NO: 6 - protein, 38 aa, synthetic construct
  REGION 1..38: note = CEACAM5 fragment
NRTLTLFNVTRNDARAYVSGIQNSVSANRSDPVTLDVL

SEQ ID NO: 7 - protein, 19 aa, synthetic construct
  REGION 1..19: note = CEACAM5 fragment
PDSSYLSGANLNLSCHSAS

SEQ ID NO: 8 - protein, 63 aa, synthetic construct
  REGION 1..63: note = CEACAM5 fragment
PQYSWRINGIPQQHTQVLFIAKITPNNNGTYACFVSNLATGRNNSIVKSITVSASGTSPGLSA

SEQ ID NO: 9 - protein, 120 aa, synthetic construct
  REGION 1..120: note = CEACAM5 fragment fusion construct
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSA

SEQ ID NO: 10 - protein, 33 aa, synthetic construct
  REGION 1..33: note = DUOXA2 fragment
TGVLSLFLGGAVVSLQYVRPSALRTLLDQSAKD

SEQ ID NO: 11 - protein, 24 aa, synthetic construct
  REGION 1..24: note = KRAS-G12D fragment
TEYKLVVVGADGVGKSALTIQLIQ

SEQ ID NO: 12 - protein, 24 aa, synthetic construct
  REGION 1..24: note = KRAS-G12V fragment
TEYKLVVVGAVGVGKSALTIQLIQ

SEQ ID NO: 13 - protein, 260 aa, synthetic construct
  REGION 1..260: note = multi-antigenic domain ATP150
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKD

SEQ ID NO: 14 - protein, 260 aa, synthetic construct
  REGION 1..260: note = multi-antigenic domain ATP152
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGAVGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKD

SEQ ID NO: 15 - protein, 42 aa, synthetic construct
  REGION 1..42: note = cell penetrating peptide (Z13)
KRYKNRVASRKSRAKFKQLLQHYREVAAAKSSENDRLRLLLK

SEQ ID NO: 16 - protein, 35 aa, synthetic construct
  REGION 1..35: note = TLR agonist (Anaxa)
STVHEILSKLSLEGDHSTPPSAYGSVKPYTNFDAE

SEQ ID NO: 17 - protein, 338 aa, synthetic construct
  REGION 1..338: note = ATP150
MKRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKDSTVHEILSKLSLEGDHSTPPSAYGSVKPYTNFDAE

SEQ ID NO: 18 - protein, 338 aa, synthetic construct
  REGION 1..338: note = ATP152
MKRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGAVGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKDSTVHEILSKLSLEGDHSTPPSAYGSVKPYTNFDAE

SEQ ID NO: 19 - protein, 285 aa, synthetic construct
  REGION 1..285: note = multi-antigenic domain VSV-GP154
MNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQTEYKLVVVGAVGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKD

SEQ ID NO: 20 - protein, 18 aa, synthetic construct
  REGION 1..18: note = multi-antigenic domain Mad25
QAEPDRAHYNIVTFSSKS

SEQ ID NO: 21 - protein, 30 aa, synthetic construct
  REGION 1..30: note = cell penetrating peptide (Z14)
KRYKNRVASRKSRAKFKQLLQHYREVAAAK

SEQ ID NO: 22 - protein, 17 aa, synthetic construct
  REGION 1..17: note = cell penetrating peptide (Z15)
KRYKNRVASRKSRAKFK

SEQ ID NO: 23 - protein, 19 aa, synthetic construct
  REGION 1..19: note = cell penetrating peptide (Z18)
REVAAAKSSENDRLRLLLK

SEQ ID NO: 24 - protein, 35 aa, synthetic construct
  REGION 1..35: note = TLR agonist (Anaxa variant)
STVHEILCKLSLEGDHSTPPSAYGSVKPYTNFDAE

SEQ ID NO: 25 - protein, 498 aa, synthetic construct
  REGION 1..498: note = GP of LCMV
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 26 - protein, 422 aa, vesicular stomatitis virus
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 27 - protein, 265 aa, vesicular stomatitis virus
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 28 - protein, 2109 aa, vesicular stomatitis virus
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 29 - protein, 229 aa, vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 30 - RNA, 12057 nt, synthetic construct
  misc_feature 1..12057: note = VSV-GP154 (complement without reverse)
  source 1..12057: mol_type = other RNA
tgcttctgtttgtttggtaataatagtaattttccgagtcctctttgaaattgtcattagttttacagacaatgtcagttctcttagtaactgttgtgtcagcatcaaggttttgaaggacgtttactcctaggtcaccttatgggccgtctaatgaagtcttttagtttcctctaaggagaaatgtagttatgatgtttttcaaacagtctagattctcctatacagatggttccggagtttaggcctttacatagttagtatgtacagttgtcgatgaacatacctcgtaatttcctgtaggccccattcaacctatttctaaccagttcaaagccttatttgtagccctttcgtcccctatgttagccttataaactggaacataggaactttcgggacctgccgcatgaaggtctacctcatagcctacgaaggtcttggtcgcgtctactgtttaccaacggaaacatagatgaaccgaatatgtctcacccgtcttgtgtttacggacttatgtcttttttcgagtacctacccgactgtttagttacgttttactagttacttgtcaaacttggagaacacggtcttccagcactgtaaaaactacacacccctttactgtcattaatgtgtttttaacagcgacgtcacctgtacaagaaggtgtacaagttttttgtacttacacggagcaagtctatgccttgataacaaaggtctaagtttctaacacgacgtaaccgttgtaaacctgtggagacgttttattggccttacagatgtcttctacattgctggacctagaacttggctcttcaacgtctactttaccaggtttactacgaaggtccggttctttaactgttccggctaagtatgtacggaataaactagctgaaacctaacagaagattcagaggtataagaaggcagtttttgggacggaaggtgaagacccccgttaactgtcgagaagacgagtctaggtggtctcgttccttacgggctgtcggactactgtaactcatatgtagagaatgatgtcgtccaaacaacatgcgaatacgtcatcctaggagacggctgaaccgtgttgtcaaaacacaacctctattgtttatgtgaggtctactatcatggcctcctaactgctgattacgtggcggtgttccgtctctacaccagcttaccgagcctaccaaacttctagttttgtcttttggctgaggactatactacgtcatacgcttttctcgtcagtacagtgacgttccggattctctcttctgttaaccgttcatacgattcagtcttaaactgtttactgggatattaagagtctagtggataatatataatacgatgtatactttttttgattgtctatagtacctattagagtgttttcaagcactcatagagttcaggataagagcagacctagtccgccatcctctctatctactctagcttcgtgttgctcgacttttcaggttaatactcaacaaggttctcctacctcaccttctcgtatgattcgggagaataaaagtccgtcgtctactaagactgtgtcttagacttggtctttaacttctgttagttccgaacatacgtggtctaggtcttcgactcgttcaacttccgaaatatgtccccggaaatctactgatacgtctactccttcacctacaacataaatgaagcctgacctttgtcggactcgaacttagactgctcgtacctttctggaatgccaactgtagcggtctcccaaattcacctctcgtctttagggtcaccgaaagctgctaatttcgtcagcacgtttcacggtttatgaccttagaccgtctcacgtgtaaacttcgtagccctcttccccagtaatacttcctcgcggtctattgaggcctacatatattccagtgaggtcactacttgtgtgtaggcagggttagtcttcgtcatagtctacaaaccagagagagtttctgtaggtactgaaaggttgggttctttcgttcagaagtcggagagtggtataggaacctacttaacaagagtagatctcctctcaagtagagacagcctccactgcctgcttacagagtatttctccggtaggacgagccggactctatgtttttcaacatgttagtccgctctcagtttataagagacatctgatactttttttcattgtctatagtgctagattcacaatagggttaggtaagtagtactcaaggaatttcttctaagagccagacttcccctttccattctttagattctttaatccctagcgtggtgggggaatacttctcctgtgatcgtacctcatacgaggctcgcgaggttaactgtttaggataaaacctcaactgctctacctgtggatactaggcttagttaattctatactctttaagaagaaatgtcacttttactgccaatctagattagcaggcaagtcttgtatgagtctacaccgtcggcgacatagggtaaccctagtgtacatgtagccttaccgtccctttgcagggaagatgttttagaaccgaaaaaacccaagaagattagatttccggtgaggtcgccataaccgtctagttccagttggtctcatagtgcgagtgacgcttccgtcccgaataaacggtgtatcctaccccttctggggagggtacgagttacatggtctcgtgaagtcttctggtaagttatatccagaaatgttcccttgctaactcgagtgttactggtagatgctactactcagtgaccttcgtcgaggatactagaccctagtaaagttaagaaggtttaaaagactaaagtctctcttccggaattacaaaccggactaacagctctttttccgtagacctcgcacccaggacctgagatagccggtgaagtttactcgatcagattgaagatcgaagacttgttaggggccaaatgagtcagaggggattaaggtcggagagcttgttgattataggacagaaaagatagggatactttttttgattgtctctagctagacaaatgcgcagtgcctagggggcccgacgtccttaagcggtggtacccggtctagcactggtacaagctccgggacggggtgtagtagctgctccactagttgtagcactagtagcacgagtagtagtagtggtcgtagttccggcacatgttgaagcggtggacgccgtaggaccgggaccactcgaaggacaaggaccggccgtcttcgacgccgtacatgccggacttaccggggctatagatgttcccgcacatggtcaagttctcgcacctcaagctgtactcggtggacttggagtggtacgggttgcggacgtcgcggttgttatcggtggtgatgtagtcgtacccgtcgtcgccggacctcaactggaagtggttgctgtcgtaggacttggtgttgaagacgttggagtggtcgcggaagttgttcttttggaagctggtgtgggagtactcgtagcactcgtcggacgtggactcgtagtctccgttgtcgttggtgttccggcactcgacgctgaagttgttgccgtagtggtaggtcatgttggactcgaagtcgctaggagtctcgcggtagtcggtcacgtcttggaagtctccgtctcacgacctgtacaagtcttggcggaagccgccgttcatgtactcttcgccgaccccgacccggccgtcgctgccgttctggtggaccacgtcggtctggtcgatggtcatggagtagtaggtcttgtcttggaccctcttggtgacgtctatgcggcctggaaagccgtactcgtcttaggacaagcgggtcctcttttggttcaaggagtggtcctctgaccggccgtggaagtggacctgggactcgctgtcgtcgccgcacctcttgggaccgccgatgacggagtggttcacctactaggaccggcggctcgacttcacgaagccgttgtggcggcaccggttcacgttgcacttggtgctgctcctcaagacgctgtacgactctgagtagctgatgttgttccggcgggactcgttcaagttcgtcctgcacctctcgcgggacgtgcacaagttctggtggcacttgtcggagtagtcgctggtcgacgagtactctttggtggactctctggagtacccgcacgggatgacgttgatgtcgttcaagaccatagacctcgtgcggttctggccgctctggtcgcacgggttcacgaccgaccactggttaccgtcgatggacttgctctgggtgaagtcgctggtctagctcgtccttcggctgttgtactagtggctctacgactccttcctgatgtagttctctgtcccgtcgtggggggaccgggagtacctagacgagtacaagtcgtggtcgcggatggagtagtcgtagaaggacgtggaccacttctaggggtgggtgtctgtgtagttcccgccgtcgacggggttcggggtgtctgagtggttgttcccgtagacgtcgacgccgcggaagttccacgggccgcacttttggtagaccttctcctctattcgccggcgatgctggagctgatactttttttgattgtctataggagctgcggtggtacttggcctgtgactgggacaagttgcactgggccttactgcggtctcggatgcacaggccgtaggtcttatcgcacaggcggttatcttcgctggggcactgtgacctgcacgacggactatcgtcgatagactcgccgcggttggacttggactcgacggtaagacggagaggtgtcatgtcgaccgcctagttgccttagggagtcgtcgtgtgggtccacgacaaatagcggttctagtggggattgttgttgccgtggatgcggacgaagcacaggttagaccggtggccgtctttgttgtcgtagcacttcaggtagtggcacagacggtcgccgtgtagaggacctgactcgcggtgtctcatgttcgaccaccaacaccctcgactgccgcaccctttttcgcgggactgttaggtcgactaggtctggctcatatttgagcaccagcagccgcgacaaccgcaaccgtttagacgagactggtaagtcgagtaagtccaccggcgacggtctggacaagaaggacgaggacggtagtcgctgaccctttctcgatcttcttccgtgcagccggcctttcttccgcggcgatctcgatctcaatcggacgacggtctcaaaccgtggcggcttcaagtagacggacgaggatcgcgacagtctcggcggtctggtcctaaaccgcctctattcccgtctgagcacccgcaacgtctttctgtccacgggacgtctaaggactccggttgtccgcacgactcggacaaagaaccgcctcgacaccacagggacgtcatgcagtctggatcgcgagactcttgggacgacctagtctcgcggttcctaactagttgatcggtctaagaagtacaaacctggtttagttgaacactatggtacgagtttctccggagttaatataaactcaaaaattaaaaatactttttttgattgtcgttagtaccttcaggtgctaaaactctggctgctcaagttactaaagttacttctactgatacggtgttctcttaaggacttagggctactcgcgtactgcatgaacttagtacgactaatgttggacttaagaggagattaatcactactataactgttaaattagtcctttaagttaagagaaggttaagggagctacaccctatcattcttgaccctacctcaagaactctacaattgcagtacagttcggttagggtagggttgtagagtctacgtatttacctacccttcaaccaattacagactattagtactacggtcagttcccatatcaaaaaatgtacttcacctgtttctccgtctttattgtaaactgcaccacctctggaagtaggcgccgaccccgttgtttggttaacttatgtagtttttcctttctacctgactgagtaagttttaagagcgaataaacacagttttcaaaaacctgaatgtgttcaactgtaattagaatttacgacagagactccaccttaacgagttgaaccgctcctgaaagtttccgtttcagtcttcttcaagagtaccttgcttgtatacgtcctaatcccaagggtcgaacccaggatgaaaataaagtcttcctacccgaatgaagttctttgaactataagattacctggctttgaaagacaattaccagtttctacactaatatccctcctacgtttgccacgataggtaccatacatcttatctgttggacaagagtctcgttctgtagaagagggaagatttatagatgtcttaacctctattttaacacctctccgtccctttaaaaagaatactgaactaattttaccaccttggctatacgttgaacttcgactactttaatcgttctcttagttccggaaatcagggtgttaagggagtaaaacttttagtatagttctgaagacaactacttccccgtttttaactggctccatattctaaggaggtactagtctattactcacacttttgtcacctagagtgtgaccactaaatacctagcaagtctgtaaccccagtaggaaaatatctaataatgtgacctgatctttttaatgtaagggttcattggtacttctttctataactacacagtatacgttttcgtgaacgttcactaaatcgagcctaacaagataaagttgtcaagttactagtatttttcaccaagcacttacctctgaacgagggagtactagtagggaaattttcagtacaatttcttttatgtaccgggtgtcgacgagttcaagttctaaaacctctatttaccgtacttgaaggcgactaatttacaaaactttatgggctgaatgatctgggtagctattatatgagactgttttcagtaagttacttatccagtctccacaactttgtacaggcttacttaggcttgtgaggatagggatcatttttccacaacgtctgatacaacctgtgtttccgatggttaacctttcttaaagaatttctctaactactcttcccgaatctactactactagattaataaccagaatttcctttcctctcccttgacttcaaccgtccatctaaaaagagggattacagaacctttaacgctcttatgaaacattaatggcttataaactatttctgagtaaagcagggatacaaatttccggactgttaccgcctgctagattgacgtcagtaatttttctacaatctaaggagtaggccggttcctaacttcagtatactccgttaaacgtatcggttagtgtaactaatgctttttaccttattggtggtttccttcaatagtttgccgggtcacaaggctcaatacccggtcaagaatccaataggtaggaattagctctcttgagtacttaaaaaactcttttcagaatatatgatgttaccttctggtctgaactacgcacaagtgttgttgtgtgactagttaagttggagggttgctcaaacaaccgttcctgttctcccacctgaccttccagatgccgtttttcctacctcataggagttagatgaccaataagtttctctccgattttagtctttgtgacgacagtttcagaaccgtgttccactattagttcaataaacgtgtgtcatattttgcttctttagctctttgcaacatcttaatgtcccacgagagttagtttaccaaagattattactcttttaatactgacgttagttttatccctgtcccttcaatcctgaaaactatttactgctactctgatacgttagacgtctaatgaacttaatacctttttatggctaaaaggcacctcactaatctcccaatctctggttctctaccagtgctcactgaacacagtggttactggtttatgggtgaacacgattatattactcgagtcaaaggtgtttacgagagtggcatcgagtaaaacgactcttgggttagttacggtactatgtcatgttaataaaaccctgtaaacgatctgagaacaactactacgtactaggacgagaagcagttagtaacatacttcaagttctattctatggcccgaacgtgtcaagatgaaagtttatgcggtacaacataaacctgggaaggtaacctcctcacagcccgtacagaaacaggtccaaaaactaatctcggaagggtctagggcattgtctttcagagagtaagacctctaagtaggtacatgtacgagcttcactcgtagacttcctctactcacgtcataaacctttggggctctatcggttcaaagcttattgagtgtatctgttcgatcatcttctaggttggagagacttgtagcgatacccttactcaggtcgcttgaacaatttctgactccaattttttacgaattagcttagttctgtttggtagtccttggtccactaattcctacgttggtatataaacatagtacttctcctagccgagtcttcaaagaataccagttatttaggagacaagggatctaaaaattcacttaagtttagtccgtgaaaaaaccctcagcgtctgcccgagtagtcagataaagttttaagagcatgataagccttgaggaaattctttttcatagtatcccttaacctactaaactaacactcctcactccataggagaaactgtgtaaatccctttgaagtaaactcttcccctagtacattttacacctgtacaagtcgatgagtacgactgtgtaattctatgtttaggaccccggcatgtcaataaccctgttgacatggggtaggtaatctttacaacccaggtgttgtagcttttctctgaggaacacgtggtacattgtgtagtcccaagttaatacaaagacacgtaacaggtctgccctaggtactgcagaaatcaagtgcccctggtaacggacgaatagatcccagattttgtagacttagatgtagataaaacgtcggaaccctttccctttcgtttcagggtgactaattttctcgatgtgcagaatctctacgatagagaaccaaacaacttgggctgagatttgatcgttactgatatgaaagattgtaggtgagaaattgtccgcttcttacctggttttccgtcgtacccaagttttcttgtcccagacgggaagtatccaaaagctgtagagcctactcggtaccacccaagcgtagagtctcgtgacgtcgtaactggtccaactaccgttgatgtctgtggtactccctagaccctctagtcttaaagctgaaaaataaggttcgttgcaacgagatacgagtttaatggtggtgacaacgttctctgcctacctagtggtcaacatgtctagtaatagtataacggacattcaggacaaactctgggtatcttctctagtgggacctgagttcatacctgatgtgcgggggtctacatagggtacacgacttctgtacctccttaccccttccaagcacccctgttctctattttgtctagataggaaatcttcccttaaccttcttaaatcgtggacgactcgttaggatagttcagccgtctacatatccaaaagatatacctctgaaccgcatatcttttagatgagtacggctcctgtcaagagataaaggagatagatatgttccagcataatctccagctccaaagaattttcccaacgatctgcctaattactctcgttcaacgacggttcattatgtggcctcttcagaccgagtaaacttctccggccggttgcgtcacatgcctccaaactaaatgaactaactatttaactcacatagtggaggtaaggaaagagaatgatctagtcctggataatctctgcttaatctttgctaaggggtgttctagggttggaggataggctgttcgttggcactatacccccactaacagtctttaatgaagtttatggttacggcagattaacttttcccttttatgtctagtgtaataagtgttaataccaataagagtctacagaataggtatctgaagtaacctggtaagagataaaggtggtgggagaacgtttaggatatgttcggtaaaaatagaccctttctattcttactcaactctctcgaccgtttagaaagaagtaacgattctagtcctctccccacccttctgtatgtacactttaagaagtggttcctgtataataacacaggtctcctttagtctgtacgaacgttcaagccctaacgattcctattattatttctgtactcgatagggggaaccccttcccttaggtctccctgttaatgttgttagggacaaataatatgctggtggggaatgggtttctacgatctctacggaggttcttaggttttaggggacgacaggccttagtccaacccggttaatggttgaccgcgagtaatattttaagcctcatataatgtaccttacccttaggtaatgtccctgaagaactcaacacctctgccgaggcctccctactgacgacgtaatgatgctcttttacacgtatcgtctccttataagttatcagacaatcttaatagtcccagtcagtacgctccgcggagaggactcggggggtcacgggatctttgaaatcctcctctatttagctctacacatttaccactttgtacaacccttataggtagactgaatacactgggttcctgaaccctgataaaggaggctgagtttcgtccgaaccccgaagtttaactaaattaacattacctataccttcaagccctaagaagatgatcggacttttaactctgcttacaatctttaatacacgtggcctaaaacctactcgttcctcaaaattagatgttctgaataccttgtatataaacactctcgcttttcttacgtcattgttaggaaccagggtacaagttctgccagctgaatcaagtttgtcttaaatcatcaagagtttgcagacttcatatataccatacatttccaaacttctttaattagctacttgggttagggctaaccagaaggtagttacttaggacctttttggacatgcgtaaggtcagtagtcttgtccttaaacggtctcgtttcttccaatcatgtatgaaatggaactgtccataagggagggttaagtaaggactaggaaaacatttgtaactctgatacgatgtttataagcctcatgggtgcccacacagagtacgccgacggaattttagtagactatctggacgtctaaataactggtaatcggaaaaaatataccgctaatatagcataatattgtagttagtatagtctcatcctggctatggaggcttggggggtagtctaccttaacgtgttttacacccctagcgatattgaccatattcgaaaaccgactcaaactacctctttctgtaaggtgatatagttgtcacaaatcgtcaataggtcgttagtaagggctaatccaccctccgacaaagtcattttcctcctatgttcgtcttcacctcatgatctccactacccgagggttttctatgggcttaaagtctgaggaaccggggttagcccttgacctagtctagagaccttaaccaggctttggttcaagcagatttaggtaagttactctagaacaagttagtcgatacagcatgtcacctattagtaaactttaccagtttaaacgcttctttgtgtccttactaacttacctagttatctgcttaaagttttcttctggccagatatgactacaacttctcactggatgtgctccttttgagaacctctctaattttttagtactcctctgaggtttgaaattcatactttttttgaaactaggaattctgggagaacaccaaaaataaaaaatagaccaaaacaccagaagca

SEQ ID NO: 31 - protein, 446 aa, synthetic construct
  REGION 1..446: note = heavy chain of anti-PD1 antibody PD1-1
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 218 aa, synthetic construct
  REGION 1..218: note = light chain of anti-PD1 antibody PD1-1
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 446 aa, synthetic construct
  REGION 1..446: note = heavy chain of anti-PD1 antibody PD1-2
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 218 aa, synthetic construct; identical to SEQ ID NO: 32
  REGION 1..218: note = light chain of anti-PD1 antibody PD1-2

SEQ ID NO: 35 - protein, 446 aa, synthetic construct
  REGION 1..446: note = heavy chain of anti-PD1 antibody PD1-3
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 36 - protein, 218 aa, synthetic construct
  REGION 1..218: note = light chain of anti-PD1 antibody PD1-3
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 69 bp, Homo sapiens
  source 1..69: mol_type = other DNA
gtggccgctgccagacctgttcttcctgctcctgccatcagcgactgggaaagagctagaagaaggcac

SEQ ID NO: 38 - DNA, 114 bp, Homo sapiens
  source 1..114: mol_type = other DNA
gtcggccggaaagaaggcgccgctagagctagagttagcctgctgccagagtttggcaccgccgaagttcatctgcctgctcctagcgctgtcagagccgccagaccaggattt

SEQ ID NO: 39 - DNA, 66 bp, Homo sapiens
  source 1..66: mol_type = other DNA
ggcggagataagggcagactcgtgggcgttgcagaaagacaggtgccctgcagattcctgaggcca

SEQ ID NO: 40 - DNA, 114 bp, synthetic construct
  misc_feature 1..114: note = CEACAM5 fragment
  source 1..114: mol_type = other DNA
aaccggacactgaccctgttcaacgtgacccggaatgacgccagagcctacgtgtccggcatccagaatagcgtgtccgccaatagaagcgaccccgtgacactggacgtgctg

SEQ ID NO: 41 - DNA, 57 bp, synthetic construct
  misc_feature 1..57: note = CEACAM5 fragment
  source 1..57: mol_type = other DNA
cctgatagcagctatctgagcggcgccaacctgaacctgagctgccattctgcctct

SEQ ID NO: 42 - DNA, 189 bp, synthetic construct
  misc_feature 1..189: note = CEACAM5 fragment
  source 1..189: mol_type = other DNA
ccacagtacagctggcggatcaacggaatccctcagcagcacacccaggtgctgtttatcgccaagatcacccctaacaacaacggcacctacgcctgcttcgtgtccaatctggccaccggcagaaacaacagcatcgtgaagtccatcaccgtgtctgccagcggcacatctcctggactgagcgcc

SEQ ID NO: 43 - DNA, 66 bp, synthetic construct; identical to SEQ ID NO: 39
  misc_feature 1..66: note = DUOXA2 fragment
  source 1..66: mol_type = other DNA

SEQ ID NO: 44 - DNA, 72 bp, synthetic construct
  misc_feature 1..72: note = KRAS-G12D fragment
  source 1..72: mol_type = other DNA
acagagtacaagctggtggttgtgggagctgacggcgtgggaaaaagcgccctgacaatccagctgatccag

SEQ ID NO: 45 - DNA, 72 bp, synthetic construct
  misc_feature 1..72: note = KRAS-G12V fragment
  source 1..72: mol_type = other DNA
accgagtataaactcgtggtcgtcggcgctgttggcgttggcaaatctgctctgaccattcagctcattcag

SEQ ID NO: 46 - protein, 337 aa, synthetic construct
  REGION 1..337: note = ATP150 (v2)
KRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKDSTVHEILSKLSLEGDHSTPPSAYGSVKPYTNFDAE

SEQ ID NO: 47 - protein, 337 aa, synthetic construct
  REGION 1..337: note = ATP152 (v2)
KRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGAVGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKDSTVHEILSKLSLEGDHSTPPSAYGSVKPYTNFDAE

SEQ ID NO: 48 - protein, 284 aa, synthetic construct
  REGION 1..284: note = multi-antigenic domain VSV-GP154 (v2)
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQTEYKLVVVGAVGVGKSALTIQLIQVAAARPVLPAPAISDWERARRRHVGRKEGAARARVSLLPEFGTAEVHLPAPSAVRAARPGFGGDKGRLVGVAERQVPCRFLRPTGVLSLFLGGAVVSLQYVRPSALRTLLDQSAKD

SEQ ID NO: 49 - DNA, 12057 bp, synthetic construct
  misc_feature 1..12057: note = cDNA sequence of VSV-GP154
  source 1..12057: mol_type = other DNA
acgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcacggatcccccgggctgcaggaattcgccaccatgggccagatcgtgaccatgttcgaggccctgccccacatcatcgacgaggtgatcaacatcgtgatcatcgtgctcatcatcatcaccagcatcaaggccgtgtacaacttcgccacctgcggcatcctggccctggtgagcttcctgttcctggccggcagaagctgcggcatgtacggcctgaatggccccgatatctacaagggcgtgtaccagttcaagagcgtggagttcgacatgagccacctgaacctcaccatgcccaacgcctgcagcgccaacaatagccaccactacatcagcatgggcagcagcggcctggagttgaccttcaccaacgacagcatcctgaaccacaacttctgcaacctcaccagcgccttcaacaagaaaaccttcgaccacaccctcatgagcatcgtgagcagcctgcacctgagcatcagaggcaacagcaaccacaaggccgtgagctgcgacttcaacaacggcatcaccatccagtacaacctgagcttcagcgatcctcagagcgccatcagccagtgcagaaccttcagaggcagagtgctggacatgttcagaaccgccttcggcggcaagtacatgagaagcggctggggctgggccggcagcgacggcaagaccacctggtgcagccagaccagctaccagtacctcatcatccagaacagaacctgggagaaccactgcagatacgccggacctttcggcatgagcagaatcctgttcgcccaggagaaaaccaagttcctcaccaggagactggccggcaccttcacctggaccctgagcgacagcagcggcgtggagaaccctggcggctactgcctcaccaagtggatgatcctggccgccgagctgaagtgcttcggcaacaccgccgtggccaagtgcaacgtgaaccacgacgaggagttctgcgacatgctgagactcatcgactacaacaaggccgccctgagcaagttcaagcaggacgtggagagcgccctgcacgtgttcaagaccaccgtgaacagcctcatcagcgaccagctgctcatgagaaaccacctgagagacctcatgggcgtgccctactgcaactacagcaagttctggtatctggagcacgccaagaccggcgagaccagcgtgcccaagtgctggctggtgaccaatggcagctacctgaacgagacccacttcagcgaccagatcgagcaggaagccgacaacatgatcaccgagatgctgaggaaggactacatcaagagacagggcagcacccccctggccctcatggatctgctcatgttcagcaccagcgcctacctcatcagcatcttcctgcacctggtgaagatccccacccacagacacatcaagggcggcagctgccccaagccccacagactcaccaacaagggcatctgcagctgcggcgccttcaaggtgcccggcgtgaaaaccatctggaagaggagataagcggccgctacgacctcgactatgaaaaaaactaacagatatcctcgacgccaccatgaaccggacactgaccctgttcaacgtgacccggaatgacgccagagcctacgtgtccggcatccagaatagcgtgtccgccaatagaagcgaccccgtgacactggacgtgctgcctgatagcagctatctgagcggcgccaacctgaacctgagctgccattctgcctctccacagtacagctggcggatcaacggaatccctcagcagcacacccaggtgctgtttatcgccaagatcacccctaacaacaacggcacctacgcctgcttcgtgtccaatctggccaccggcagaaacaacagcatcgtgaagtccatcaccgtgtctgccagcggcacatctcctggactgagcgccacagagtacaagctggtggttgtgggagctgacggcgtgggaaaaagcgccctgacaatccagctgatccagaccgagtataaactcgtggtcgtcggcgctgttggcgttggcaaatctgctctgaccattcagctcattcaggtggccgctgccagacctgttcttcctgctcctgccatcagcgactgggaaagagctagaagaaggcacgtcggccggaaagaaggcgccgctagagctagagttagcctgctgccagagtttggcaccgccgaagttcatctgcctgctcctagcgctgtcagagccgccagaccaggatttggcggagataagggcagactcgtgggcgttgcagaaagacaggtgccctgcagattcctgaggccaacaggcgtgctgagcctgtttcttggcggagctgtggtgtccctgcagtacgtcagacctagcgctctgagaaccctgctggatcagagcgccaaggattgatcaactagccagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 50 - protein, 144 aa, synthetic construct
NRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQ

SEQ ID NO: 51 - protein, 221 aa, synthetic construct
KRYKNRVASRKSRAKFKQLLQHYREVAAAKSSENDRLRLLLKNRTLTLFNVTRNDARAYVSGIQNSVSANRSDPVTLDVLPDSSYLSGANLNLSCHSASPQYSWRINGIPQQHTQVLFIAKITPNNNGTYACFVSNLATGRNNSIVKSITVSASGTSPGLSATEYKLVVVGADGVGKSALTIQLIQSTVHEILSKLSLEGDHSTPPSAYGSVKPYTNFDAE